(19) United States
(12) Patent Application Publication
(10) Pub. No.: US 2023/0061838 A1
(43) Pub. Date: Mar. 2, 2023

(54) Dimeric Antigen Receptors (DAR) That Bind CD20

(71) Applicant: Sorrento Therapeutics, Inc., San Diego, CA (US)

(72) Inventors: Henry Hongjun Ji, Rancho Santa Fe, CA (US); Wenzhong Guo, San Diego, CA (US); Yanliang Zhang, San Diego, CA (US); Bei Bei Ding, San Diego, CA (US); Gunnar F. Kaufmann, San Diego, CA (US)

(73) Assignee: Sorrento Therapeutics, Inc., San Diego, CA (US)

(21) Appl. No.: 17/820,165

(22) Filed: Aug. 16, 2022

Related U.S. Application Data
(63) Continuation of application No. PCT/US2021/020089, filed on Feb. 26, 2021.
(60) Provisional application No. 62/982,348, filed on Feb. 27, 2020.
(60) Provisional application No. 63/089,869, filed on Oct. 9, 2020.

(51) Int. Cl. C07K 16/28; C07K 14/725; C07K 14/705; A61K 35/17; A61P 35/00; C12N 5/0783; C12N 15/62; C12N 15/90

(52) U.S. Cl. CPC C07K 16/2887; C07K 14/7051; C07K 14/70521; A61K 35/17; A61P 35/00; C12N 5/0636; C12N 15/625; C12N 15/907

(57) ABSTRACT

The present disclosure provides dimeric antigen receptors (DAR) constructs that bind a CD20 target antigen, where the DAR construct comprises a heavy chain binding region on one polypeptide chain and a light chain binding region on a separate polypeptide chain. The two polypeptide chains that make up the dimeric antigen receptors can dimerize to form an antigen binding domain. The dimeric antigen receptors have antibody-like properties as they bind specifically to a target antigen. The dimeric antigen receptors can be used for directed cell therapy.

### CROSS REFERENCE TO RELATED APPLICATIONS

This application is a continuation of International Patent Application No. PCT/US2021/020089, filed Feb. 26, 2021, which claims the benefit of priority to U.S. provisional application No. 62/982,348, filed Feb. 27, 2020 and to U.S. provisional application No. 63/089,869, filed Oct. 9, 2020, the contents of each of which are incorporated herein by reference in their entireties.

### SEQUENCE LISTING

The instant application contains a Sequence Listing that has been submitted electronically in XML format and is hereby incorporated by reference in its entirety. Said XML copy, created on Aug. 9, 2022, is named 2022-08-09_01223-0068-00US_ST26.xml and is 86,045 bytes in size.

### TECHNICAL FIELD

The present disclosure provides transgenic cells expressing dimeric antigen receptors (DARs) that bind specifically to CD20, nucleic acids that encode the dimeric antigen receptors, vectors comprising the nucleic acids, and methods of using the transgenic cells for the treatment of disease.

### BACKGROUND

Chimeric antigen receptors (CARs) have been developed to target antigens associated, in particular, with cancer. The first-generation CAR was engineered to contain a signaling domain (TCRζ) that delivers an activation stimulus (signal 1) only (Geiger et al., J. Immunol. 162(10): 5931-5939, 1999; Haynes et al., J. Immunol. 166(1):182-187, 2001) (Hombach et al. Cancer Res. 61(5):1976-1982, 2001; Hombach et al., J. Immunol. 167(11):6123-6131, 2001; Maher et al., Nat. Biotechnol. 20(1):70-75, 2002). T cells grafted with the first-generation CARs alone exhibited limited anti-tumor efficacy due to suboptimal activation (Beecham et al., J. Immunother. 23(6):631-642, 2000). The second-generation CAR, immunoglobulin-CD28-T cell receptor (IgCD28TCR), incorporated a costimulatory CD28 domain (signal 2) into the first-generation receptor (Gerstmayer et al., J. Immunol. 158(10)4584-4590, 1997; Emtage et al., Clin. Cancer Res. 14(24):8112-8122, 2008; Lo, Ma et al., Clin. Cancer Res. 16(10): 2769-2780, 2010) that resulted in CAR-T cells with a greater anti-tumor capacity (Finney et al., J. Immunol. 161(6):2791-2797,1998; Hombach et al., Cancer Res. 61(5):1976-1982, 2001, Maher et al., Nat. Biotechnol. 20(1):70-75, 2002). Various CAR variants have been developed by replacing the signal domains of TCRζ or CD28 with molecules with similar functions, such as FcRγ, 4-1BB and OX40 (Eshhar et al., Proc. Natl. Acad. Sci. USA 90(2):720-724, 1993). TCR CAR-T cells against various tumor antigens have been developed (Ma et al., Cancer Gene Ther. 11(4):297-306, 2004; Ma et al., Prostate 61(1):12-25, 2004; Lo et al., Clin. Cancer Res. 16(10):2769-2780, 2010; Kong et al., Clin. Cancer Res. 18(21):5949-5960, 2012; Ma et al., Prostate 74(3):286-296, 2014; Katz et al., Clin. Cancer Res. 21(14):3149-3159, 2015; Junghans et al., 2016 The Prostate, 76(14):1257-1270).

Adoptive immunotherapy by infusion of T cells engineered with chimeric antigen receptors (CARs) for redirected tumoricidal activity represents a potentially highly specific modality for the treatment of metastatic cancer. CAR-T cells targeting CD19, a molecule expressed on B cells, have shown success in treatment of B cell malignancies and have received FDA approval, with some trials showing a response rate of up to 70%, including sustained complete responses. KYMRIAH® (tisagenlecleucel) CD19 CAR-T cells approved for B cell ALL; YESCARTA® (axicabtagene ciloleucel) CD19 CAR-T cells approved for large B cell lymphoma, and TECARTUS® (brexucabtagene autoleucel) CD19 CAR-T cells approved for large B cell lymphoma.

### SUMMARY

Engineered antigen receptors comprising two polypeptides, a first polypeptide that includes an antibody heavy chain binding region and a second that includes an antibody light chain binding region, are disclosed herein. The engineered receptors can be expressed by cells used therapeutically to provide improved treatment of patients with cancer.

In some embodiments, the present disclosure provides dimeric antigen receptors (DARs) comprising first and second polypeptide chains that include antibody heavy and light chain variable regions and can associate to form a Fab fragment, where one of the polypeptide chains includes transmembrane and intracellular regions of other receptors or immunoglobulin family molecules, anchoring the Fab fragment to the membrane of the host cell and providing signaling capability. The Fab fragment can be directed to a tumor antigen for example. The present disclosure provides nucleic acid constructs encoding DARs and cells transfected with constructs encoding DARs. Cells expressing DARs, such as DAR-T cells, can be used therapeutically, for example can be administered to a patient having cancer for treatment of cancer.

In some embodiments, T cells expressing DARs can show improved efficacy in eradicating tumors in vivo with respect to T cells expressing a CAR.

In some embodiments, T cells expressing DARs can show improved persistence within a subject being treated for cancer with respect to T cells expressing a CAR.

In particular embodiments disclosed herein, the DAR can include a first polypeptide that includes a heavy chain variable region of an antibody that binds CD20 and a second polypeptide that includes a light chain variable region of an antibody that binds CD20. In various embodiments the first polypeptide includes a heavy chain variable region, a heavy chain constant region, a hinge region, a transmembrane region, and at least one intracellular signaling domain. The second polypeptide can include a light chain variable region of the antibody that binds CD20 and can further include a light chain constant region.

In various embodiments, antibody regions can be derived from human antibody sequences, sequences of humanized antibodies, or fully human antibodies.

In various embodiments the first polypeptide includes a heavy chain variable region having the amino acid sequence of SEQ ID NO:3 or having at least 95%, 96%, 97%, 98%, or 99% identity to SEQ ID NO:3. The first polypeptide that includes a heavy chain variable region of an antibody that binds CD20 can further include a heavy chain constant region (e.g., SEQ ID NO:4 or a sequence having at least 95%, 96%, 97%, 98%, or 99% identity thereto), a hinge region (e.g., a CD8 hinge region, a CD28 hinge region, a sequence having at least 95%, 96%, 97%, 98%, or 99% identity to either, or a combination thereof), a transmembrane region (e.g., a CD8 transmembrane region, a 4-1BB transmembrane region, a CD3ζ transmembrane region, a sequence having at least 95%, 96%, 97%, 98%, or 99% identity to any thereof, or a combination of any thereof), and at least one intracellular signaling domain (e.g., a 4-1BB intracellular signaling region, a CD3ζ intracellular signaling, or a combination thereof).

In exemplary embodiments a first polypeptide of an anti-CD20 DAR can include an anti-CD20 antibody heavy chain variable region sequence provided as SEQ ID NO:3 or having at least 95%, 96%, 97%, 98%, or 99% identity to SEQ ID NO:3, the anti-CD20 antibody heavy chain constant region (CH1, SEQ ID NO:4), or an amino acid sequence having at least 95%, 96%, 97%, 98%, or 99% identity to SEQ ID NO:4, a CD28 hinge region (SEQ ID NO:5) or an amino acid sequence having at least 95%, 96%, 97%, 98%, or 99% identity to SEQ ID NO:5, the CD28 transmembrane region (SEQ ID NO:6) or an amino acid sequence having at least 95%, 96%, 97%, 98%, or 99% identity to SEQ ID NO:6, the 4-1BB intracellular costimulatory sequence (SEQ ID NO:7) or an amino acid sequence having at least 95%, 96%, 97%, 98%, or 99% identity to SEQ ID NO:7, and the CD3ζ intracellular signaling region (ITAM3 only) (SEQ ID NO:8) or an amino acid sequence having at least 95%, 96%, 97%, 98%, or 99% identity to SEQ ID NO:8. For example, the first polypeptide of an anti-CD20 DAR can have the sequence of SEQ ID NO:14 or can have at least 95%, 96%, 97%, 98%, or 99% identity to SEQ ID NO:14.

The second polypeptide that includes a light chain variable region of the antibody that binds CD20 (e.g., SEQ ID NO:11 or a sequence having at least 95%, 96%, 97%, 98%, or 99% identity thereto) and can further include a light chain constant region (e.g., SEQ ID NO:12 or a sequence having at least 95%, 96%, 97%, 98%, or 99% identity thereto).

Also included in a further aspect is at least one nucleic acid molecule encoding the first polypeptide of a DAR as provided herein and a second polypeptide of a DAR as provided herein. The encoded polypeptides can include N-terminal signal peptides directing localization of the first and second polypeptides to the cell membrane. Exemplary signal sequences include SEQ ID NO:2 and SEQ ID NO:10. The first and second polypeptides may be encoded on separate nucleic acid molecules or on a single nucleic acid molecule transfected into the intended host cells (e.g., T cells). The first and second polypeptides may be encoded by two open reading frames that, for example, may be operably linked to separate promoters, or may be encoded by a single open reading frame designed for production of two separate proteins by means of a 2A sequence. Alternatively, a nucleic acid sequence encoding the first polypeptide and a nucleic acid sequence encoding the second polypeptide may be operably linked to the same promoter and linked by an IRES sequence for translation from a single transcript. In an exemplary embodiment, a CD20 DAR is encoded by a nucleic acid molecule that encodes the precursor polypeptide of SEQ ID NO:13, or a polypeptide having at least 95%, 96%, 97%, 98%, or 99% identity thereto.

Also included are cells that include a nucleic acid construct encoding an anti-CD20 DAR as provided herein. The cells can express a CD20 DAR. The cells can be T cells, such as primary T cells (DAR-T cells) and can be human primary T cells. In various embodiments the cells produce cytokines in response to co-culturing with tumor cells that express CD20 and/or exhibit cytotoxicity toward tumor cells that express CD20.

In various embodiments cell populations are provided in which the cells have been transfected or transduced with a nucleic acid construct that encodes an anti-CD20 DAR, where at least 20%, at least 30%, at least 40%, at least 50%, at least 60%, at least 70%, at least 80%, or at least 90% of the cells express the CD20 DAR as assessed by flow cytometry. In some embodiments, at least a portion of the cells of the population do not express the T cell receptor, for example, are knocked out in the TRAC gene. The population of T cells having at least 20% of the cells expressing the CD20 DAR and may be depleted of CD3 (T cell receptor) positive cells, e.g., less than 5%, less than 2%, less than 1%, or less than 0.5% of the cell population can express the T cell receptor.

In some embodiments the cells are T cells. In some embodiments the cells are primary T cells (DAR-T cells).

In a further aspect provided herein are methods of treating cancer by administering anti-CD20 DAR-T cells to a subject having cancer. The cells can be administered in a single dose or multiple doses, for example of from about 10⁵ to about 10⁹ cells. The cells can be cells of a population where at least 20% or at least 30% of the cells express the DAR construct and less than 1% of the cells express the endogenous T cell receptor (e.g., less than 1% of the cells are CD3-positive).

Further embodiments according to this disclosure are set forth in the claims and the detailed description.

### DESCRIPTION OF THE DRAWINGS

FIG. 1A is a schematic showing an exemplary dimeric antigen receptor comprising two intracellular signaling sequences.

FIG. 1B is a schematic showing an exemplary dimeric antigen receptor comprising three intracellular signaling sequences.

FIG. 2A is a schematic showing an exemplary dimeric antigen receptor comprising two intracellular signaling sequences.

FIG. 2B is a schematic showing an exemplary dimeric antigen receptor comprising three intracellular signaling sequences.

FIG. 3A is a schematic showing an exemplary precursor polypeptide molecule comprising a self-cleaving sequence and three intracellular signaling sequences.

FIG. 3B is a schematic showing an exemplary precursor polypeptide molecule comprising a self-cleaving sequence and two intracellular signaling sequences.

FIG. 4A is a schematic showing an exemplary precursor polypeptide molecule comprising a self-cleaving sequence and three intracellular signaling sequences.

FIG. 4B is a schematic showing an exemplary precursor polypeptide molecule comprising a self-cleaving sequence and two intracellular signaling sequences.

FIG. 5 is a table listing the naming designation of various embodiments of the anti-CD20 DAR constructs, with their respective hinge and intracellular signaling and costimulatory regions.

FIG. 6A provides flow cytometry results of Cas9-engineered T cells having a knocked-out TRAC gene and either no introduced CAR or DAR construct (leftmost graph), a construct encoding the CD20 CAR of SEQ ID NO:18 (middle graph), or a construct encoding the CD20 DAR of SEQ ID NO:13 (rightmost graph). The y axis provides CD3 expression and the X axis provides expression of CD20 binding constructs. 6B provides flow cytometry results of Cas12a-engineered T cells having a knocked-out TRAC gene and either no introduced CAR or DAR construct (left graph), or the CD20 DAR of SEQ ID NO:13. Axes are as in 6A. Cells analyzed in both A and B were depleted of CD3-positive cells prior to analysis.

FIG. 7 provides graphs showing the % cytotoxicity using Daudi (upper graph) or K562 (lower graph) cells as targets of T cells expressing the CD20 CAR of SEQ ID NO:18 (CAR) and T cells expressing the CD20 DAR of SEQ ID NO:13 (DAR,9:engineered with Cas9; DAR,12: engineered with Cas12a). Effector:Target ratios for assays using Daudi cell targets were 0.06:1, 0.19:1, 0.6:1, 1.7:1, and 5:1. Effector:Target ratios for assays using K562 cell targets were 0.19:1, 0.6:1, 1.7:1, and 5:1.

FIG. 8 provides graphs showing the amount of interferon gamma (IFNγ, upper graph) and granulocyte macrophage colony stimulating factor (GM-CSF, lower graph) secreted by T cells expressing the anti-CD20 CAR of SEQ ID NO:18 and T cells expressing the anti-CD20 DAR of SEQ ID NO:13 after co-culturing with K562 cells, co-culturing with Daudi cells, or culturing alone. From left to right, the bars show results for co-culture with K562 cells, co-culture with Daudi cells, and no additional cells (T cells only) for TRAC KO cells, CD20 DAR-T cells engineered with Cas12a, CD20 DAR-T cells engineered with Cas9, and CD20 CAR-T cells. Black bars are the results from co-culturing with Daudi cells.

FIG. 9 provides graphs showing the results of expanding CD20 CAR and DAR cells by co-culture with CD20-positive cells, with the y axis providing the number of CAR or DAR positive cells. From left to right, the bars show results for co-culture with K562 cells, co-culture with Daudi cells, and no additional cells (IL2 on medium), and no additional cells, with no IL@ I the culture medium for TRAC KO cells, CD20 DAR-T cells engineered with Cas9, and CAR-T cells. Black bars are the results from co-culturing with Daudi cells.

FIG. 10 provides the in vivo images up to 11 weeks after treatment of mice inoculated with Daudi-Fluc tumor cells and then treated with PBS only, TRAC knockout T cells, CD20 CAR-T cells, CD20 DAR-T cells made with Cas9, and CD20 DAR-T cells made with Cas12a (Cpf1).

FIG. 11 is a graph of the total flux average over time of treatment of the tumors of mice shown in FIG. 10. The two curves of mice treated with CD20 DAR-T cells (Cas9-generated and Cas12a-generated) coincide and show no increase over the course of the study.

FIG. 12 is a graph providing body weights of the mice of FIG. 10 over the course of the experiment.

FIG. 13 provides survivorship curves of the mice shown in FIG. 10.

FIG. 14 provides in the left graph the number of human CD45-positive cells detected in peripheral blood of mice treated TCR-KO cells, anti-CD20 CAR-T cells, and anti-CD20 DAR-T cells produced with either Cas9 or Cas12a (Cpf1) as shown in FIG. 10. The right graph provides the number of cells expressing CD20 binding constructs in peripheral blood of mice treated with TCR-KO cells, anti-CD20 CAR-T cells, and anti-CD20 DAR-T cells produced with either Cas9 or Cas12a (Cpf1). Y axes are log scale.

FIG. 15 is a graph providing the percentage of human CD45+ cells in peripheral blood of mice treated with CD20 DAR-T cells, CD20 CAR-T cells, TRAC knockout (TCR KO) T cells, and PBS after inoculation with tumor and following re-challenge with a second tumor inoculum.

FIG. 16 provides the in vivo images up to week 4 of a rechallenge study of CD20 CAR and DAR treated mice. Mice previously inoculated with Daudi-Fluc tumor cells and treated with CD20 CAR-T cells (C), CD20 DAR-T cells made with Cas9 (9), or CD20 DAR-T cells made with Cas12a (12) received a further inoculation with PBS (control) or 5×10⁵, 1×10⁶, 3×10⁶, or 1×10⁷ Daudi-Fluc tumor cells. Each re-inoculation group included 2 mice previously treated with Cas9-engineered DAR-T cells (9), 2 mice previously treated with Cas12a-engineered DAR-T cells (12), and 1 mouse previously treated with CAR-T cells.

FIG. 17A provides flow cytometry analysis of cells eleven days after transfection with a CD20 CAR construct or a CD20 DAR construct, or with the Cas9 RNP in the absence of a CAR construct (TRAC KO). FIG. 17B provides flow cytometry analysis of the CAR-T and DAR-T cells in A after CD3+ cell depletion.

FIG. 18 provides graphs showing the killing of Daudi (left graph) or K562 (right graph) cells by T cells expressing a CD20 CAR (CAR) and T cells expressing a CD20 DAR (DAR) as percent cytotoxicity. Effector:Target ratios for assays using Daudi cell targets were 0.15:1, 0.31:1, 0.6:1, 1.25:1, 2.5:1, and 5:1. Effector:Target ratios for assays using K562 cell targets were 0.6:1, 1.25:1, 2.5:1, and 5:1.

FIG. 19 provides graphs showing the amount of interferon gamma (IFNγ, left graph) and granulocyte macrophage colony stimulating factor (GM-CSF, right graph) secreted by T cells expressing an anti-CD20 CAR and T cells expressing an anti-CD20 DAR after co-culturing with K562 cells, co-culturing with Daudi cells, or culturing alone. From left to right, the bars show results for co-culture with K562 cells, co-culture with Daudi cells, and no co-culture (T cells only) for TRAC KO cells, CD20 CAR-T, and CD20 DAR-T cells. Black bars are the results from co-culturing with Daudi cells.

FIG. 20 provides graphs showing the results of expanding CD20 CAR and CD20 DAR cells by co-culture with CD20-positive cells, with the y axis providing the number of CAR or DAR positive cells. From left to right, the bars show results for co-culture with K562 cells, co-culture with Daudi cells, and no additional cells (IL2 in medium), and no additional cells, with no IL2 in the culture medium for CD20 CAR-T cells and CD20 DAR-T cells. Black bars are the results from co-culturing with Daudi cells.

FIG. 21 provides the in vivo images up to 9 weeks after treatment of mice inoculated with Daudi-Fluc tumor cells and then treated with TRAC knockout T cells, CD20 CAR-T cells, and CD20 DAR-T cells at doses of 6×10⁶ cells, 1.2×10⁶ cells, and 2.4×10⁵ cells.

FIG. 22 is a graph of the total flux average over time of treatment of the tumors of mice shown in FIG. 21. The curves of mice treated with 6×10⁶ CD20 DAR-T cells and 1.2×10⁶ CD20 DAR-T cells coincide and show substantially no increase over the course of the study.

FIG. 23 is a graph providing body weights of the mice of FIG. 21 over the course of the study.

FIG. 24 provides survivorship curves of the mice shown in FIG. 21.

FIG. 25 provides in the first graph the number of human CD45-positive cells detected in peripheral blood of mice treated with TRAC knockout T cells, CD20 CAR-T cells, and CD20 DAR-T cells at doses of 6×10⁶ cells, 1.2×10⁶ cells, and 2.4×10⁵ cells. The second graph provides numbers of DAR-T and CAR-T cells in mice treated with TRAC knockout T cells, CD20 CAR-T cells, and CD20 DAR-T cells at doses of 6×10⁶ cells, 1.2×10⁶ cells, and 2.4×10⁵ cells as measured by peripheral blood cells expressing a CD20 binding construct. Y axes are log scale.

### DETAILED DESCRIPTION

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, N Y, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256:495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

The headings provided herein are not limitations of the various aspects of the disclosure, which aspects can be understood by reference to the specification as a whole.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

The terms “peptide”, “polypeptide”, “polypeptide chain” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules and mature molecule. Precursor molecules include those that have not yet been subjected to post-translation modification such as proteolytic cleavage (including cleavage of a signal peptide directing secretion or membrane insertion of a polypeptide), cleavage due to ribosomal skipping (e.g., mediated by a self-cleaving cleaving sequence such as for example T2A, P2A, E2A or F2A; Donelly et al. (2001) J. Gen. Virol. 82:1013-25; Sharma et al. (2012) Nucl. Acids Res. 40:3143-51), hydroxylation, methylation, lipidation, acetylation, SUMOylation, ubiquitination, glycosylation, fucosylation, phosphorylation, disulfide bond formation, processing of a secretory signal peptide or non-enzymatic cleavage at certain amino acid residues. Polypeptides include mature molecules that have undergone any one or any combination of the post-translation modifications described above. These terms encompass native proteins, recombinant proteins and artificial proteins, protein fragments and polypeptide analogs (such as muteins, variants, chimeric proteins and fusion proteins) of a protein sequence as well as post-translationally, or otherwise covalently or non-covalently, modified proteins. Two or more polypeptides (e.g., 2-6 or more polypeptide chains) can associate with each other, via covalent and/or non-covalent association, to form a polypeptide complex. Association of the polypeptide chains can also include peptide folding. Thus, a polypeptide complex can be dimeric, trimeric, tetrameric, or higher order complexes depending on the number of polypeptide chains that form the complex. Dimeric antigen receptors (DAR) comprising two polypeptide chains are described herein.

The terms “nucleic acid”, “polynucleotide” and “oligonucleotide” and other related terms used herein are used interchangeably and refer to polymers of nucleotides and are not limited to any particular length. The length of a nucleic acid may be referred to in base pairs or nucleotides, which may be used interchangeably regardless of whether a nucleic acid is single-stranded or double-stranded. Nucleic acids include recombinant and chemically-synthesized forms. Nucleic acids include DNA molecules (cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. Nucleic acid molecule can be single-stranded or double-stranded. In one embodiment, the nucleic acid molecules of the disclosure comprise a contiguous open reading frame encoding at least one DAR polypeptide, or a fragment, derivative, mutein, or variant thereof. In one embodiment, nucleic acids comprise one type of polynucleotide or a mixture of two or more different types of polynucleotides. Nucleic acids encoding dimeric antigen receptors (DAR) or antigen-binding portions thereof, are described herein. With respect to embodiments involving a first nucleic acid (e.g., encoding a first polypeptide) and a second nucleic acid (e.g., encoding a second polypeptide), the first nucleic acid and second nucleic acid may be provided either as separate molecules or within the same continuous molecule (e.g., a plasmid or other construct containing first and second coding sequences).

The term “recover” or “recovery” or “recovering”, and other related terms, refers to obtaining a protein (e.g., a DAR or a precursor or antigen binding portion thereof), from host cell culture medium or from host cell lysate or from the host cell membrane. In one embodiment, the protein is expressed by the host cell as a recombinant protein fused to a secretion signal peptide (leader peptide sequence) sequence which mediates secretion of the expressed protein from a host cell (e.g., from a mammalian host cell). The secreted protein can be recovered from the host cell medium. In one embodiment, the protein is expressed by the host cell as a recombinant protein that lacks a secretion signal peptide sequence which can be recovered from the host cell lysate. In one embodiment, the protein is expressed by the host cell as a membrane-bound protein which can be recovered using a detergent to release the expressed protein from the host cell membrane. In one embodiment, irrespective of the method used to recover the protein, the protein can be subjected to procedures that remove cellular debris from the recovered protein. For example, the recovered protein can be subjected to chromatography, gel electrophoresis and/or dialysis. In one embodiment, the chromatography comprises any one or any combination or two or more procedures including affinity chromatography, hydroxyapatite chromatography, ion-exchange chromatography, reverse phase chromatography and/or chromatography on silica. In one embodiment, affinity chromatography comprises protein A or G (cell wall components from Staphylococcus aureus).

The term “isolated” refers to a protein (e.g., A DAR or a precursor or antigen binding portion thereof) or polynucleotide that is substantially free of other cellular material. A protein may be rendered substantially free of naturally associated components (or components associated with a cellular expression system or chemical synthesis methods used to produce the DAR) by isolation, using protein purification techniques well known in the art. The term isolated also refers in some embodiment to protein or polynucleotides that are substantially free of other molecules of the same species, for example other protein or polynucleotides having different amino acid or nucleotide sequences, respectively. The purity of homogeneity of the desired molecule can be assayed using techniques well known in the art, including low resolution methods such as gel electrophoresis and high resolution methods such as HPLC or mass spectrometry. In one embodiment, isolated precursor polypeptides, and first and second polypeptide chains, of the dimeric antigen receptor (DAR) or antigen-binding portions thereof, of the present disclosure are isolated.

The term “precursor polypeptide(s)” or related terms, may be used herein to refer to a precursor polypeptide that can be processed to become first and second polypeptide chains that associate/assemble to form dimeric antigen receptors (DAR) constructs. In any of the precursor polypeptide embodiments described herein that comprise a self-cleaving sequence, the self-cleaving sequence may be a T2A, P2A, E2A, or F2A sequence. The precursor polypeptide can be processed by cleaving at the self-cleaving sequence to release the first and second polypeptide chains and secreting a polypeptide chain, for example the second polypeptide chain of a DAR, and/or anchoring a polypeptide chain, for example the first polypeptide chain of a DAR, in a cellular membrane. A precursor polypeptide can include one or more signal peptides that can be cleaved on insertion of a first polypeptide into the cell membrane and/or secretion of a second polypeptide from the cell. The first and second polypeptide chains can dimerize via at least one disulfide bond between the antibody heavy chain constant region and the antibody light chain constant region, and the antibody heavy chain variable region and the antibody light chain variable region can form an antigen binding domain that binds a CD20 antigen.

The term “leader sequence” or “leader peptide” or “peptide signal sequence” or “signal peptide” or “secretion signal peptide” refers to a peptide sequence that is located at the N-terminus of a polypeptide. A leader sequence directs a polypeptide chain to a cellular secretory pathway and can direct integration and anchoring of the polypeptide into the lipid bilayer of the cellular membrane. Typically, a leader sequence is about 10-50 amino acids in length. A leader sequence can direct transport of a precursor polypeptide from the cytosol to the endoplasmic reticulum. In one embodiment, a leader sequence includes signal sequences comprising CD8α, CD28, or CD16 leader sequences. In one embodiment, the signal sequence comprises a mammalian sequence, including for example mouse or human Ig gamma secretion signal peptide. In some embodiments, a leader sequence comprises a mouse Ig gamma leader peptide sequence MEWSWVFLFFLSVTTGVHS (SEQ ID NO:2) or MSVPTQVLGLLLLWLTDARC (SEQ ID NO:10).

An “antigen binding protein” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include dimeric antigen receptors (DARs), antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold. Antigen binding proteins comprising dimeric antigen receptors (DAR) are described herein.

An antigen binding protein can have, for example, the structure of an immunoglobulin. In one embodiment, an “immunoglobulin” refers to a tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The heavy and/or light chains may or may not include a leader sequence for secretion. The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules. Antigen binding proteins having dimeric antigen receptor (DAR) structures with immunoglobulin-like properties that bind specifically to a target antigen (e.g., CD20 antigen) are described herein.

The variable regions of immunoglobulin chains exhibit the same general structure of relatively conserved framework regions (FR) joined by three hypervariable regions, also called complementarity determining regions or CDRs. From N-terminus to C-terminus, both light and heavy chains comprise the segments FR1, CDR1, FR2, CDR2, FR3, CDR3 and FR4.

One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991 (“Kabat numbering”). Other numbering systems for the amino acids in immunoglobulin chains include IMGT® (international ImMunoGeneTics information system; Lefranc et al, Dev. Comp. Immunol. 29:185-203; 2005) and AHo (Honegger and Pluckthun, J. Mol. Biol. 309(3):657-670; 2001); Chothia (Al-Lazikani et al., 1997 Journal of Molecular Biology 273:927-948; Contact (Maccallum et al., 1996 Journal of Molecular Biology 262:732-745, and Aho (Honegger and Pluckthun 2001 Journal of Molecular Biology 309:657-670.

An “antibody” and “antibodies” and related terms used herein refers to an intact immunoglobulin or to an antigen binding portion thereof that binds specifically to an antigen. Antigen binding portions may be produced by recombinant DNA techniques or by enzymatic or chemical cleavage of intact antibodies. Antigen binding portions include, inter alia, Fab, Fab′, F(ab′)2, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)2 fragments, Fab′ fragments and Fab fragments. Antibodies include single domain antibodies (nanobodies), monovalent antibodies, single chain antibodies, single chain variable fragment (scFv), camelized antibodies, affibodies, disulfide-linked Fvs (sdFv), anti-idiotypic antibodies (anti-Id), and minibodies. Antibodies include monoclonal and polyclonal populations. Dimeric antigen receptors (DAR) comprising antibodies are described herein.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains. Dimeric antigen receptors (DAR) having antibody heavy chain variable regions and antibody light chain variable regions that form antigen binding domains are described herein.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). In one embodiment, an antibody specifically binds to a target antigen if it binds to the antigen with a dissociation constant K_{D} of 10⁻⁵ M or less, or 10⁻⁶ M or less, or 10⁻⁷ M or less, or 10⁻⁸ M or less, or 10⁻⁹ M or less, or 10⁻¹⁰ M or less, or 10⁻¹¹ M or less. In one embodiment, dimeric antigen receptors (DAR) that bind specifically to their target antigen (e.g., CD20 antigen) are described herein.

In one embodiment, binding specificity of an antibody or antigen binding protein or antibody fragment can be measure by ELISA, radioimmune assay (RIA), electrochemiluminescence assays (ECL), immunoradiometric assay (IRMA), or enzyme immune assay (EIA).

In one embodiment, a dissociation constant (K_{D}) can be measured using a BIACORE surface plasmon resonance (SPR) assay. Surface plasmon resonance refers to an optical phenomenon that allows for the analysis of real-time interactions by detection of alterations in protein concentrations within a biosensor matrix, for example using the BIACORE system (Biacore Life Sciences division of GE Healthcare, Piscataway, N.J.).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope. In one embodiment, dimeric antigen receptors (DAR) or antigen-binding portions thereof that bind an epitope of CD20 antigen are described herein.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)2, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment. In one embodiment, dimeric antigen receptors comprising a Fab fragment joined to a hinge, transmembrane and intracellular regions are described herein.

The terms “Fab”, “Fab fragment” and other related terms refers to a monovalent fragment comprising a variable light chain region (V_{L}), constant light chain region (C_{L}), variable heavy chain region (V_{H}), and first constant region (C_{H1}). A Fab is capable of binding an antigen. An F(ab′)₂ fragment is a bivalent fragment comprising two Fab fragments linked by a disulfide bridge at the hinge region. A F(Ab′)₂ has antigen binding capability. An Fd fragment comprises V_{H} and C_{H1} regions. An Fv fragment comprises V_{L} and V_{H} regions. An Fv can bind an antigen. A dAb fragment has a V_{H} domain, a V_{L} domain, or an antigen-binding fragment of a V_{H} or V_{L} domain (U.S. Pat. Nos. 6,846,634 and 6,696,245; U.S. published Application Nos. 2002/02512, 2004/0202995, 2004/0038291, 2004/0009507, 2003/0039958; and Ward et al., Nature 341:544-546, 1989). In one embodiment, dimeric antigen receptors comprising a Fab fragment joined to a hinge, transmembrane and intracellular regions are described herein.

The term “human antibody” refers to antibodies that have one or more variable and constant regions derived from human immunoglobulin sequences. In one embodiment, all of the variable and constant domains are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes. Dimeric antigen receptors (DAR) comprising fully human antibody heavy chain variable region and fully human antibody light chain variable regions are described herein.

A “humanized” antibody refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293. In some embodiments of a DAR or precursor thereof described herein, the heavy chain variable domain and light chain variable domain of the DAR or precursor thereof are humanized.

The term “chimeric antibody” and related terms used herein refers to an antibody that contains one or more regions from a first antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen). Chimeric antibodies can be prepared from portions of any of the dimeric antigen receptor (DAR) antigen-binding portions thereof are described herein.

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides.

As used herein, the term “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising full-length heavy chains and full-length light chains, derivatives, variants, fragments, and muteins thereof, examples of which are described below.

The term “hinge” refers to an amino acid segment that is generally found between two domains of a protein and may allow for flexibility of the overall construct and movement of one or both of the domains relative to one another. Structurally, a hinge region comprises from about 10 to about 100 amino acids, e.g., from about 15 to about 75 amino acids, from about 20 to about 50 amino acids, or from about 30 to about 60 amino acids. In some embodiments, a hinge region in a polypeptide such as a DAR polypeptide is 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 amino acids in length. A hinge region can be derived from is a hinge region of a naturally-occurring protein, such as a CD8 hinge region or a fragment thereof, a CD8α hinge region, or a fragment thereof, a hinge region of an antibody (e.g., IgG, IgA, IgM, IgE, or IgD antibodies), or a hinge region that joins the constant domains CH1 and CH2 of an antibody. The hinge region can be derived from an antibody and may or may not comprise one or more constant regions of the antibody, or the hinge region comprises the hinge region of an antibody and the CH3 constant region of the antibody, or the hinge region comprises the hinge region of an antibody and the CH2 and CH3 constant regions of the antibody, or the hinge region is a non-naturally occurring peptide, or the hinge region is disposed between the C-terminus of the scFv and the N-terminus of the transmembrane domain. In some embodiments, the hinge region comprises any one or any combination of two or more regions comprising an upper, core or lower hinge sequences from an IgG1, IgG2, IgG3 or IgG4 immunoglobulin molecule. In some embodiments of DARs, a hinge region comprises an IgG1 upper hinge sequence EPKSCDKTHT (SEQ ID NO: 59). In some DAR embodiments, a hinge region comprises an IgG1 core hinge sequence CPX̅C, wherein X̅ is P, R or S (SEQ ID NO: 60). In some embodiments, a hinge region comprises a lower hinge/CH2 sequence PAPELLGGP (SEQ ID NO: 56). In some embodiments, the hinge is joined to an Fc region (CH2) having the amino acid sequence SVFLFPPKPKDT (SEQ ID NO: 57). In some embodiments, the hinge region includes the amino acid sequence of an upper, core and lower hinge and comprises EPKSCDKTHTCPPCPAP ELLGGP (SEQ ID NO: 58). In some embodiments, the hinge region comprises one, two, three or more cysteines that can form at least one, two, three or more interchain disulfide bonds.

The term “Fc” or “Fc region” as used herein refers to the portion of an antibody heavy chain constant region beginning in or after the hinge region and ending at the C-terminus of the heavy chain. The Fc region comprises at least a portion of the CH2 and CH3 regions, and may or may not include a portion of the hinge region. An Fc region can bind Fc cell surface receptors and some proteins of the immune complement system. An Fc region exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. In one embodiment, the Fc region can include a mutation that increases or decreases any one or any combination of these functions. In one embodiment, the Fc domain comprises a LALA-PG mutation (L234A, L235A, P329G) which reduces effector function. In one embodiment, the Fc domain mediates serum half-life of the protein complex, and a mutation in the Fc domain can increase or decrease the serum half-life of the protein complex. In one embodiment, the Fc domain affects thermal stability of the protein complex, and mutation in the Fc domain can increase or decrease the thermal stability of the protein complex. An Fc region can bind an Fc receptor, including FcγRI (e.g., CD64), FcγRII (e.g., CD32) and/or FcγRIII (e.g., CD16a). An Fc region can bind a complement component C1q.

The term “labeled” or related terms as used herein with respect to a polypeptide refers to joinder thereof to a detectable label or moiety for detection. Exemplary detectable labels or moieties include radioactive, colorimetric, antigenic, or enzymatic labels/moieties, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens). Any of the dimeric antigen receptors (DAR) or antigen-binding portions thereof that described herein can be unlabeled or can be joined to a detectable label or detectable moiety.

The “percent identity” or “percent homology” and related terms used herein refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters. Expressions such as “comprises a sequence with at least X % identity to Y” with respect to a test sequence mean that, when aligned to sequence Y as described above, the test sequence comprises residues identical to at least X % of the residues of Y.

In one embodiment, the amino acid sequence of a test construct (e.g., DAR) may be similar but not necessarily identical to any of the amino acid sequences of the polypeptides that make up a given dimeric antigen receptor (DAR) or antigen-binding portions thereof that are described herein. The similarities between the test construct and the polypeptides can be at least 95%, or at or at least 96% identical, or at least 97% identical, or at least 98% identical, or at least 99% identical, to any of the polypeptides that make up the DAR or antigen-binding portions thereof that are described herein. In one embodiment, similar polypeptides can contain amino acid substitutions within a heavy and/or light chain. In one embodiment, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Further, one of skill in the art can consider, based on knowledge and comparison of conserved protein domains, for example, among proteins with similar function as well as, for example, known crystal structures and modeling, which regions of a protein are unlikely to result in disrupting a desired function of the protein and may therefore be modified without deleterious affects.

Antibodies, including the dimeric antigen receptors (DAR) described herein can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecific antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen. Antibodies can be produced using recombinant nucleic acid technology as described below.

The term “Chimeric Antigen Receptor” or “CAR” refers to a single chain fusion protein comprising an extracellular antigen-binding protein that is fused to an intracellular domain. The CAR extracellular binding domain is a single chain variable fragment (scFv or sFv) derived from fusing the variable heavy and light regions of a monoclonal antibody, such as a human monoclonal antibody. In one embodiment, a CAR comprises (i) an antigen binding protein comprising a heavy chain variable (VH) domain and a light chain variable (VL) domain wherein the VH and VL domains are joined together by a peptide linker; (ii) a hinge domain, (iii) a transmembrane domain; and (iv) an intracellular domain comprising an intracellular signaling sequence. Disclosed herein are Dimeric Antigen Receptors (DARs) which are distinct from CARs in that DARs do not use a single chain antibody for targeting but instead use heavy and light chain variable domain regions on separate polypeptide chains that associate with one another to form an Fab-like binding domain on the host cell surface.

A “vector” and related terms used herein refers to a nucleic acid molecule (e.g., DNA or RNA) which can be operably linked to foreign genetic material (e.g., nucleic acid transgene) and include at least one of: one or more promoters, one or more recombination sequences, one or more origins of replication or autonomous replication sequences, and one or more selectable or detectable markers. Vectors can be used as a vehicle to introduce foreign genetic material into a cell (e.g., host cell). Vectors can include at least one restriction endonuclease recognition sequence for insertion of the transgene into the vector. Vectors can include at least one gene sequence that confers antibiotic resistance or a selectable characteristic to aid in selection of host cells that harbor a vector-transgene construct. Vectors can be single-stranded or double-stranded nucleic acid molecules. Vectors can be linear or circular nucleic acid molecules. One type of vector is a “plasmid,” which refers to a linear or circular double stranded extrachromosomal DNA molecule which can be linked to a transgene, and is capable of replicating in a host cell, and may be configured for transcribing the transgene (for example, includes a promoter and optionally other gene regulatory sequences for expression of an operably linked transgene). A viral vector typically contains viral RNA or DNA backbone sequences which can be linked to the transgene. The viral backbone sequences can be modified to disable infection but retain insertion of the viral backbone and the co-linked transgene into a host cell genome. Examples of viral vectors include retroviral, lentiviral, adenoviral, adeno-associated, baculoviral, papovaviral, vaccinia viral, herpes simplex viral and Epstein Barr viral vectors. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome.

An “expression vector” is a type of vector that can contain one or more regulatory sequences, such as inducible and/or constitutive promoters and enhancers. Expression vectors can include ribosomal binding sites and/or polyadenylation sites. Expression vectors can optionally include one or more origin of replication sequence. Regulatory sequences direct transcription, or transcription and translation, of a transgene linked to the expression vector which is transduced into a host cell. The regulatory sequence(s) can control the level, timing and/or location of expression of the transgene. The regulatory sequence can, for example, exert its effects directly on the transgene, or through the action of one or more other molecules (e.g., polypeptides that bind to the regulatory sequence and/or the nucleic acid). Regulatory sequences can be part of a vector. Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-3606. An expression vector can comprise nucleic acids that encode at least a portion of any of the dimeric antigen receptors (DAR) or antigen-binding portions thereof that are described herein.

A transgene is “operably linked” to a vector when there is linkage between the transgene and the vector to permit functioning or expression of the transgene sequences contained in the vector. In one embodiment, a transgene is “operably linked” to a regulatory sequence when the regulatory sequence affects the expression (e.g., the level, timing, or location of expression) of the transgene.

The terms “transfected” or “transformed” or “transduced” or other related terms used herein refer to a process by which exogenous nucleic acid (e.g., transgene) is transferred or introduced into a host cell. A “transfected” or “transformed” or “transduced” host cell is one into which an exogenous nucleic acid (for example, including a transgene) has been introduced. “Transduced” is typically used to indicate gene transfer by means of a virus (e.g., a retrovirus or lentivirus). The term host cell includes the primary subject cell and its progeny. Exogenous nucleic acids encoding at least a portion of any of the dimeric antigen receptors (DARs) or antigen-binding portions thereof that are described herein can be introduced into a host cell. Expression vectors or DNA fragments comprising at least a portion of any of the dimeric antigen receptors (DAR) or antigen-binding portions thereof that are described herein can be introduced into a host cell, and the host cell can express polypeptides comprising at least a portion of the DAR or antigen-binding portions thereof that are described herein.

In various embodiments, a host cell can be introduced with an expression vector or nucleic acid fragment in which a promoter is operably linked to a nucleic acid sequence encoding a DAR thereby generating a transfected/transformed host cell which is cultured under conditions suitable for expression of the DAR by the transfected/transformed host cell.

Typically, a host cell is a cultured cell that can be transformed or transfected with a polypeptide-encoding nucleic acid, which can then be expressed in the host cell. The phrase “transgenic host cell” or “recombinant host cell” can be used to denote a host cell that has been introduced (e.g., transduced, transformed, or transfected) with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein. A host cell, or a population of host cells, harboring a vector (e.g., an expression vector) operably linked to at least one nucleic acid encoding one or more polypeptides that comprise a dimeric antigen receptor (DAR) or antigen-binding portions thereof are described herein.

A foreign nucleic acid introduced into cells can comprise an expression vector having a promoter operably linked to a transgene, and the host cell can be used to express the nucleic acid and/or polypeptide encoded by the foreign nucleic acid (transgene). A host cell (or a population thereof) can be a cultured cell or can be extracted from a subject. A cultured cell can be a cell of a cell line or a primary cell. The host cell (or a population thereof) includes the primary subject cell and its progeny without any regard for the number of passages. The host cell (or a population thereof) includes immortalized cell lines. Host cells encompass progeny cells. Progeny cells may or may not harbor identical genetic material compared to the parent cell. In one embodiment, a host cell describes any cell (including its progeny) that has been modified, transfected, transduced, transformed, and/or manipulated in any way to express a DAR, as disclosed herein. In one example, the host cell (or population thereof) can be introduced with an expression vector operably linked to a nucleic acid encoding the desired antibody, or an antigen binding portion thereof, described herein. Host cells and populations thereof can harbor an expression vector, including a retroviral or lentiviral vector or portion thereof, that is stably integrated into the host's genome, or can harbor an extrachromosomal expression vector. In one embodiment, host cells and populations thereof can harbor an extrachromosomal vector that is present after several cell divisions or is present transiently and is lost after several cell divisions.

The terms “host cell” or “population of host cells” or related terms as used herein refer to a cell (or a population of cells) into which foreign (exogenous or transgene) nucleic acids have been introduced. The term “population of host cells” can refer to a population of cells, particularly primary cells, that has been transfected or transduced with an exogenous nucleic acid sequence encoding, for example, a DAR, where DAR-expressing cells may represent less than 100% of the population. For example, a population of host cells transfected with a DAR construct may comprise at least 5%, at least 10%, at least 20%, at least 25%, at least 30%, at least 35%, at least 40%, or at least 50% cells that express the DAR. The percentage of cells of the host cell population that expresses a gene of interest, such as a DAR-encoding gene, can optionally be increased, for example, by flow cytometry, selective capture or DAR-positive cells, or by expansion on the DAR binding partner or cells expressing the DAR binding partner (e.g., CD20 where the DAR is a CD20 DAR).

Polypeptides of the present disclosure (e.g., dimeric antigen receptors (DAR)) can be produced using any method known in the art. In one example, the polypeptides are produced by recombinant nucleic acid methods by inserting a nucleic acid sequence (e.g., DNA) encoding the polypeptide into a recombinant expression vector which is introduced into a host cell and expressed by the host cell under conditions promoting expression.

General techniques for recombinant nucleic acid manipulations are described for example in Sambrook et al., in Molecular Cloning: A Laboratory Manual, Vols. 1-3, Cold Spring Harbor Laboratory Press, 2 ed., 1989, or F. Ausubel et al., in Current Protocols in Molecular Biology (Green Publishing and Wiley-Interscience: New York, 1987) and periodic updates, herein incorporated by reference in their entireties. The nucleic acid (e.g., DNA) encoding the polypeptide is operably linked to an expression vector carrying one or more suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The expression vector can include an origin or replication that confers replication capabilities in the host cell. The expression vector can include a gene that confers selection to facilitate recognition of transgenic host cells (e.g., transformants).

The recombinant DNA can also encode any type of protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985).

The expression vector construct can be introduced into the host cell using a method appropriate for the host cell. A variety of methods for introducing nucleic acids into host cells are known in the art, including, but not limited to, electroporation; transfection employing calcium chloride, rubidium chloride, calcium phosphate, DEAE-dextran, or other substances; viral transfection; non-viral transfection; microprojectile bombardment; lipofection; and infection (e.g., where the vector is an infectious agent). Suitable host cells include prokaryotes, yeast, mammalian cells, or bacterial cells.

A host cell can be a prokaryote, for example, E. coli, or it can be a eukaryote, for example, a single-celled eukaryote (e.g., a yeast or other fungus), a plant cell (e.g., a tobacco or tomato plant cell), a mammalian cell (e.g., a human cell, a monkey cell, a hamster cell, a rat cell, a mouse cell, or an insect cell) or a hybridoma. In various embodiments, host cells comprise non-human cells including CHO, BHK, NS0, SP2/0, and YB2/0. In other embodiments, host cells comprise human cells including HEK293, HT-1080, Huh-7 and PER.C6. In some embodiments, a host cell is a mammalian host cell, but is not a human host cell. Examples of host cells include the COS-7 line of monkey kidney cells (ATCC CRL 1651) (see Gluzman et al., 1981, Cell 23: 175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31) or CHO strain DX-B 11, which is deficient in DHFR (see Urlaub et al., 1980, Proc. Natl. Acad. Sci. USA 77:4216-20), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo 205 cells, other transformed primate cell lines, normal diploid cells, cell strains derived from in vitro culture of primary tissue, primary explants, HL-60, U937, HaK or Jurkat cells. In one embodiment, host cells include lymphoid cells such as Y0, NS0 or Sp20.

Suitable bacteria include gram negative or gram positive organisms, for example, E. coli or Bacillus spp. Yeast, for example from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow and Summers, (Bio/Technology, 6:47, 1988). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. The protein is then purified from culture media or cell extracts. Any of the polypeptide chains that comprise the dimeric antigen receptors (DAR) or antigen-binding portions thereof, can be expressed by transgenic host cells.

Antibodies and antigen binding proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

Nucleic acids encoding any of the various polypeptides disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA. 2003 100(2):438-42; Sinclair et al. Protein Expr. Purif. 2002 (1):96-105; Connell N D. Curr. Opin. Biotechnol. 2001 12(5):446-9; Makrides et al. Microbiol. Rev. 1996 60(3):512-38; and Sharp et al. Yeast. 1991 7(7):657-78.

Antibodies and antigen binding proteins described herein can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

Antibodies and antigen binding proteins described herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified antibodies and antigen binding proteins described herein are at least 65% pure, at least 75% pure, at least 85% pure, at least 95% pure, or at least 98% pure. Regardless of the exact numerical value of the purity, the polypeptide is sufficiently pure for use as a pharmaceutical product. Any of the dimeric antigen receptors (DAR) or antigen-binding portions thereof that are described herein can be expressed by transgenic host cells and then purified to about 65-98% purity or high level of purity using any art-known method.

In certain embodiments, the antibodies and antigen binding proteins described herein (e.g., DAR) can further comprise post-translational modifications. Exemplary post-translational protein modifications include phosphorylation, acetylation, methylation, ADP-ribosylation, ubiquitination, glycosylation, afucosylation, carbonylation, sumoylation, biotinylation or addition of a polypeptide side chain or of a hydrophobic group. As a result, the modified polypeptides may contain non-amino acid elements, such as lipids, poly- or mono-saccharide, and phosphates. In one embodiment, glycosylation can be sialylation, which conjugates one or more sialic acid moieties to the polypeptide. Sialic acid moieties improve solubility and serum half-life while also reducing the possible immunogenicity of the protein. See Raju et al. Biochemistry. 2001 31; 40(30):8868-76.

In one embodiment, the dimeric antigen receptors (DAR) described herein can be modified to become soluble polypeptides which comprises linking the antibodies and antigen binding proteins to non-proteinaceous polymers. In one embodiment, the non-proteinaceous polymer comprises polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337.

The present disclosure provides therapeutic compositions comprising any of the dimeric antigen receptors (DAR) that are described herein, or cells or cell populations described herein (e.g., expressing a DAR described herein) in an admixture with a pharmaceutically-acceptable excipient. Excipients encompass, for example, carriers, stabilizers, diluents or fillers (e.g., sucrose and sorbitol), lubricating agents, glidants, and anti-adhesives (e.g., magnesium stearate, zinc stearate, stearic acid, silicas, hydrogenated vegetable oils, or talc). Additional examples include buffering agents, stabilizing agents, preservatives, non-ionic detergents, anti-oxidants and isotonifiers. Where a therapeutic composition comprises cells, the pharmaceutically-acceptable excipients will be chosen so as not to interfere with the viability or activity of the cells.

Therapeutic compositions and methods for preparing them are well known in the art and are found, for example, in “Remington: The Science and Practice of Pharmacy” (20th ed., ed. A. R. Gennaro A R., 2000, Lippincott Williams & Wilkins, Philadelphia, Pa.). Therapeutic compositions can be formulated for parenteral administration may, and can for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the antibody (or antigen binding protein thereof) described herein. Nanoparticulate formulations (e.g., biodegradable nanoparticles, solid lipid nanoparticles, liposomes) may be used to control the biodistribution of the antibody (or antigen binding protein thereof). Other potentially useful parenteral delivery systems include ethylene-vinyl acetate copolymer particles, osmotic pumps, implantable infusion systems, and liposomes. The concentration of the antibody (or antigen binding protein thereof) in the formulation varies depending upon a number of factors, including the dosage of the drug to be administered, and the route of administration.

Any of the dimeric antigen receptors (DAR) or antigen-binding portions thereof described herein may be administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like. In one example, the DAR (or antigen binding portions thereof) is formulated in the presence of sodium acetate to increase thermal stability.

The term “subject” as used herein refers to human and non-human animals, including vertebrates, mammals and non-mammals. In one embodiment, the subject can be human, non-human primates, simian, ape, murine (e.g., mice and rats), bovine, porcine, equine, canine, feline, caprine, lupine, ranine or piscine.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. Cells expressing DARs will generally be delivered by infusion or injection. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods. Any of the dimeric antigen receptors (DAR) or antigen-binding portions thereof described herein can be administered to a subject using art-known methods and delivery routes.

The terms “effective amount”, “therapeutically effective amount” or “effective dose” or related terms may be used interchangeably and for DAR-expressing cells refer the number of cells expressing a DAR, e.g., DAR-T cells, as described herein that when administered to a subject, is sufficient to effect a measurable improvement or prevention of a disease or disorder associated with tumor or cancer antigen expression. Therapeutically effective amounts of DAR-expressing cells as provided herein, when used alone or in combination, will vary depending upon the relative effectiveness of the cells (e.g., in inhibiting cell growth) and depending upon the subject and disease condition being treated, the weight and age and sex of the subject, the severity of the disease condition in the subject, the manner of administration and the like, which can readily be determined by one of ordinary skill in the art.

In one embodiment, a therapeutically effective amount comprises a dose of about 10³-10¹² transgenic host cells administered to the subject. The transgenic host cells can harbor one or more nucleic acids that encode the polypeptide chains that comprise any of the DARs described herein. The therapeutically effective amount can be determined by considering the subject to receive the therapeutically effective amount and the disease/disorder to be treated which may be ascertained by one skilled in the art using known techniques. The therapeutically effective amount may consider factors pertaining to the subject such as age, body weight, general health, sex, diet, time of administration, drug interaction, and the severity of the disease/disorder. The therapeutically effective amount may consider the purity of the transgenic host cells and the percentage of DAR-expressing cells within the population, which can be about 10%-98% or higher levels of purity. The therapeutically effective amount of the transgenic host cells can be administered to the subject at least once, or twice, three times, 4 times, 5 times, or more over a period of time. The period of time can be per day, per week, per month, or per year. The therapeutically effective amount of the transgenic cells administered to the subject can be the same each time or can be increased or decreased at each administration event. In some embodiments, the therapeutically effective amount of the transgenic cells may be administered to the subject until the tumor size or number of cancer cells is reduced by 5%-90% or more, compared to the tumor size or number of cancer cells prior to administration of the transgenic host cells.

The present disclosure provides methods for treating a subject having a disease/disorder associated with expression or over-expression of one or more tumor-associated antigens. The disease comprises cancer or tumor cells expressing the tumor-associated antigens, such as for example CD20 antigen. In various embodiments, the cancer or tumor includes cancer of the prostate, breast, ovary, head and neck, bladder, skin, colorectal, anus, rectum, pancreas, lung (including non-small cell lung and small cell lung cancers), leiomyoma, brain, glioma, glioblastoma, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis and testis.

In further embodiments, the cancer comprises hematological cancers, including leukemias, lymphomas, myelomas and B cell lymphomas. Hematologic cancers include multiple myeloma (MM), non-Hodgkin's lymphoma (NHL) including Burkitt's lymphoma (BL), B chronic lymphocytic leukemia (B-CLL), systemic lupus erythematosus (SLE), B and T acute lymphocytic leukemia (ALL), acute myeloid leukemia (AML), chronic lymphocytic leukemia (CLL), diffuse large B cell lymphoma, chronic myelogenous leukemia (CML), hairy cell leukemia (HCL), follicular lymphoma, Waldenstrom's Macroglobulinemia, mantle cell lymphoma, Hodgkin's Lymphoma (HL), plasma cell myeloma, precursor B cell lymphoblastic leukemia/lymphoma, plasmacytoma, giant cell myeloma, plasma cell myeloma, heavy-chain myeloma, light chain or Bence-Jones myeloma, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas' disease, Grrave's disease, Wegener's granulomatosis, poly-arteritis nodosa, Sjogren's syndrome, pemphigus vulgaris, scleroderma, multiple sclerosis, anti-phospholipid syndrome, ANCA associated vasculitis, Goodpasture's disease, Kawasaki disease, autoimmune hemolytic anemia, and rapidly progressive glomerulonephritis, heavy-chain disease, primary or immunocyte-associated amyloidosis, and monoclonal gammopathy of undetermined significance.

### Dimeric Antigen Receptors (DARs)

The present disclosure provides dimeric antigen receptors (DARs) comprising two polypeptides, where the association of the first and second polypeptide form a Fab fragment joined to a transmembrane domain and intracellular domains. In some embodiments, a DAR includes an optional hinge region between the Fab fragment moiety and the transmembrane region. In some embodiments, the presently disclosed DAR structures provide unexpected and surprising results, e.g., based on comparing a DAR structure having a Fab format antibody to a CAR structure having an scFv format of the same antibody. In various instances, and as illustrated in the Examples provided herein, a DAR and CAR having the same hinge region, transmembrane domain, and intracellular domain(s) can be compared. A DAR format may provide superior results relative to the corresponding CAR format in, for example, binding to cells that express the target antigen, antigen-induced cytokine release, and/or antigen-induced cytotoxicity. In some examples, cells expressing a DAR can exhibit greater selectivity for cells expressing the target to which the DAR is directed in cytotoxicity, cytokine release, and/or clonal expansion than are exhibited by cells expressing a corresponding CAR differing only in the antibody format. In some examples, transgenic cells expressing a DAR demonstrate more efficient in vivo eradication of tumor, greater persistence in a treated subject, and greater effectiveness in preventing tumor re-establishment in a previously treated subject than is observed for transgenic cells expressing a CAR that includes the same hinge, transmembrane, and intracellular regions and the same heavy chain variable and light chain variable regions as the DAR.

The present disclosure provides dimeric antigen receptor (DAR) constructs comprising a heavy chain binding region on one polypeptide chain and a light chain binding region on a separate polypeptide chain. The two polypeptide chains that make up the dimeric antigen receptors can dimerize to form a protein complex. The dimeric antigen receptors have antibody-like properties as they bind specifically to a target antigen. The dimeric antigen receptors can be used for directed cell therapy.

The present disclosure provides transgenic T cells engineered to express anti-CD20 dimeric antigen receptor (DAR) constructs having an antigen-binding extracellular portion and an intracellular co-stimulatory and/or intracellular signaling portion. The extracellular portion exhibits high affinity and avidity to bind CD20-expressing diseased hematopoietic cells leading to T cell activation and diseased-cell killing. The intracellular portion comprises co-stimulatory and/or signaling regions that mediate T cell activation upon antigen binding which can lead to enhanced T cell expansion and/or formation of memory T cells that express the CD20 DAR constructs. It is postulated that formation of memory T cells is important to prevent disease relapse in a subject suffering from a hematologic disease involving CD20-overexpression. Described herein are multiple configurations of DAR constructs that differ in the type and number of intracellular co-stimulatory and signaling regions, providing flexibility in designing DAR constructs for producing a strong and rapid effector response (e.g., DAR constructs comprising an intracellular CD28 co-stimulatory region) and/or generating a longer-lasting memory T cell population (e.g., DAR constructs comprising an intracellular 4-1BB co-stimulatory region).

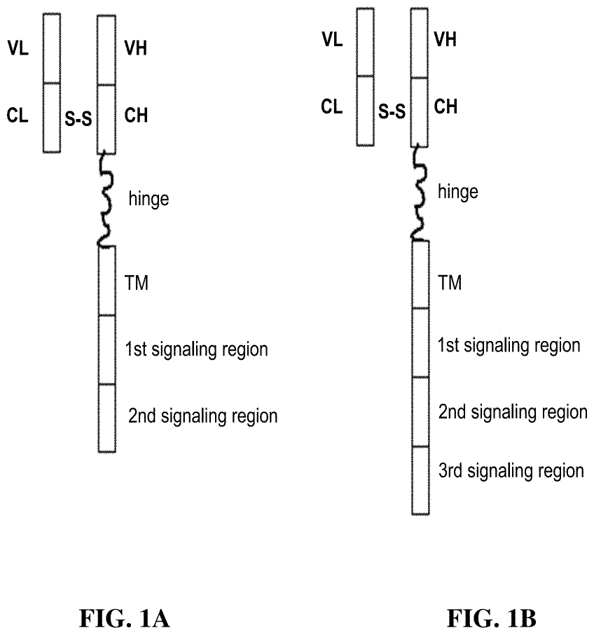

The present disclosure provides a structure for a DAR (dimeric antigen receptor) construct having a first polypeptide chain and a second polypeptide chain, wherein the first polypeptide chain comprises a heavy chain variable region of an antibody and the second polypeptide chain comprises a light chain variable region of an antibody, where the first polypeptide chain is linked to the second polypeptide chain by one or a plurality of disulfide bonds at regions outside of a transduced cell when both the first polypeptide chain and the second polypeptide chain are expressed by a same cell. In some embodiments, a DAR construct comprises a first polypeptide chain comprising, in sequence, an antibody heavy chain with a variable domain region and a CH1 region, a hinge region, a transmembrane region, and an intracellular region having 2-5 signaling domains, and a second polypeptide chain comprising an antibody light chain variable domain region (kappa (K) or lambda (L)) with a corresponding CLICK region, wherein the CH1 and CL/CK regions in each first and second polypeptide chains are linked with one or two disulfide bonds (e.g., see FIGS. 1A and B).

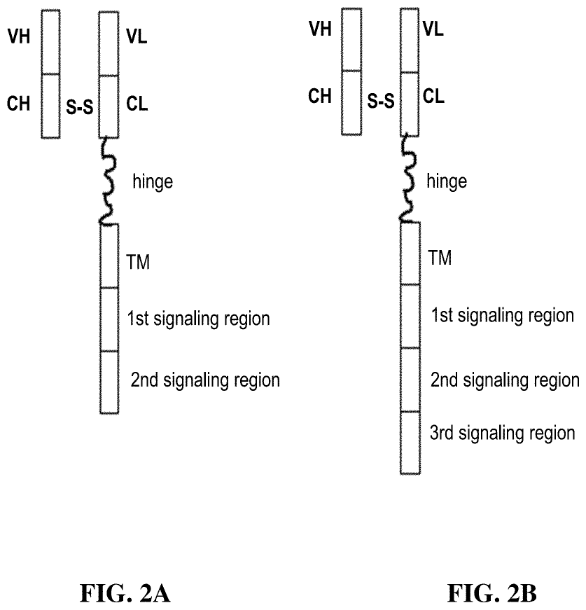

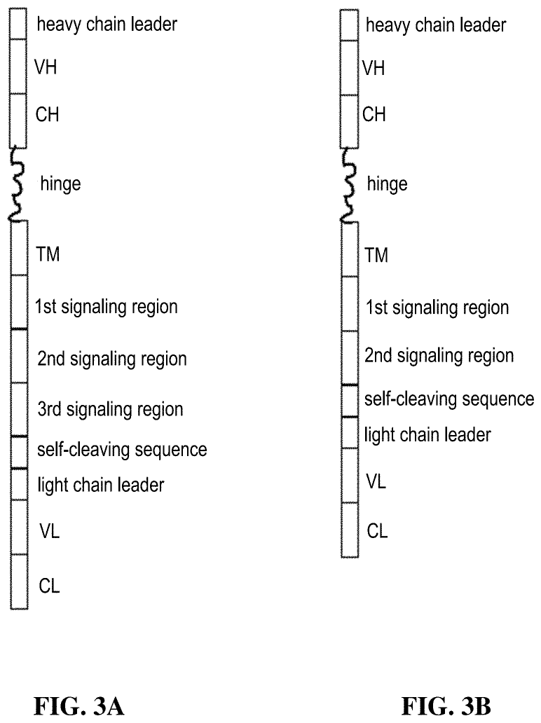

The present disclosure provides a structure for a DAR (dimeric antigen receptor) construct having a first polypeptide chain and a second polypeptide chain, wherein the first polypeptide chain comprises a light chain variable region of an antibody and the second polypeptide chain comprises a heavy chain variable region of an antibody, wherein the first polypeptide chain is linked to the second polypeptide chain by one or a plurality of disulfide bonds at regions outside of a transduced cell when both the first polypeptide chain and the second polypeptide chain are expressed by a same cell. In some embodiments, a DAR construct comprises a first polypeptide chain comprising, in sequence, an antibody light chain with a variable domain region (kappa (K) or lambda (L)) with a corresponding CL/CK region, a hinge region, a transmembrane region, and an intracellular region having 2-5 signaling domains, and a second polypeptide chain comprising, and an antibody heavy chain variable domain region and a CH1 region, wherein the CL/CK and CH1 regions in each first and second polypeptide chains are linked with one or two disulfide bonds (e.g., see FIGS. 2A and B).

In one embodiment, the DAR construct comprises an antibody heavy chain variable region and an antibody light chain variable region on separate polypeptide chains, wherein the heavy chain variable region and the light chain variable region form an antigen binding domain.

In one embodiment, the hinge region is about 10 to about 100 amino acids in length. In one embodiment, the hinge region is independently selected from the group consisting of a CD8 hinge region or a fragment thereof, a CD8α hinge region or a fragment thereof, a hinge region of an antibody (IgG, IgA, IgM, IgE, or IgD) joining the constant domains CH1 and CH2 of an antibody. The hinge region can be derived from an antibody and may or may not comprise one or more constant regions of the antibody.

In one embodiment, the transmembrane domain can be derived from a membrane protein sequence region selected from the group consisting of CD8α, CD80, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD40L/CD154, VEGFR2, FAS, and FGFR2B.

In some embodiments, the signaling region is selected from the group consisting of signaling regions from CD3-zeta chain, 4-1BB, CD28, CD27, OX40, CD30, CD40, PD-1, ICOS, lymph oocyte function-associated antigen-1 (LFA-1), CD2, CD7, LIGHT, NKG2C, B7-H3, GITR (TNFRSF18), DR3 (TNFRSF25), TNFR2, CD226, and combinations thereof.

In some embodiments, a general design of a dimeric antigen receptor includes a first polypeptide chain and a second polypeptide chain, wherein the first polypeptide chain comprises an antigen binding region connected to a dimerization region, connected to a hinge region, connected to a transmembrane region, and connected to one or a plurality of intracellular sequence region(s), and wherein the second polypeptide chain comprises an antigen binding domain and a dimerization domain. In some embodiments, the antigen binding domain on one or both of the first and the second polypeptide chains is selected from the group consisting of a heavy chain variable region, a light chain variable region, an extracellular region of a cytokine receptor, a single domain antibody, and combinations thereof. In some embodiments, the dimerization domain on one or both of the first and second polypeptide chains is selected from the group consisting of a kappa light chain constant region, a lambda light chain constant region, a leucine zipper, myc-max components, and combinations thereof. In FIGS. 1A-B and 2A-B, the “S—S” represents any chemical bond or association that results in dimerization of the first and second polypeptide chains, including disulfide bond, leucine zipper or myc-max components.

The present disclosure provides dimeric antigen receptors (DAR) constructs where the first polypeptide chain carries the heavy chain variable (VH) and heavy chain constant regions (CH), and the second polypeptide chain carries the light chain variable (VL) and light chain constant regions (CL) (e.g., FIGS. 1A and B). In one embodiment, the dimeric antigen receptors (DAR) construct comprises: (a) a first polypeptide chain comprising five regions ordered from the amino terminus to the carboxyl terminus: (i) an antibody heavy chain variable region (VH), (ii) an antibody heavy chain constant region (CH), (iii) an optional hinge region, (iv) a transmembrane region (TM), and (v) an intracellular region; (b) a second polypeptide chain comprising two regions ordered from the amino terminus to the carboxyl terminus: (i) an antibody light chain variable region (VL) (e.g., kappa or lambda), and (ii) an antibody light chain constant region (CL).

The present disclosure provides dimeric antigen receptors (DAR) constructs where the first polypeptide chain carries the light chain variable (VL) and light chain constant regions (CL), and the second polypeptide chain carries the heavy chain variable (VH) and heavy chain constant regions (CH) (e.g., FIGS. 2A and B). In one embodiment, the dimeric antigen receptors (DAR) constructs comprises (a) a first polypeptide chain comprising five regions ordered from the amino terminus to the carboxyl terminus: (i) an antibody light chain variable region (VL), (ii) an antibody light chain constant region (CL), (iii) an optional hinge region, (iv) a transmembrane region (TM), and (v) an intracellular region; (b) a second polypeptide chain comprising two regions ordered from the amino terminus to the carboxyl terminus: (i) an antibody heavy chain variable region (VH), and (ii) an antibody heavy chain constant region (CH1).

In one embodiment, for the dimeric antigen receptors shown in FIGS. 1A and B, and 2A and B, the antibody heavy chain constant region (CH1) and the antibody light chain constant region (CL) can dimerize to form a dimerization domain. In one embodiment, the antibody heavy chain constant region and the antibody light chain constant region dimerize via one or two disulfide bonds.

In one embodiment, for the dimeric antigen receptors shown in FIGS. 1A and B, and in FIGS. 2A and B, the antibody heavy chain variable region (VH) and the antibody light chain variable region (VL) associate with each other to form an antigen binding domain. For example, the antibody heavy chain variable region and the antibody light chain variable region associate with each other when the antibody heavy chain constant region and the antibody light chain constant region dimerize.

In one embodiment, for the dimeric antigen receptors shown in FIGS. 1A and B, and 2A and B, the antigen binding domain, which is formed from the antibody heavy chain variable region and the antibody light chain variable region, binds a target antigen.

In one embodiment, for the dimeric antigen receptors shown in FIGS. 1A and B, and 2A and B, the antibody heavy chain variable region and the antibody light chain variable region are fully human antibody regions, humanized antibody region, or chimeric antibody regions.

In one embodiment, for the dimeric antigen receptors shown in FIGS. 1A and B, and 2A and B, the hinge region is about 10 to about 100 amino acids in length. In one embodiment, the hinge region comprises a hinge region or a fragment thereof from an antibody (e.g., IgG, IgA, IgM, IgE, or IgD). In one embodiment, the hinge region comprises a CD8 (e.g., CD8α) and/or CD28 hinge region or a fragment thereof. In one embodiment, the hinge region comprises a CPPC (SEQ ID NO: 61) or SPPC (SEQ ID NO: 62) amino acid sequence. In one embodiment, the hinge region comprises both CD8 and CD28 hinge sequences (e.g., long hinge region), only CD8 sequence (short hinge) or only CD28 hinge sequence (e.g., short hinge region). In one embodiment, any of the dimeric antigen receptors shown in FIG. 1A or B, or FIG. 2A or B, lack a hinge region.

In one embodiment, for the dimeric antigen receptors shown in FIGS. 1A and B, and 2A and B, the transmembrane regions of the first and second polypeptide chains can be independently derived from CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD40L/CD154, VEGFR2, FAS, and FGFR2B.

In one embodiment, for the dimeric antigen receptors shown in FIGS. 1A and B, and 2A and B, the intracellular region of the first polypeptide comprises intracellular co-stimulatory and/or signaling sequences in any order and of any combination of 2-5 intracellular sequences from 4-1BB, CD3zeta, CD28, CD27, OX40, CD30, CD40, PD-1, ICOS, lymphocyte function-associated antigen-1 (LFA-1), CD2, CD7, LIGHT, NKG2C, B7-H3, GITR (TNFRSF18), DR3 (TNFRSF25), TNFR2, CD226, and combinations thereof. In one embodiment, the intracellular region comprises any one or any combination of two or more of CD28, 4-1BB and/or CD3-zeta intracellular sequences. In one embodiment, the intracellular region comprises CD28 co-stimulatory and CD3-zeta intracellular signaling sequences, or 4-1BB co-stimulatory and CD3-zeta intracellular signaling sequences. In one embodiment, the CD3-zeta portion of the intracellular signaling region comprises ITAM (immunoreceptor tyrosine-based activation motif) motifs 1, 2 and 3 (e.g., long CD3-zeta). In one embodiment, the CD3-zeta portion of the intracellular signaling region comprises only one of the ITAM motifs such as only ITAM 1, 2 or 3 (e.g., short CD3-zeta).

In some exemplary embodiments, a DAR includes a first polypeptide chain that comprises, from the N terminus to the C terminus, an antibody heavy chain variable region followed by an antibody constant region CH1 domain, a hinge region, a transmembrane domain, and two intracellular domains and a second polypeptide that includes an antibody light chain variable region followed by an antibody light chain constant (CL) domain. Alternatively, the DAR can include includes a first polypeptide chain that comprises, from the N terminus to the C terminus, an antibody light chain variable region followed by a light chain antibody constant region (CL), a hinge region, a transmembrane domain, and two intracellular domains and a second polypeptide that includes an antibody heavy chain variable region followed by an antibody heavy chain constant CH1 domain.

The heavy and light chain variable regions are derived from the same antigen-binding antibody, where the antigen can be, for example, CD20. In some embodiments the heavy chain variable region has at least at least 95%, 96%, 97%, 98%, or 99% amino acid sequence identity to SEQ ID NO:3 and includes the CDR regions of SEQ ID NO:3 and the light chain variable region has at least 95%, 96%, 97%, 98%, or 99% amino acid sequence identity to SEQ ID NO:11 and includes the CDR regions of SEQ ID NO:11. The heavy chain constant regions (CH1) can have at least 95%, 96%, 97%, 98%, or 99% amino acid sequence identity to SEQ ID NO:4. The light chain constant region (CL) can have at least 95%, 96%, 97%, 98%, or 99% amino acid sequence identity to SEQ ID NO:12.

The hinge region can be, for example, a CD28 hinge region or an amino acid sequence having at least 95%, 96%, 97%, 98%, or 99% amino acid sequence identity to the hinge region of CD28 (SEQ ID NO:5), and the transmembrane domain can be a CD28 transmembrane region or an amino acid sequence having at least 95%, 96%, 97%, 98%, or 99% amino acid sequence identity to the transmembrane domain of CD28 (SEQ ID NO:6). The two intracellular domains can be, for example, a 4-1BB intracellular domain or an amino acid sequence having at least 95%, 96%, 97%, 98%, or 99% amino acid sequence identity to the intracellular domain of 4-1BB (SEQ ID NO:7), followed by a CD3zeta intracellular domain that includes ITAM 3 having the sequence of SEQ ID NO:8, or an amino acid sequence having at least 95%, 96%, 97%, 98%, or 99% amino acid sequence identity thereto. The DAR can be a CD20 DAR, that is, a dimeric receptor whose ligand is CD20 (e.g., SEQ ID NO:1). The DAR can be encoded by at least one nucleic acid molecule, for example, a single nucleic acid molecule that includes a first and a second open reading frame for the first and second polypeptides, respectively, where the two open reading frames may each be operably linked to a promoter or joined by an IRES for translation as two polypeptides. Alternatively, as exemplified herein, a continuous open reading frame can join the two polypeptide-encoding sequences by a 2A sequence so that they are produced as two polypeptides. The two polypeptides can also be encoded on two different DNA molecules, where each transgene encoding is operably linked to its own promoter. A nucleic acid construct encoding a first polypeptide and a second polypeptide can include a sequence encoding a signal peptide upstream of and in the same reading frame as the variable antibody region-encoding portion of the construct(s) so that the first and second polypeptides are directed to the membrane (first polypeptide) or secreted (second polypeptide).

One of skill will recognize and as disclosed herein there are many alternatives that may be used for signal peptides, hinge regions, and transmembrane regions, which can be from proteins known in the art or modified therefrom using knowledge of conserved structural features of these protein regions. In particular embodiments, a CD20 DAR as provided herein comprises a first polypeptide having at least 95%, 96%, 97%, 98%, or 99% amino acid sequence identity to SEQ ID NO:14 and a second polypeptide having at least 95%, 96%, 97%, 98%, or 99% amino acid sequence identity to SEQ ID NO:15. In one embodiment, a CD20 DAR as provided herein comprises a first polypeptide having the amino acid sequence of SEQ ID NO:14 and a second polypeptide having the amino acid sequence identity of SEQ ID NO:15.

### Cells Expressing Dimeric Antigen Receptors (DARs)

Also provided herein are transgenic cells that include nucleic acids encoding a DAR as described herein. The transgenic cell can include nucleic acids that encode the two polypeptides of the DAR in any configuration that allows for the production of two polypeptides by the cell. Each polypeptide encoding sequence has at its 5′ end a sequence encoding a signal peptide that directs membrane insertion of the first polypeptide and secretion of the second polypeptide.

In some examples, the transgenic host cell can include a nucleic acid construct in which the two polypeptides are produced from a single open reading frame, where, for example, the sequence encoding the first polypeptide is joined to the nucleotide sequence encoding the second polypeptide by a sequence encoding a 2A “self-cleaving” peptide that allows for the production of separate polypeptides. In this configuration, the sequence encoding the precursor polypeptide is operably linked to a single promoter. In an alternative configuration, open reading frames encoding a first and second DAR polypeptide can be joined by an IRES. Alternatively, each polypeptide can be encoded by a separate open reading frame operably linked to a separate promoter.

Nucleic acid molecules encoding DAR polypeptides are also provided herein. In some embodiments, one or more nucleic acid molecules is provided that includes a first open reading frame encoding a first polypeptide as disclosed herein, and a second open reading frame encoding a second polypeptide as disclosed herein. For example, the first and second open reading frames can be provided on one or two nucleic acid molecules, such as one or two DNA or RNA fragments or one or more nucleic acid vectors. One or both of the open reading frames can be operable linked to a promoter. A promoter operably linked to an open reading frame encoding at least one of a first DAR polypeptide and a second DAR polypeptide is preferably functional in a mammalian cell, such as a human cell. Examples of mammalian promoters that may be operably linked to a first or second DAR polypeptide gene include, without limitation, a CMV promoter, an EF1α promoter, an HTLV promoter, an EF1α/HTLV hybrid promoter, and a JeT promoter.

Transgenic host cells can be prepared by transducing host cells (such as but not limited to PBMCs or T cells) with a retroviral vector carrying a nucleic acid encoding a CAR or DAR construct. The transduction can be performed essentially as described in Ma et al., 2004 The Prostate 61:12-25; and Ma et al., The Prostate 74(3):286-296, 2014 (the disclosures of which are incorporated by reference herein in their entireties). The retroviral vector can be transfected into a Phoenix-Eco cell line (ATCC) using FuGene reagent (Promega, Madison, Wis.) to produce Ecotropic retrovirus, then harvest transient viral supernatant (Ecotropic virus) can be used to transduce PG13 packaging cells with Gal-V envelope to produce retrovirus to infect human cells. Viral supernatant from the PG13 cells can be used to transduce activated T cells (or PBMCs) two to three days after CD3 or CD3/CD28 activation. Activated human T cells can be prepared by activating normal healthy donor peripheral blood mononuclear cells (PBMC) with 100 ng/ml mouse anti-human CD3 antibody OKT3 (Orth Biotech, Rartian, N.J.) or anti-CD3, anti-CD28 TransAct (Miltenyi Biotech, German) as manufacturer's manual and 300-1000 U/ml IL2 in AIM-V growth medium (GIBCO-Thermo Fisher scientific, Waltham, Mass.) supplemented with 5% FBS for two days. Approximately 5×10⁶ activated human T cells can be transduced in a 10 ug/ml retronectin (Takara Bio USA) pre-coated 6-well plate with 3 ml viral supernatant and centrifuged at 1000 g for about 1 hour at approximately 32° C. After transduction, the transduced T cells can be expanded in AIM-V growth medium supplemented with 5% FBS and 300-1000 U/ml IL2.

Transgenic host cells can be prepared using non-viral methods, including well-known designer nucleases including zinc finger nucleases, TALENS or CRISPR/Cas. A transgene can be introduced into a host cell's genome using genome editing technologies such as zinc finger nuclease. A zinc finger nuclease includes a pair of chimeric proteins each containing a non-specific endonuclease domain of a restriction endonuclease (e.g., FokI) fused to a DNA-binding domain from an engineered zinc finger motif. The DNA-binding domain can be engineered to bind a specific sequence in the host's genome and the endonuclease domain makes a double-stranded cut. The donor DNA carries the transgene, for example any of the nucleic acids encoding a CAR or DAR construct described herein, and flanking sequences that are homologous to the regions on either side of the intended insertion site in the host cell's genome. The host cell's DNA repair machinery enables precise insertion of the transgene by homologous DNA repair. Transgenic mammalian host cells have been prepared using zinc finger nucleases (U.S. Pat. Nos. 9,597,357, 9,616,090, 9,816,074 and 8,945,868). A transgenic host cell can be prepared using TALEN (Transcription Activator-Like Effector Nucleases) which are similar to zinc finger nucleases in that they include a non-specific endonuclease domain fused to a DNA-binding domain which can deliver precise transgene insertion. Like zinc finger nucleases, TALEN also introduce a double-strand cut into the host's DNA. Transgenic host cells can be prepared using CRISPR (Clustered Regularly Interspaced Short Palindromic Repeats). CRISPR employs a Cas endonuclease coupled to a guide RNA for target specific donor DNA integration. The guide RNA includes a conserved multi-nucleotide containing protospacer adjacent motif (PAM) sequence upstream of the gRNA-binding region in the target DNA and hybridizes to the host cell target site where the Cas endonuclease cleaves the double-stranded target DNA. The guide RNA can be designed to hybridize to a specific target site. Similar to zinc finger nuclease and TALEN, the CRISPR/Cas system can be used to introduce site specific insertion of donor DNA having flanking sequences that have homology to the insertion site. Examples of CRISPR/Cas systems used to modify genomes are described for example in U.S. Pat. Nos. 8,697,359, 10,000,772, 9,790,490, and U. S. Patent Application Publication No. US 2018/0346927.

As exemplified herein, CRISPR/Cas methods that simultaneous knock out an endogenous gene of the host cells when an exogenous construct is inserted at the locus can be employed (see, for example, US 2020/0224160 and WO 2020/185867, both of which are incorporated by reference herein in their entireties). Transgenic host cells produced by such methods can incorporate a nucleic acid molecule encoding DAR polypeptides while losing expression of, for example, the TRAC (TCR alpha chain) gene. The transgenic host cells can be T cells, for example, can be CD3+ cells (expressing the T cell receptor) isolated from PBMCs prior to transfection. Further, following transfection with a DAR construct and Cas RNP targeting the TRAC locus, the transfected culture can be expanded and then CD3+ cells (i.e., cells that express the T cell receptor) can be depleted, for example, using magnetic beads conjugated to a CD3 antibody, resulting in cultures enriched for cells in which the TRAC gene has been knocked out by incorporation of the DAR construct.

Provided herein are cell cultures wherein at least 5%, at least 10%, at least 15%, at least 20%, at least 25%, at least 30%, at least 35%, at least 40%, at least 45%, or at least 50% of the cells of the culture express a DAR construct. The cell cultures can be T cell cultures, for example, primary T cell cultures and can be cultures of primary human T cells. In various embodiments the T cell cultures include less than 10%, less than 8%, less than 7%, less than 5%, less than 3%, less than 2%, less than 1.5%, less than 1%, or less than 0.5% CD3+ cells, e.g., less than 10%, less than 8%, less than 7%, less than 5%, less than 3%, less than 2%, less than 1.5%, less than 1%, or less than 0.5% of the cells of the culture express the endogenous T cell receptor. Provided herein is a population of DAR-T cells in which at least 10%, at least 15%, at least 20%, at least 25%, at least 30%, at least 35%, at least 40%, at least 45%, or at least 50% of the cells of the culture express a DAR construct and less than 8%, less than 7%, less than 5%, less than 3%, less than 2%, less than 1.5%, less than 1%, or less than 0.5% CD3+ cells, e.g., less than 10%, less than 8%, less than 7%, less than 5%, less than 3%, less than 2%, less than 1.5%, less than 1%, or less than 0.5% of the cells of the culture express the endogenous T cell receptor and a pharmaceutical composition comprising such a cell population. The cell population can be provided as a composition formulated for intravenous infusion or injection, for example.

In further embodiments, transgenic host cells can be prepared using zinc finger nuclease, TALEN or CRISPR/Cas system, and the host target site can be a TRAC gene (T Cell Receptor Alpha Constant). The donor DNA can include for example any of the nucleic acids encoding a CAR or DAR construct described herein. Electroporation, nucleofection or lipofection can be used to co-deliver into the host cell the donor DNA with the zinc finger nuclease, TALEN or CRISPR/Cas system. Other methods of integrating a construct encoding a DAR or CAR construct can include using transposases such as Sleeping Beauty or Piggy Back.

In various embodiments, a host cell can be introduced with an expression vector or nucleic acid fragment in which a promoter is operably linked to a nucleic acid sequence encoding a DAR thereby generating a transfected/transformed host cell which is cultured under conditions suitable for expression of the DAR by the transfected/transformed host cell.

Typically, a host cell is a cultured cell that can be transformed or transfected with a polypeptide-encoding nucleic acid, which can then be expressed in the host cell. The phrase “transgenic host cell” or “recombinant host cell” can be used to denote a host cell that has been introduced (e.g., transduced, transformed, or transfected) with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein. A host cell, or a population of host cells, harboring a vector (e.g., an expression vector) operably linked to at least one nucleic acid encoding one or more polypeptides that comprise a dimeric antigen receptor (DAR) or antigen-binding portions thereof are described herein.

In various embodiments the host cell or the population of host cells can comprise T lymphocytes (e.g., T cells, regulatory T cells, gamma-delta T cells, and cytotoxic T cells), NK (natural killer) cells, macrophages, dendritic cells, mast cells, eosinophils, B lymphocytes, monocytes. In some embodiments, the NK cells comprise cord blood-derived NK cells, or placental derived NK cells. A population of host cells can comprise human T lymphocytes or human NK cells, for example, can be primary human T cells or NK cells. In various exemplary embodiments provided herein, primary human DAR-T cells, i.e., primary human T cells transfected with a DAR construct and expressing a DAR, are provided. The host cells provided herein can be a cell population where the population of host cells has been positively selected (for example, selected by magnetic beads conjugated to a binding partner or by the use of other capture reagents of formats and/or by flow cytometry) and/or may be a population of host cells from which some cell types have been removed or depleted (subtracted) for example, by magnetic bead capture, flow cytometry, or other methods. For example, the population of host cells may be selected for the expression of the T cell receptor or may be depleted of cells expressing the T cell receptor (e.g., by use of an antibody binding CD3). The cells may be selected or enriched by use of a binding partner that is bound by the expression of the construct (DAR or CAR) transfected into cells. Further, a population of host cells may be selectively expanded, for example, by culturing in the presence of a binding partner for the CAR or DAR expressed by the transfected cells of the population (e.g., a CD20 CAR or DAR), or in the presence of cells that express the binding partner (e.g., CD20).

Cell populations that comprise transgenic cells expressing a DAR as provided herein can be provided as pharmaceutical compositions, for example, for injection or infusion. In some embodiments, the cells are T cells or NK cells. In some embodiments, the cells are T cells that do not express an endogenous T cell receptor. In some embodiments, a pharmaceutical preparation includes a population of primary T cells, such as human primary T cells, where at least 20% of the cells of the population express a DAR construct and less than 5%, less than 2%, less than 1%, or less than 0.5% of the cells of the population express an endogenous T cell receptor.

A population of DAR-expressing cells, such as any described herein, for example DAR-T cells, can be provided for infusion (e.g., intravenous or intraarterial infusion) or injection (e.g., one or more intravenous, intratumoral, or subcutaneous injections). The cell formulation can be frozen for storage and shipping and can optionally provide cells for multiple treatments with the same cell preparation. The cells can be packaged as a product or kit in vials, bags, or tubes, for example. Instructions (e.g., written instructions) may be provided on the use of the cells.

Provided herein are methods of treating cancer comprising administering to a subject a therapeutically-effective amount of a population of cells as provided herein that express a DAR, such as a CD20 DAR, such as any described herein. The cells may be T cells and at least 10% of the cell population can express the DAR. In some embodiments the cancer is a hematological cancer.

The cells can be administered for at least about 10 seconds, 30 seconds, 1 minute, 10 minutes, 30 minutes, 1 hour, 2 hours, 3 hours, 4 hours, 5 hours, 10 hours, 12 hours, 24 hours in a single dosing. Cells can be administered in a single dose or in multiple doses over minutes, hours, days, weeks, or months. A population of DAR-T expressing cells as described herein can be administered before, during, or after the occurrence of a disease or condition, and the timing of administering a pharmaceutical composition containing the DAR expressing cell population can vary. The initial administration can be via any route practical, such as by any route described herein using any formulation described herein. In some examples, the administration is an intravenous administration. In some embodiments, one or multiple dosages of the DAR T-cell population can be administered after the onset of a hematological cancer and optionally for a length of time necessary for the treatment of the disease.

### Sequences

The following exemplary sequences are disclosed.

Homo sapiens
CD20 antigen (UniProtKB P11836 - gene MS4A1)
SEQ ID NO: 1
MTTPRNSVNGTFPAEPMKGPIAMQSGPKPLFRRMSSLVGPTQSFFMRESKTLGAVQIMNG
LFHIALGGLLMIPAGIYAPICVTVWYPLWGGIMYIISGSLLAATEKNSRKCLVKGKMIMN
SLSLFAAISGMILSIMDILNIKISHFLKMESLNFIRAHTPYINIYNCEPANPSEKNSPST
QYCYSIQSLFLGILSVMLIFAFFQELVIAGIVENEWKRTCSRPKSNIVLLSAEEKKEQTI
EIKEEVVGLTETSSQPKNEEDIEIIPIQEEEEEETETNFPEPPQDQESSPIENDSSP
Mus musculus
Heavy chain leader peptide
SEQ ID NO: 2
MEWSWVFLFFLSVTTGVHS
Anti-CD20 heavy chain variable region:
SEQ ID NO: 3
QVQLQQPGAELVKPGASVKMSCKASGYTFTSYNMHWVKQTPGRGLEWIGAIYPGNGDTSYNQKF
KGKATLTADKSSSTAYMQLSSLTSEDSAVYYCARSTYYGGDWYFNVWGAGTTVTVSA
Anti-CD20 heavy chain constant region (CH1)
SEQ ID NO: 4
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHT
CD28 hinge
SEQ ID NO: 5
PRKIEVMYPPPYLDNEKSNGTIIHVKGKHLCPSPLFPGPSKP
CD28 transmembrane domain
SEQ ID NO: 6
FWVLVVVGGVLACYSLLVTVAFIIFWV
4-1BB intracellular costimulatory sequence
SEQ ID NO: 7
KRGRKKLLYIFKQPFMRPVQTTQEEDGCSCRFPEEEEGGCEL
CD3zeta intracellular signaling region, ITAM3 only
SEQ ID NO: 8
RVKFSRSADKGERRRGKGHDGLYQGLSTATKDTYDALHMQALPPR
Thosea asigna virus
T2A self-cleaving sequence
SEQ ID NO: 9
GSGEGRGSLLTCGDVEENPGP
Light chain leader peptide
SEQ ID NO: 10
MSVPTQVLGLLLLWLTDARC
Anti-CD20 light chain variable region
SEQ ID NO: 11
QIVLSQSPAILSASPGEKVTMTCRASSSVSYIHWFQQKPGSSPKPWIYATSNLASGVPVRFSGS
GSGTSYSLTISRVEAEDAATYYCQQWTSNPPTFGGGTKLEIK
Anti-CD20
light chain constant region (kappa)
SEQ ID NO: 12
RTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
Anti-CD20 DAR precursor polypeptide:
SEQ ID NO: 13
MEWSWVFLFFLSVTTGVHSQVQLQQPGAELVKPGASVKMSCKASGYTFTSYNMHWVKQTPGRGL
EWIGAIYPGNGDTSYNQKFKGKATLTADKSSSTAYMQLSSLTSEDSAVYYCARSTYYGGDWYFN
VWGAGTTVTVSAASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDKTHTPRKIEVMY
PPPYLDNEKSNGTIIHVKGKHLCPSPLFPGPSKPFWVLVVVGGVLACYSLLVTVAFIIFWVKRG
RKKLLYIFKQPFMRPVQTTQEEDGCSCRFPEEEEGGCELRVKFSRSADKGERRRGKGHDGLYQG
LSTATKDTYDALHMQALPPRGSGEGRGSLLTCGDVEENPGPMSVPTQVLGLLLLWLTDARCQIV
LSQSPAILSASPGEKVTMTCRASSSVSYIHWFQQKPGSSPKPWIYATSNLASGVPVRFSGSGSG
TSYSLTISRVEAEDAATYYCQQWTSNPPTFGGGTKLEIKRTVAAPSVFIFPPSDEQLKSGTASV
VCLLNNFYPREAKVQWKVDNALQSGNSQESVTEQDSKDSTYSLSSTLTLSKADYEKHKVYACEV
THQGLSSPVTKSFNRGEC
Protein
Artificial
Anti-CD20 DAR 1^{st} polypeptide chain
SEQ ID NO: 14
QVQLQQPGAELVKPGASVKMSCKASGYTFTSYNMHWVKQTPGRGLEWIGAIYPGNGDTSYNQKF
KGKATLTADKSSSTAYMQLSSLTSEDSAVYYCARSTYYGGDWYFNVWGAGTTVTVSAASTKGPS
VFPLAPSSKSTSGGTAALGCLVKDYFPEPVTVSWNSGALTSGVHTFPAVLQSSGLYSLSSVVTV
PSSSLGTQTYICNVNHKPSNTKVDKRVEPKSCDKTHTPRKIEVMYPPPYLDNEKSNGTIIHVKG
KHLCPSPLFPGPSKPFWVLVVVGGVLACYSLLVTVAFIIFWVKRGRKKLLYIFKQPFMRPVQTT
QEEDGCSCRFPEEEEGGCELRVKFSRSADKGERRRGKGHDGLYQGLSTATKDTYDALHMQALPR
R
Protein
Artificial
Anti-CD20 DAR 2^{nd} polypeptide chain
SEQ ID NO: 15
QIVLSQSPAILSASPGEKVTMTCRASSSVSYIHWFQQKPGSSPKPWIYATSNLASGVPVRFSGS
GSGTSYSLTISRVEAEDAATYYCQQWTSNPPTFGGGTKLEIKRTVAAPSVFIFPPSDEQLKSGT
ASVVCLLNNFYPREAKVQWKVDNALQSGNSQESVTEQDSKDSTYSLSSTLTLSKADYEKHKVYA
CEVTHQGLSSPVTKSFNRGEC
DNA
Artificial
sequence encoding the anti-CD20 DAR
SEQ ID NO: 16
ATGGAGTGGAGCTGGGTGTTTCTGTTCTTCCTCTCCGTCACAACCGGCGTGCATAGCCAAGTGC
AATTGCAGCAGCCCGGTGCCGAACTCGTGAAACCAGGAGCAAGCGTAAAGATGTCCTGTAAGGC
ATCAGGTTATACCTTTACCAGCTACAACATGCACTGGGTGAAACAAACGCCGGGGCGGGGCCTC
GAATGGATAGGCGCGATATATCCCGGAAATGGCGATACCAGTTACAATCAGAAGTTCAAAGGCA
AAGCGACACTGACAGCTGATAAGTCTTCAAGCACCGCCTATATGCAACTTTCTAGCCTGACCAG
CGAAGACTCCGCCGTTTATTACTGTGCTCGGTCCACATACTACGGAGGCGATTGGTACTTTAAT
GTGTGGGGTGCGGGCACCACTGTCACTGTATCAGCGGCTTCCACCAAGGGCCCCTCCGTGTTCC
CTCTGGCCCCCAGCAGCAAGAGCACATCCGGAGGCACCGCCGCCCTCGGATGTCTGGTGAAGGA
CTACTTCCCCGAGCCTGTCACCGTGTCCTGGAATAGCGGCGCCCTCACCTCCGGCGTGCACACC
TTCCCCGCTGTCCTGCAGTCCTCCGGACTGTACAGCCTGTCCTCCGTCGTGACCGTGCCTAGCT
CCTCCCTCGGCACCCAGACCTACATCTGCAACGTGAACCACAAGCCTTCCAACACAAAGGTGGA
CAAACGGGTGGAGCCCAAGTCCTGCGACAAAACCCACACCCCCAGAAAGATAGAGGTGATGTAC
CCTCCCCCCTACTTGGACAACGAAAAGTCTAATGGCACTATCATTCACGTAAAGGGCAAACACC
TTTGTCCAAGTCCTTTGTTCCCAGGCCCATCTAAGCCGTTCTGGGTACTCGTGGTTGTGGGGGG
CGTGCTCGCTTGTTACTCACTGCTGGTGACGGTGGCCTTTATTATTTTCTGGGTTAAACGGGGC
AGAAAGAAACTCCTGTATATATTCAAACAACCATTTATGAGACGAGTACAAACTACTCAAGAGG
AAGATGGCTGTAGCTGCCGATTTCCAGAAGAGGAGGAAGGAGGATGTGAACTGAGGGTAAAATT
TAGCAGGTCTGCAGATAAAGGGGAGAGGAGACGCGGGAAGGGCCATGATGGACTGTATCAGGGA
CTTTCCACAGCCACCAAGGACACCTATGACGCTCTCCACATGCAGGCCCTGCCCCCTCGCGGAA
GCGGAGAGGGCAGAGGAAGTCTGCTAACATGCGGTGACGTCGAGGAGAATCCTGGACCTATGTC
CGTCCCTACCCAGGTGCTGGGCCTGCTGCTGCTGTGGCTGACCGATGCTAGATGCCAGATAGTC
CTGAGCCAATCACCGGCCATCTTGTCTGCCTCTCCTGGCGAAAAGGTGACGATGACTTGCAGAG
CCAGTAGCTCTGTAAGCTATATACACTGGTTCCAGCAAAAACCGGGCTCTTCTCCGAAGCCGTG
GATATACGCAACTTCAAACCTGGCGTCTGGGGTTCCTGTAAGGTTTAGCGGCAGCGGTTCAGGC
ACGAGCTACAGCCTTACTATCTCCCGGGTTGAGGCTGAAGATGCAGCCACATACTACTGTCAGC
AGTGGACTTCAAATCCACCTACATTCGGGGGAGGCACGAAGCTGGAGATTAAACGAACCGTTGC
GGCGCCTAGTGTGTTCATATTCCCGCCGTCTGATGAACAACTCAAGTCTGGAACGGCAAGTGTG
GTGTGTCTCCTGAATAATTTTTATCCTAGGGAAGCAAAGGTGCAGTGGAAAGTCGATAACGCAT
TGCAAAGCGGTAACAGTCAAGAATCTGTAACTGAACAAGATTCTAAAGATTCTACCTAGAGTCT
CTCCTCCACATTGACCCTGTCAAAAGCAGATTATGAGAAGCACAAGGTGTACGCATGTGAGGTA
ACACATCAAGGACTCAGCAGCCCAGTTACAAAAAGTTTCAATCGCGGGGAATGT
DNA
Artificial
sequence encoding the anti-CD20 CAR
SEQ ID NO: 17
ATGGAGTGGAGCTGGGTCTTTCTGTTCTTTTTAAGCGTGACCACCGGAGTGCACTCCCAGATAG
TCCTGAGCCAATCACCGGCCATCTTGTCTGCCTCTCCTGGCGAAAAGGTGACGATGACTTGCAG
AGCCAGTAGCTCTGTAAGCTATATACACTGGTTCCAGCAAAAACCGGGCTCTTCTCCGAAGCCG
TGGATATACGCAACTTCAAACCTGGCGTCTGGGGTTCCTGTAAGGTTTAGCGGCAGCGGTTCAG
GCACGAGCTACAGCCTTACTATCTCCCGGGTTGAGGCTGAAGATGCAGCCACATACTACTGTCA
GCAGTGGACTTCAAATCCACCTACATTCGGGGGAGGCACGAAGCTGGAGATTAAAGGCGGCGGC
GGCAGCGGTGGCGGTGGCTCAGGTGGTGGTGGTTCTCAAGTGCAATTGCAGCAGCCCGGTGCCG
AACTCGTGAAACCAGGAGCAAGCGTAAAGATGTCCTGTAAGGCATCAGGTTATACCTTTACCAG
CTACAACATGCACTGGGTGAAACAAACGCCGGGGCGGGGCCTCGAATGGATAGGCGCGATATAT
CCCGGAAATGGCGATACCAGTTACAATCAGAAGTTCAAAGGCAAAGCGACACTGACAGCTGATA
AGTCTTCAAGCACCGCCTATATGCAACTTTCTAGCCTGACCAGCGAAGACTCCGCCGTTTATTA
CTGTGCTCGGTCCACATACTACGGAGGCGATTGGTACTTTAATGTGTGGGGTGCGGGCACCACT
GTCACTGTATCAGCGCCCAGAAAGATAGAGGTGATGTACCCTCCCCCCTACTTGGACAACGAAA
AGTCTAATGGCACTATCATTCACGTAAAGGGCAAACACCTTTGTCCAAGTCCTTTGTTCCCAGG
CCCATCTAAGCCGTTCTGGGTACTCGTGGTTGTGGGGGGCGTGCTCGCTTGTTACTCACTGCTG
GTGACGGTGGCCTTTATTATTTTCTGGGTTAAACGGGGCAGAAAGAAACTCCTGTATATATTCA
AACAACCATTTATGAGACCAGTACAAACTACTCAAGAGGAAGATGGCTGTAGCTGCCGATTTCC
AGAAGAAGAAGAAGGAGGATGTGAACTGAGGGTAAAATTTAGCAGGTCTGCAGATAAAGGGGAG
AGGAGACGCGGGAAGGGCCATGATGGACTGTATCAGGGACTTTCCACAGCCACCAAGGACACCT
ATGACGCTCTCCACATGCAGGCCCTGCCCCCTCGC
Protein
Artificial
Anti-CD20 CAR precursor polypeptide
SEQ ID NO: 18
MEWSWVFLFFLSVTTGVHSQIVLSQSPAILSASPGEKVTMTCRASSSVSYIHWFQQKPGSSPKP
WIYATSNLASGVPVRFSGSGSGTSYSLTISRVEAEDAATYYCQQWTSNPPTFGGGTKLEIKGGG
GSGGGGSGGGGSQVQLQQPGAELVKPGASVKMSCKASGYTFTSYNMHWVKQTPGRGLEWIGAIY
PGNGDTSYNQKFKGKATLTADKSSSTAYMQLSSLTSEDSAVYYCARSTYYGGDWYFNVWGAGTT
VTVSAPRKIEVMYPPPYLDNEKSNGTIIHVKGKHLCPSPLFPGPSKPFWVLVVVGGVLACYSLL
VTVAFIIFWVKRGRKKLLYIFKQPFMRPVQTTQEEDGCSCRFPEEEEGGCELRVKFSRSADKGE
RRRGKGHDGLYQGLSTATKDTYDALHMQALRPR
Artificial
CAR GS linker
SEQ ID NO: 19
GGGGSGGGGSGGGGS
DNA
Artificial
Jet promoter
SEQ ID NO: 20
GAATTCGGGCGGAGTTAGGGCGGAGCCAATCAGCGTGCGCCGTTCCGAAAGTTGCCTTTTATGG
CTGGGCGGAGAATGGGCGGTGAACGCCGATGATTATATAAGGACGCGCCGGGTGTGGCACAGCT
AGTTCCGTCGCAGCCGGGATTTGGGTCGCGGTTCTTGTTTGTGGATCCCTGTGATCGTCACTTG
ACA
DNA
DNA
Homo sapiens
5′ homology arm from exon 1 of TRAC gene, Cas9 target site, 660 nt
SEQ ID NO: 21
GGCACCATATTCATTTTGCAGGTGAAATTCCTGAGATGTAAGGAGCTGCTGTGACTTGCTCAAG
GCCTTATATCGAGTAAACGGTAGTGCTGGGGCTTAGACGCAGGTGTTCTGATTTATAGTTCAAA
ACCTCTATCAATGAGAGAGCAATCTCCTGGTAATGTGATAGATTTCCCAACTTAATGCCAACAT
ACCATAAACCTCCCATTCTGCTAATGCCCAGCCTAAGTTGGGGAGACCACTCCAGATTCCAAGA
TGTACAGTTTGCTTTGCTGGGCCTTTTTCCCATGCCTGCCTTTACTCTGCCAGAGTTATATTGC
TGGGGTTTTGAAGAAGATCCTATTAAATAAAAGAATAAGCAGTATTATTAAGTAGCCCTGCATT
TCAGGTTTCCTTGAGTGGCAGGCCAGGCCTGGCCGTGAACGTTCACTGAAATCATGGCCTCTTG
GCCAAGATTGATAGCTTGTGCCTGTCCCTGAGTCCCAGTCCATCACGAGCAGCTGGTTTCTAAG
ATGCTATTTCCCGTATAAAGCATGAGACCGTGACTTGCCAGCCCCACAGAGCCCCGCCCTTGTC
CATCACTGGCATCTGGACTCCAGCCTGGGTTGGGGCAAAGAGGGAAATGAGATCATGTCCTAAC
CCTGATCCTCTTGTCCCACA
DNA
Homo sapiens
3′ homology arm from exon 1 of TRAC gene, Cas9 target site, 650 nt
SEQ ID NO: 22
GATATCCAGAACCCTGACCCTGCCGTGTACCAGCTGAGAGACTCTAAATCCAGTGACAAGTCTG
TCTGCCTATTCACCGATTTTGATTCTCAAACAAATGTGTCACAAAGTAAGGATTCTGATGTGTA
TATCACAGACAAAACTGTGCTAGACATGAGGTCTATGGACTTCAAGAGCAACAGTGCTGTGGCC
TGGAGCAACAAATCTGAGTTTGCATGTGCAAACGCCTTCAACAACAGCATTATTCCAGAGGACA
CCTTCTTCCCCAGCCCAGGTAAGGGCAGCTTTGGTGCCTTCGCAGGCTGTTTCCTTGCTTCAGG
AATGGCCAGGTTCTGCCCAGAGCTCTGGTCAATGATGTCTAAAACTCCTCTGATTGGTGGTCTC
GGCCTTATCCATTGCCACCAAAACCCTCTTTTTACTAAGAAACAGTGAGCCTTGTTCTGGCAGT
CCAGAGAATGACACGGGAAAAAAGCAGATGAAGAGAAGGTGGCAGGAGAGGGCACGTGGCCCAG
CCTCAGTCTCTCCAACTGAGTTCCTGCCTGCCTGCCTTTGCTCAGACTGTTTGCCCCTTACTGC
TCTTCTAGGCCTCATTCTAAGCCCCTTCTCCAAGTTGCCTCTCCTTATTTCTCCCTGTCTGCCA
AAAAATCTTT
DNA
Homo sapiens
Cas9 target site, TRAC locus
SEQ ID NO: 23
CAGGGTTCTGGATATCTGT
DNA
Homo sapiens
171 bp 5′ homology region, Cas9 target site
SEQ ID NO: 24
ATCACGAGCAGCTGGTTTCTAAGATGCTATTTCCCGTATAAAGCATGAGACCGTGACTTGCCAG
CCCCACAGAGCCCCGCCCTTGTCCATCACTGGCATCTGGACTCCAGCCTGGGTTGGGGCAAAGA
GGGAAATGAGATCATGTCCTAACCCTGATCCTCTTGTCCCACA
DNA
Homo sapiens
161 bp 3’ homology region, Cas9 target site
SEQ ID NO: 25
GATATCCAGAACCCTGACCCTGCCGTGTACCAGCTGAGAGACTCTAAATCCAGTGACAAGTCTG
TCTGCCTATTCACCGATTTTGATTCTCAAACAAATGTGTCACAAAGTAAGGATTCTGATGTGTA
TATCACAGACAAAACTGTGCTAGACATGAGGTC
DNA
Artificial
Forward primer
SEQ ID NO: 26
A*TmC*mA*mCGAGCAGCTGGTTTCT
(*indicates phosphorothioate linkage; mA indicates
2’-O-methyladenosine; mC indicates 2’-O-methylcytidine)
DNA
Artificial
Reverse primer
SEQ ID NO: 27
GACCTCATGTCTAGCACAGTTTTG
DNA
Homo sapiens
5′ exon 1 TRAC gene homology flanking sequence, Cas12a target site, 645 bp
SEQ ID NO: 28
TGTAAGGAGCTGCTGTGACTTGCTCAAGGCCTTATATCGAGTAAACGGTAGTGCTGGGGCTTAG
ACGCAGGTGTTCTGATTTATAGTTCAAAACCTCTATCAATGAGAGAGCAATCTCCTGGTAATGT
GATAGATTTCCCAACTTAATGCCAACATACCATAAACCTCCCATTCTGCTAATGCCGAGCCTAA
GTTGGGGAGACCACTCCAGATTCCAAGATGTACAGTTTGCTTTGCTGGGCCTTTTTCCCATGCC
TGCCTTTACTCTGCCAGAGTTATATTGCTGGGGTTTTGAAGAAGATCCTATTAAATAAAAGAAT
AAGCAGTATTATTAAGTAGCCCTGCATTTCAGGTTTCCTTGAGTGGCAGGCCAGGCCTGGCCGT
GAACGTTCACTGAAATCATGGCCTCTTGGCCAAGATTGATAGCTTGTGCCTGTCCCTGAGTCCC
AGTCCATCACGAGCAGCTGGTTTCTAAGATGCTATTTCCCGTATAAAGCATGAGACCGTGACTT
GCCAGCCCCACAGAGCCCCGCCCTTGTCCATCACTGGCATCTGGACTCCAGCCTGGGTTGGGGC
AAAGAGGGAAATGAGATCATGTCCTAACCCTGATCCTCTTGTCCCACAGATATCCAGAACCCTG
ACCCT
DNA
Homo sapiens
3′ exon 1 TRAC gene homology flanking sequence, Cas12a target site, 600 bp
SEQ ID NO: 29
GTACCAGCTGAGAGACTCACTATCCAGTGACAAGTCTGTCTGCCTATTCACCGATTTTGATTCT
CAAACAAATGTGTCACAAAGTAAGGATTCTGATGTGTATATCACAGACAAAACTGTGCTAGACA
TGAGGTCTATGGACTTCAAGAGCAACAGTGCTGTGGCCTGGAGCAACAAATCTGACTTTGCATG
TGCAAACGCCTTCAACAACAGCATTATTCCAGAGGACACCTTCTTCCCCAGCCCAGGTAAGGGC
AGCTTTGGTGCCTTCGCAGGCTGTTTCCTTGCTTCAGGAATGGCCAGGTTCTGCCCAGAGCTCT
GGTCAATGATGTCTAAAACTCCTCTGATTGGTGGTCTCGGCCTTATCCATTGCCACCAAAACCC
TCTTTTTACTAAGAAACAGTGAGCCTTGTTCTGGCAGTCCAGAGAATGACACGGGAAAAAAGCA
GATGAAGAGAAGGTGGCAGGAGAGGGCACGTGGCCCAGCCTCAGTCTCTCCAACTGAGTTCCTG
CCTGCCTGCCTTTGCTCAGACTGTTTGCCCCTTACTGCTCTTCTAGGCCTCATTCTAAGCCCCT
TCTCCAAGTTGCCTCTCCTTATTT
DNA
Homo sapiens
Cas12a target site in exon 1 of the TRAC gene
SEQ ID NO: 30
GAGTCTCTCAGCTGGTACACG
DNA
Homo sapiens
PCR synthesized 5′ homology arm, Cas12a target site, 192 nts
SEQ ID NO: 31
ATCACGAGCAGCTGGTTTCTAAGATGCTATTTCCCGTATAAAGCATGAGACCGTGACTTGCCAG
CCCCACAGAGCCCCGCCCTTGTCCATCACTGGCATCTGGACTCCAGCCTGGGTTGGGGCAAAGA
GGGAAATGAGATCATGTCCTAACCCTGATCCTCTTGTCCCACAGATATCCAGAACCCTGACCCT
DNA
Homo sapiens
PCR synthesized 3′ homology arm, Cas12a target site, 159 nts
SEQ ID NO: 32
GTACCAGCTGAGAGACTCACTATCCAGTGACAAGTCTGTCTGCCTATTCACCGATTTTGATTCT
CAAACAAATGTGTCACAAAGTAAGGATTCTGATGTGTATATCACAGACAAAACTGTGCTAGACA
TGAGGTCTATGGACTTCAAGAGCAACAGTGC
DNA
Artificial
Forward primer for generating donor for insertion into Cas12a site
SEQ ID NO: 33
5'-p-ATCACGAGCAGCTGGTTTCT-3′
(p indicates 5’ phosphate)
DNA
Artificial Reverse primer for generating donor for insertion into Cas12a site
SEQ ID NO: 34
5′-mG*mC*mA*CTGTTGCTCTTGAAGTCC-3′
(*indicates phosphorothioate linkage; mA indicates 2′-O-methyladenosine;
mC indicates 2′-O-methylcytidine; mG indicates 2′-O-methylguanosine)
Protein
Homo sapiens
CD8 hinge
SEQ ID NO: 35
AKPTTTPAPRPPTPAPTIASQPLSLRPEACRPAAGGAVHTRGLDFAPR
Protein
Artificial
CD8 hinge + CD28 hinge (long hinge)
SEQ ID NO: 36
AKPTTTPAPRPPTPAPTIASQPLSLRPEACRPAAGGAVHTRGLDFAPRKIEVMYPPPYLDNEKS
NGTIIHVKGKHLCPSPLFPGPSKP
Protein
Homo sapiens
CD8 transmembrane domain
SEQ ID NO: 37
IYIWAPLAGTCGVLLLSLVITLY
Protein
Homo sapiens
4-1BB transmembrane domain
SEQ ID NO: 38
IISFFLALTSTALLFLLFFLTLRFSVV
Protein
Homo sapiens
CD3zeta transmembrane
SEQ ID NO: 39
LCYLLDGILFIYGVILTALFL
Protein
Artificial
CD28 co-stimulatory sequence
SEQ ID NO: 40
RSKRSRLLHSDYMNMTPRRPGPTRKHYQPYAPPRDFAAYRS
Protein
Homo sapiens
OX40 co-stimulatory sequence
SEQ ID NO: 41
ALYLLRRDQRLPPDAHKPPGGGSFRTPIQEEQADAHSTLAKI
Protein
Artificial
CD3zeta, including ITAMs 1, 2, and 3
SEQ ID NO: 42
RVKFSRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
Protein
Artificial
CD3zeta ITAM 1
SEQ ID NO: 43
RVKFSRSADAPAYQQGQNQLYNELNLGRREEYDVLDKR
Protein
Artificial
CD3zeta ITAM 2
SEQ ID NO: 44
RVKFSRSADRGRDPEMGGKPRRKNPQEGLYNELQKDKMAEAYSEIGM
Protein
Artificial
CD3zeta ITAM 3
SEQ ID NO: 45
RVKFSRSADKGERRRGKGHDGLYQGLSTATKDTYDALHMQALPPR
Protein
Artificial
DAR V1 intracellular domain
SEQ ID NO: 46
RSKRSRLLHSDYMNMTPRRPGPTRKHYQPYAPPRDFAAYRSRVKFSRSADAPAYQQGQNQLYNE
LNLGRREEYDVLDKRRGRDPEMGGKPRRKNPQEGLYNELQKDKMAEAYSEIGMKGERRRGKGHD
GLYQGLSTATKDTYDALHMQALPPR
Protein
Artificial
DAR V2a intracellular domain
SEQ ID NO: 47
KRGRKKLLYIFKQPFMRPVQTTQEEDGCSCRFPEEEEGGCELRVKFSRSADAPAYQQGONQLYN
ELNLGRREEYDVLDKRRGRDPEMGGKPRRKNPQEGLYNELQKDKMAEAYSEIGMKGERRRGKGH
DGLYQGLSTATKDTYDALHMQALPPR
Protein
Artificial
DAR V2b and CAR (28Z) intracellular domain
SEQ ID NO: 48
RSKRSRLLHSDYMNMTPRRPGPTRKHYQPYAPPRDFAAYRSRVKFSRSADAPAYQQGQNQLYNE
LNLGRREEYDVLDKRRGRDPEMGGKPRRKNPQEGLYNELQKDKMAEAYSEIGMKGERRRGKGHD
GLYQGLSTATKDTYDALHMQALPPR
Protein
Artificial
DAR V2c intracellular domain
SEQ ID NO: 49
KRGRKKLLYIFKQPFMRPVQTTQEEDGCSCRFPEEEEGGCELRSKRSRLLHSDYMNMTPRRPGP
TRKHYQPYAPPRDFAAYRSRVKFSRSADAPAYQQGQNQLYNELNLGRREEYDVLDKRRGRDPEM
GGKPRRKNPQEGLYNELQKDKMAEAYSEIGMKGERRRGKGHDGLYQGLSTATKDTYDALHMQAL
PPR
Protein
Artificial
DAR V3 and V4 intracellular domain
SEQ ID NO: 50
KRGRKKLLYIFKQPFMRPVQTTQEEDGCSCRFPEEEEGGCELRVKFSRSADKGERRRGKGHDGL
YQGLSTATKDTYDALHMQALPPR
Protein
Artificial
DAR V3 intracellular domain
SEQ ID NO: 51
KRGRKKLLYIFKQPFMRPVQTTQEEDGCSCRFPEEEEGGCELRSKRSRLLHSDYMNMTPRRPGP
TRKHYQPYAPPRDFAAYRSRVKFSRSADKGERRRGKGHDGLYQGLSTATKDTYDAL
Protein
Homo sapiens
CD8a leader sequence
SEQ ID NO: 52
MALPVTALLLPLALLLHAARP
Protein
P2A self-cleaving sequence
SEQ ID NO: 53
GSGATNFSLLKQAGDVEENPGP
Protein
E2A self-cleaving sequence
SEQ ID NO: 54
GSGQCTNYALLKLAGDVESNPGP
Protein
F2A self-cleaving sequence
SEQ ID NO: 55
GSGVKQTLNFDLLKLAGDVESNPGP
Protein
Hinge sequence
SEQ ID NO: 59
EPKSCDKTHT
Protein
Hinge sequence
SEQ ID NO: 56
PAPELLGGP
Protein
CH2 region
SEQ ID NO: 57
SVFLFPPKPKDT
Protein
Hinge sequence
SEQ ID NO: 58
EPKSCDKTHTCPPCPAP ELLGGP

### EXAMPLES

The following examples are meant to be illustrative and can be used to further understand embodiments of the present disclosure and should not be construed as limiting the scope of the present teachings in any way.

### Example 1. Isolation of Human PBMC Cells and Primary T Cells

Primary human T cells were isolated from healthy human donors either from buffy coats (San Diego blood bank), fresh blood, or leukapheresis products (StemCell Technologies Inc., Cambridge, Mass., USA). Peripheral blood mononuclear cells (PBMC) were isolated by density gradient centrifugation. Exemplary methods used for T cell isolation from two donors follow:

Preparation of Donor 1 cells: T cells were isolated from PBMCs by magnetic negative selection using EASYSEP Human T Cell Isolation Kit (StemCell Technologies Inc.) or positive selection and activation by DYNABEADS Human T-Expander CD3/CD28 (Thermo Fisher Scientific, Waltham, Mass., USA) according to manufacturer's instructions. Donor 1 cells were transfected with nucleic acids encoding an CD20 CAR or CD20 DAR to generate transgenic T cells that express the CAR or DAR constructs.

Preparation of Donor 2 cells: To deplete the monocytes, PBMC were plated in a cell culture coated flask for one to two hours. The nonadherent lymphocytes were washed away from the flask and activated with T cell TRANSACT (Miltenyi Biotec, San Diego, Calif., USA) in a new flask according to manufacturer's instructions. Donor 2 cells were transfected with nucleic acids encoding an CD20 CAR or precursor CD20 DAR to generate transgenic T cells that express the CAR or DAR constructs.

### Example 2. Primary T Cell Culture

Primary T cells seeded at density of 10⁶ (1e6) cells per mL were cultured in CTS OPTMIZER T Cell Expansion SFM supplemented with 5% CTS Immune Cell SR (Thermo Fisher Scientific) with 300 U/mL IL-2 (Proleukin). Isolated T cells were either freshly isolated or from frozen stock. Prior to transfection, cells were activated with T Cell TRANSACT (containing CD3 and CD28) (Miltenyi Biotec) at 3 μL/10⁶ (1e6) cells per mL for two to three days. Following transfection with a CAR or DAR construct, T cells were cultured in media with IL-2 at 300 U/mL.

### Example 3. Preparation of CD20 CAR T Cells and CD20 DAR T Cells

Activated T cells (approximately 9×10⁶ (9e6) cells) were transfected with nucleic acids encoding either a CD20 CAR or a CD20 DAR, where the CAR and DAR included the same anti-CD20 antibody heavy and light chain variable domain sequences (SEQ ID NO:3 and SEQ ID NO:11, respectively). The T cells were engineered to knock out the TRAC (T cell receptor alpha constant) gene by insertion of the CAR or DAR construct.

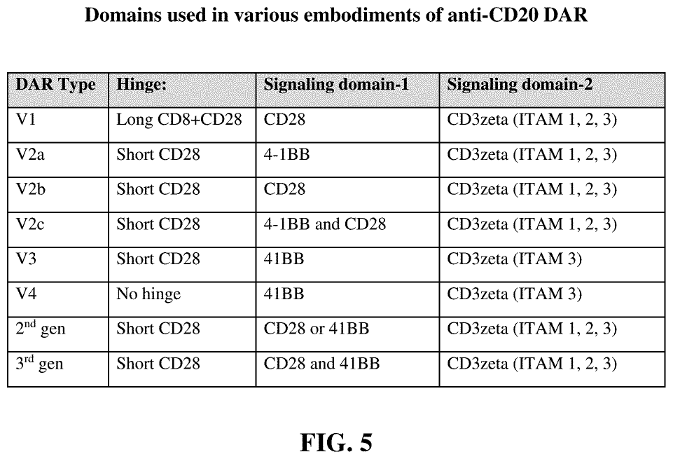

The CD20 DAR construct used to transfect cells was designed for expressing a first polypeptide (SEQ ID NO:14) and a second polypeptide (SEQ ID NO:15) on the cell surface as two polypeptides linked by disulfide bonds. The name designations of various CD20 DAR constructs, with their respective hinge and intracellular regions, is listed in the table provided as FIG. 5. The first polypeptide included, proceeding from the N-terminus to the C-terminus, an anti-CD20 antibody heavy chain variable region sequence provided as SEQ ID NO:3, the anti-CD20 antibody heavy chain constant region (CH1, SEQ ID NO:4), the CD28 hinge region (SEQ ID NO:5), the CD28 transmembrane region (SEQ ID NO:6), the 4-1BB intracellular costimulatory sequence (SEQ ID NO:7), and the CD3ζ intracellular signaling region (ITAM3 only) (SEQ ID NO:8). The precursor first polypeptide encoded by the construct further included at the N terminus the signal peptide of SEQ ID NO:2 to direct the polypeptide to the membrane when synthesized by a cell. This “heavy chain” first polypeptide of the anti-CD20 DAR was designed to be produced along with the “light chain” second polypeptide of the DAR from a single open reading frame by designing a construct in which the sequence encoding the heavy chain polypeptide was fused in frame to a 2A-encoding sequence (encoding the T2A peptide of SEQ ID NO:9) which was in turn fused in-frame to a sequence encoding the second polypeptide of the DAR. The second polypeptide sequence included, proceeding from the N terminus to the C terminus, the anti-CD20 antibody light chain variable region (SEQ ID NO:11) and the anti-CD20 antibody light chain constant region (kappa) (SEQ ID NO:12). The light chain precursor polypeptide encoded by the DAR construct also included at its N terminus a signal peptide for secretion, in this case the signal peptide of SEQ ID NO:10. The continuous open reading frame of the anti-CD20 DAR construct (SEQ ID NO:16) encoded an amino acid sequence that included both polypeptide chains (SEQ ID NO:13) to result in the production of two polypeptides (SEQ ID NO:14 and SEQ ID NO:15), due to the connecting T2A sequence (SEQ ID NO:9) that results in the biosynthesis of the amino acid sequence encoded by the construct as two polypeptides. The two mature polypeptides, the transmembrane engineered first polypeptide (SEQ ID NO:14) and the secreted engineered second polypeptide (SEQ ID NO:15) are assembled via cysteine bridges in their antibody constant domains at the cell exterior, forming a CD20 binding domain.

The CD20 CAR construct (SEQ ID NO:17) encoded a single polypeptide (SEQ ID NO:18) that included the same anti-CD20 antibody heavy chain variable region sequence (SEQ ID NO:3) and the same anti-CD20 antibody light chain variable region sequence (SEQ ID NO:11) as the CD20 DAR. The CD20 CAR construct (SEQ ID NO:17) encoded a CAR having, from the N-terminus to the C-terminus, a signal peptide (SEQ ID NO:2) for secretion, an anti-CD20 antibody light chain variable region (SEQ ID NO:11) followed by a peptide linker (SEQ ID NO:19) connecting the light chain variable region to an anti-CD20 antibody heavy chain variable region (SEQ ID NO:3). The heavy chain variable region (SEQ ID NO:3) was then followed by the CD28 hinge region (SEQ ID NO:5), the CD28 transmembrane region (SEQ ID NO:6), the 4-1BB intracellular costimulatory sequence (SEQ ID NO:7), and the CD3ζ intracellular signaling region (ITAM3 only) (SEQ ID NO:8). Thus, the CD20 CAR, as a single polypeptide, included the same anti-CD20 antibody heavy chain and light chain variable regions, as well as the same CD28 hinge region, CD28 transmembrane region, 4-1BB intracellular costimulatory sequence, and CD3ζ intracellular signaling region, as the CD20 DAR.

The Cas9 RNA-guided endonuclease was used to generate CD20 CAR-T cells and CD20 DAR-T cells with simultaneous knockout of the TRAC gene. For generating CD20 DAR-T cells using Cas9, the CD20 DAR construct described above (SEQ ID NO:16) was cloned downstream of the JeT promoter (SEQ ID NO:20) and the fragment that included the construct plus operably linked promoter was inserted between 5′ and 3′ homology regions of the T cell receptor alpha constant (TRAC) gene (Entrez Gene ID: 28755) (SEQ ID NO:21 and SEQ ID NO:22, respectively) that flanked the target site for Cas9-mediated integration (SEQ ID NO:23) in AAV vector pAAV-MCS. Bacterial clones containing the CD20 DAR construct operably linked to the JeT promoter and flanked by the TRAC gene homology regions were confirmed by sequencing.

Separately, the CD20 CAR construct encoding the CD20 CAR precursor polypeptide (SEQ ID NO:17), was cloned downstream of the JeT promoter (SEQ ID NO:20) and inserted between the same 5′ and 3′ T cell receptor alpha constant (TRAC) gene (Entrez Gene ID: 28755) homology regions (SEQ ID NO:21 and SEQ ID NO:22) flanking the Cas9 target site (SEQ ID NO:23) as used for cloning of the DAR construct, in AAV vector pAAV-MCS. Bacterial clones containing the CD20 CAR construct operably linked to the JeT promoter and flanked by the TRAC gene homology regions were also confirmed by sequencing.

For CAR or DAR knock-in/TCR knockout using Cas9, the RNP complex was made by first combining an Alt-R® CRISPR-Cas9 crRNA that included the target sequence of SEQ ID NO:23 and an Alt-R® CRISPR-Cas9 tracrRNA (both from IDT, Coralville, Iowa) and heating the mixture at 95° C. for 5 min. The mixture was then allowed to cool to room temperature (18-25° C.) on the bench top for approximately 20 min to make a crRNA:tracrRNA duplex. For each transfection, 10 μg wide type SpCas9 protein that included nuclear localization sequences (IDT) was mixed with 200 pmol crRNA:tracrRNA duplex and the mixture was incubated at 4° C. for 30 min to form RNPs.

The donor DNA for Cas9-mediated insertion of the CD20 DAR was generated from a pAAV plasmid that included the CD20 DAR construct of SEQ ID NO:16 flanked by 5′ and 3′ homology sequences (SEQ ID NO:21 and SEQ ID NO:22, respectively) from the TRAC gene. The donor fragment having the shorter homology arms of SEQ ID NO:24 (171 bp) and SEQ ID NO:25 (161 bp) was produced using the forward primer of SEQ ID NO:26 having the sequence: A*TmC*mA*mCGAGCAGCTGGTTTCT, and the reverse primer having the sequence: GACCTCATGTCTAGCACAGTTTTG (SEQ ID NO:27). The forward primer (SEQ ID NO:26) included phosphorothioate bonds between the first and second, third and fourth, and fourth and fifth nucleotides from the 5′ terminus (designated with an asterisk (*)). The nucleotides at the third, fourth, and fifth positions from the 5′-end of the forward oligonucleotide primer were 2′-O-methyl modified (designated as mC, mA, and mC). The reverse primer (SEQ ID NO:27) did not have these modifications but included a 5′-terminal phosphate. PCR was performed essentially as provided above to produce a double-stranded donor DNA molecule having a CD20 DAR construct (SEQ ID NO:16) flanked by homology arms of 171 and 161 bps (SEQ ID NO:24 and SEQ ID NO:25). The resulting double stranded CD20 DAR donor DNA fragment was 2.789 Kilobases in size and was used to transfect activated T cells as a double-stranded molecule together with the Cas9 RNP.

The donor DNA that included the CD20 CAR construct was designed and synthesized in the same way as the donor that included the CD20 DAR construct. The same primers (SEQ ID NO:26 and SEQ ID NO:27) were used to generate the double-stranded donor fragment with 171 and 161 bp homology arms (SEQ ID NO:24 and SEQ ID NO:25).

For transfection of cells with the CD20 CAR or CD20 DAR donor DNA plus a Cas9 RNP targeting exon 1 of the TRAC locus, 3×10⁶ (3e6) cells were mixed with the RNP and 5 μg of the double-stranded donor DNA were added. The cells were electroporated with 540 V, 20 ms pulse width, 1 pulse using Celetrix electroporation equipment (Celetrix) and 20 μL tips. As a control, one T cell population was transfected with the Cas9 RNP but without donor fragment. In the absence of a donor fragment, the RNP will disrupt the targeted gene but no expression construct is inserted. The cells transfected with a targeting RNP but without a donor DNA are therefore referred to as TCR knockout (KO) controls.

The DAR construct was also introduced into cells using the Cas12a RNA-guided endonuclease. To produce donor DNA for insertion via Cas12a, the CD20 DAR construct (SEQ ID NO:16) was cloned downstream of the JeT promoter (SEQ ID NO:20) and inserted between 5′ and 3′ T cell receptor alpha constant (TRAC) gene (Entrez Gene ID: 28755) homology regions (SEQ ID NO:28 and SEQ ID NO:29, respectively) that flanked the target site for Cas12a-mediated integration (SEQ ID NO:30) in AAV vector pAAV-MCS. Bacterial clones containing the CD20 DAR construct operably linked to the JeT promoter and flanked by the TRAC gene homology regions were confirmed by sequencing. The TRAC gene was targeted using a Cas12a ribonucleoprotein complex (RNP) that included a Cas12a crRNA was used that included the target sequence: GAGTCTCTCAGCTGGTA CACG (SEQ ID NO:30) that occurs downstream of a Cas12a PAM (TTTA) in exon 1 of the TRAC gene. The RNP was introduced into the cells together with a donor DNA having 192 bp and 159 bp TRAC gene homology arms (SEQ ID NO:31 and SEQ ID NO:32) flanking the DAR or CAR construct. The donor DNAs were generated from the pAAV plasmid that included the CD20 DAR construct flanked by 645 bp and 600 bp TRAC gene homology sequences (SEQ ID NO:28 and SEQ ID NO:29) by PCR (PrimeSTAR Max Premix (Takara Bio USA)) using a forward primer (ATCACGAGCAGCTGGTTCT; SEQ ID NO:33) that included a 5′ phosphate and a reverse primer (mG*mC*mA*CTGTTGCTCTTGAAGTCC; SEQ ID NO:34) having the 5′-most three bases 2′O methylated and having phosphorothioate bonds linking the first and second, second and third, and third and fourth nucleotides from the 5′ end. The double stranded donor DNA products were purified by AX 500 column (MACHEREY-NAGEL, Dueren, Germany). The eluted DNA fragment was precipitated with isopropanol and the air-dried pellets were resuspended in 50 μL of sterile deionized water.

The Cas12a RNP was formed by incubating 10 μg Cas12a protein that included NLS sequences (A.s. Cas12a, IDT, Coralville, Iowa) and 300 pmoL crRNA that included the Cas12a target sequence (SEQ ID NO:30) (Alt-R® A.s. Cas12a crRNA, IDT) for 15 min at room temperature. Electroporation of the Cas12a RNPs and the double-stranded donor DNA into activated T cells was performed essentially as described above for transfection with Cas9 RNPs. As controls, one T cell population was transformed with the Cas12a RNP but with no donor fragment (knockout (KO) controls).

Following electroporation with either Cas9 or Cas12a RNPs and donor fragments, cells were diluted into culture medium and incubated at 37° C., 5% CO₂ in OpTmizer™ T Cell Expansion SFM supplemented with 5% serum replacement and 300 U/ml IL2 in 37° C. Once cells were in expansion cultures, cell counts were obtained every 2 or 3 days and the cell concentration was maintained at 5×10¹ (5e5) to 1×10⁶ (1e6) per mL.

After ten days in culture, the cells were depleted of CD3-positive cells using CD3 microbeads (MACS Miltenyi Biotec, 130-097-043). The CD3+-depleted cells were analyzed by flow cytometry. Aliquots of 1×105 (1e5) transfected T cells were washed with DPBS/5% human serum albumin, then stained with anti-CD3-BV421 antibody SK7 (BioLegend) and PE or APC conjugated Anti-Rituximab antibody (Acro, RIB-Y35-lmg) for 30-60 minutes at 4° C. CD3 and CD20 DAR or CD20 CAR expression were analyzed using iQue Screener Plus (Intellicyte Co.) or Flow Cytometer Attune NxT (AFC2) (Life Technologies). Negative controls were cells that had been transfected with a Cas9 RNP targeting the first exon of the TRAC gene in the absence of a donor DNA (referred to as TCR knockout cells).

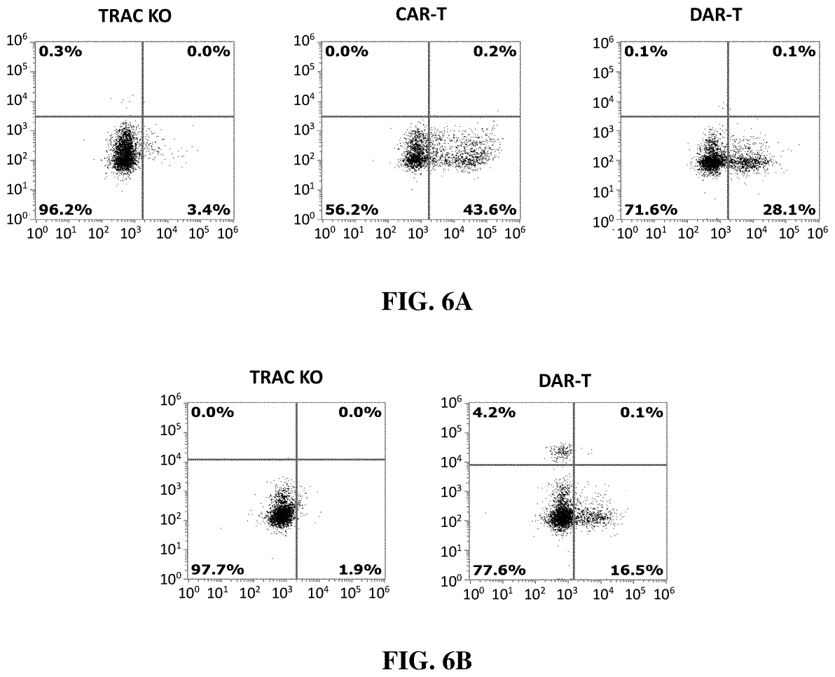

FIG. 6A provides the results of the flow cytometry analysis with cells transfected with a Cas9 RNP. Ten days after transfection and following depletion of CD3-positive cells, no expression of a CD20 construct was detected in cells transfected with the Cas9 RNP targeting the TRAC gene but without a donor fragment including a CAR or DAR construct (leftmost panel, “TRAC KO”). On the other hand, approximately 43.6% of the CD3-negative cells that had been transfected with a CD20 CAR construct donor fragment along with the Cas9 RNP targeting the TRAC locus expressed the CD20 CAR without expressing the TCR (middle panel, “CAR-T”). These TCR knockout/CD20 CAR-expressing cells are referred to in the following examples as CD20 CAR-T cells. Approximately 28.1% of the CD3-negative cells that had been transfected with a CD20 DAR construct donor fragment along with the Cas9 RNP targeting the TRAC locus expressed the CD20 DAR without expressing the TCR (right panel, “DAR-T”). These TCR knockout/CD20 DAR-expressing cells are referred to in the following examples as Cas9 made CD20 DAR-T cells. FIG. 6B provides the results of the flow cytometry analysis with cells transfected with a Cpf1 RNP. Ten days after transfection and following depletion of CD3-positive cells, no expression of a CD20 construct was detected in CD3-negative cells transfected with the Cas12a RNP (for knocking out the TRAC gene) but without a donor fragment (left panel, “TRAC KO”). Meanwhile, 16.5% of the CD3-negative cells that had been transfected with a CD20 DAR construct along with the Cas12a population expressed the CD20 DAR (right panel, “DAR-T”). These TCR knockout/CD20 DAR-expressing cells are also referred to in the following examples as Cpf1 made CD20 DAR-T cells.

### Example 4. Cytotoxicity Assays Using CD20 CAR T Cells and CD20 DAR T Cells

The CD20 CAR-T and CD20 DAR-T cells generated in Example 3 were compared in annexin V-based in vitro cytotoxicity assays using CD20-positive (CD20+) Daudi or CD20-negative (CD20-) K562 cells as targets. The Daudi and K562 cell lines were obtained from ATCC and were transduced using a Firefly Luciferase (FLuc)-F2A-GFP-IRES-Puro Lentivirus (BioSettia GlowCell-16p-l). Single cell clones with luciferase and GFP expression were selected for the Daudi tumor cell line. K562/RFP cells were made similarly by transducing the K562 cells with Firefly Luciferase (FLuc)-T2A-RFP Lentivirus (BioSettia GlowCell-15-1). All tumor cell lines were cultured in RPMI1640 medium (ATCC) supplemented with 10% fetal bovine serum (Sigma).

CD20 CAR-T cells, CD20 DAR-T cells (generated using Cas9 and generated using Cas12a), and, as controls, TCR knockout cells not expressing a CAR or DAR, were subjected to IL2 starvation overnight in complete cell culture medium. The effector T cells were then co-cultured with the GFP or RFP-expressing target tumor cells for 24 hours. In these assays, the number of target tumor cells was fixed at 0.5×10⁶ (5e5)/mL and the number of effector cells varied. The ratio of effector to target cells ranged from 0.06:1 to 5:1 (Daudi targets) or 0.19:1 to 5:1 (K562 targets). After 24 hours of incubation, the cells were incubated with APC-Annexin V (BioLegend) and analyzed by flow cytometry to determine the percentage of dead cells as a percentage of total target cells to determine the specific target cell killing.

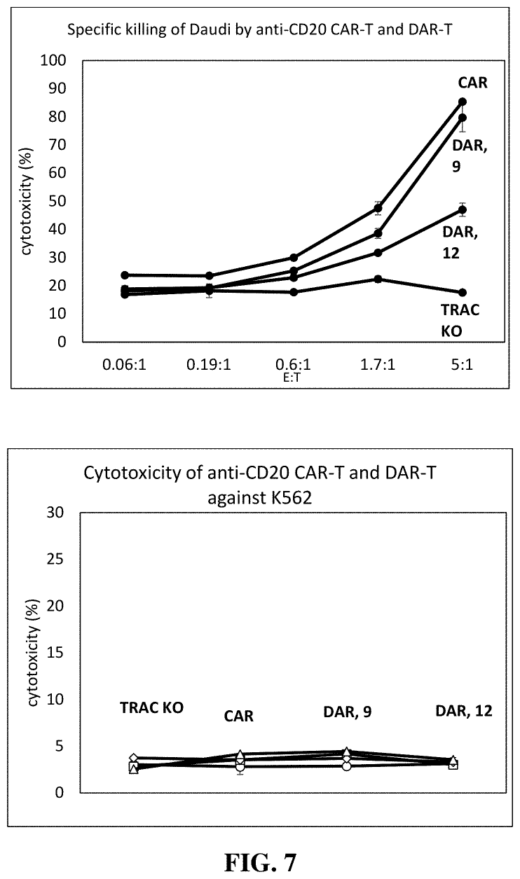

FIG. 7 shows in the upper graph the % cytotoxicity of CD20 CAR-T cell (CAR) and CD20 DAR-T cells made with either Cas9-mediated insertion (DAR 9) or Cas12a-mediated insertion (DAR 12) against CD20-expressing Daudi cells, with TCR knockout T cells (TRAC KO) showing only background cell death. Neither the CD20 CAR-T nor the CD20 DAR-T cells demonstrate enhanced killing of CD20-negative K562 cells (lower graph).

### Example 5. Production of Cytokines by CD20 CAR T Cells and CD20 DAR T Cells

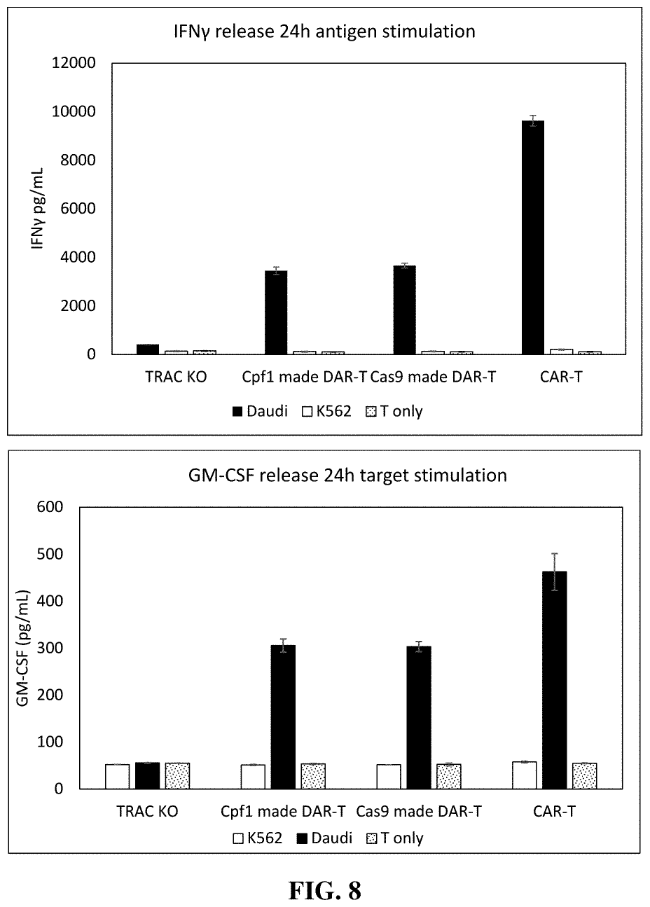

The CD20 DAR-T cells and CD20 CAR-T cells of Example 3 were also tested for cytokine secretion on stimulation with target cells. FIG. 8 shows that both CD20 CAR-T and CD20 DAR-T cells stimulated by co-culturing with CD20-positive Daudi cells for 24 hours produced both interferon gamma (IFNγ, upper graph) and GM-CSF (lower graph), with CD20 CAR-T cells producing a greater amount of both cytokines than either preparation of CD20 DAR-T cells. TCR knockout cells (TRAC KO) were not stimulated to produce either cytokine by Daudi cells, and cytokine production did not rise above background levels (T cells cultured without target cells (“T only”)) when the CD20 CAR-T and CD20 DAR-T cells were co-cultured with CD20-negative K562 cells.

### Example 6. Clonal Expansion of CD20 CAR T Cells and CD20 DAR T Cells

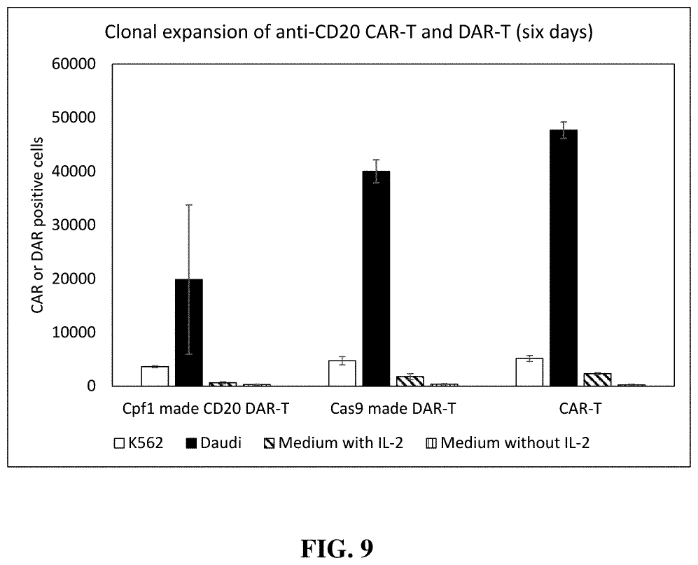

The CD20 DAR-T cells and CD20 CAR-T cells of Example 3 were co-cultured with 10 μg/mL Mitomycin (Sigma, M0440-25MG) pre-treated tumor cells expressing CD20, and as a control, CD20-negative cells, for 6 days in the complete cell culture medium to determine the degree to which the CAR and DAR T cells were stimulated to divide by target cells. At the end of the six day culture period, cells were analyzed by flow cytometry for expression of the CAR or DAR essentially as described in Example 3. FIG. 9 shows that both CD20 CAR-T and CD20 DAR-T cells greatly increased in number after co-culturing with CD20-positive Daudi cells, but there was minimal expansion of both CAR and DAR T cells on CD20-negative K562 cells.

### Example 7. In Vivo Study Using CD20 DAR-T Cells and CD20 CAR-T Cells

Tumoricidal activity of transgenic T cells expressing either the anti-CD20 DAR made using either Cas9 or Cas12a insertion of the DAR construct or anti-CD20 CAR was tested in a Daudi-Fluc (Daudi cells expressing firefly luciferase) xenograft mouse model. Eight week old female NSG mice injected intravenously into the tail with a total 5×10⁵ (5e5) Daudi-Fluc cells suspended in 200 μL PBS. The animals selected in study were randomized into different groups and three days later were treated with TRAC knockout T cells (3×10⁷ cells), CD20 CAR-T cells, CD20 DAR-T cells produced using Cas9, or CD20 DAR-T cells produced using Cpf1 in 200 μL PBS, using ten mice per treatment group. The dose of CAR-T and DAR-T cells was 6×10⁶ CAR or DAR positive cells, where the total number of cells injected was adjusted by the percentage of CAR or DAR positive cells in the population to provide an equal number of CAR or DAR positive cells in the dose. PBS alone (200 μL) was also injected as a further control. Knockout, CAR, and DAR T cells used in the experiment were produced from cells of the same single donor. The single treatment of either PBS, control TCR-knocked-out T cells, transgenic T cells expressing anti-CD20 DAR or transgenic T cells expressing anti-CD20 CAR was administered via the tail. Tumor growth was monitored weekly by measuring total photon flux with an IVIS Lumina III In Vivo Imaging System (Perkin Elmer Health Sciences, Inc) on the dorsal side of each mouse weekly after tumor cell inoculation. Blood samples were collected from each animal at day 1 after administration of the T cell dose and weekly thereafter. The blood samples were analyzed via flow cytometry for percent and total number of CD45-positive cells, anti-CD20 DAR cells, anti-CD20 CAR cells, and CD3-negative cells.

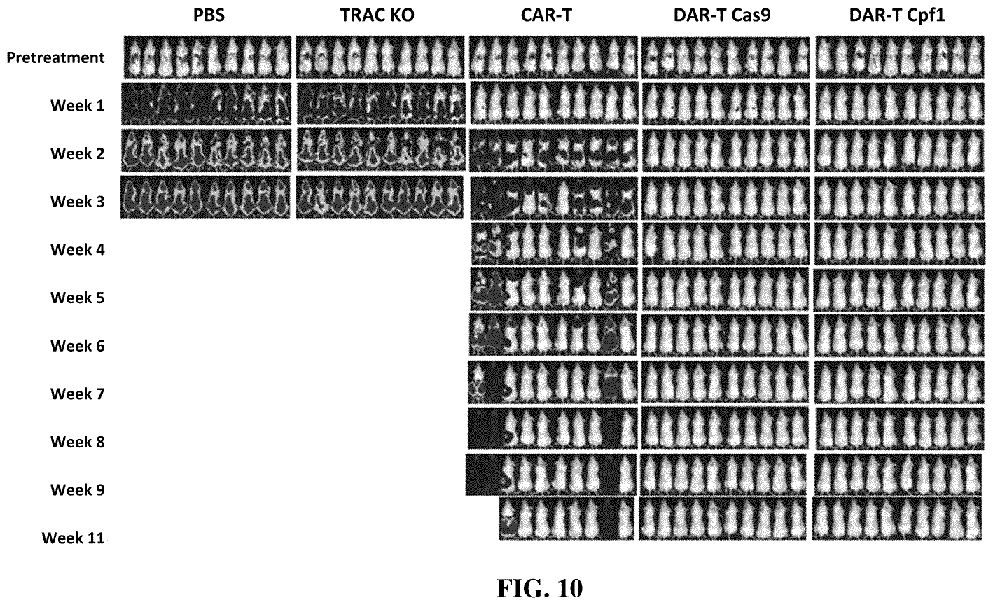

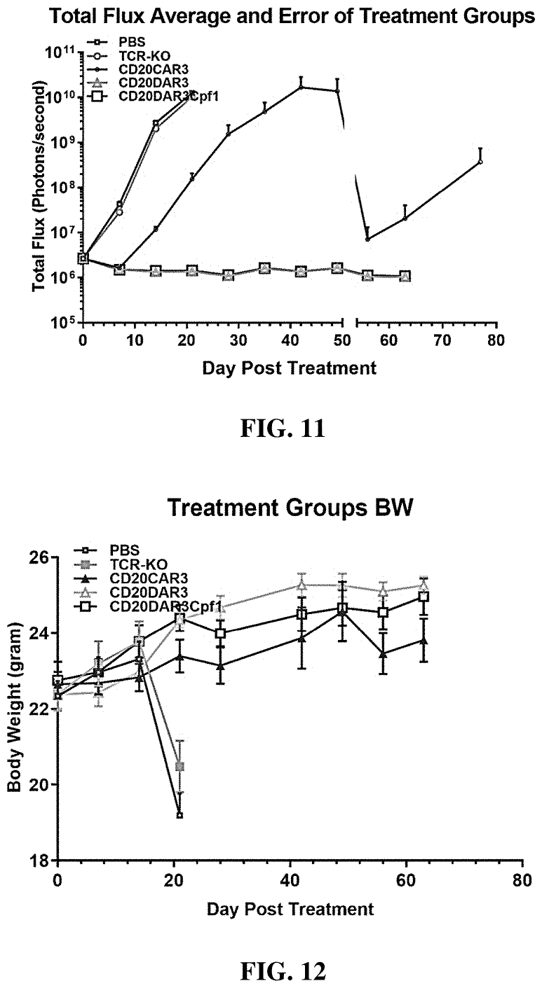

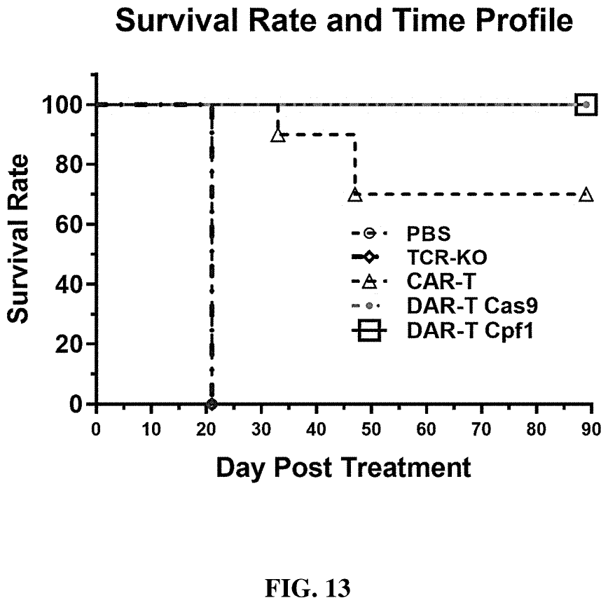

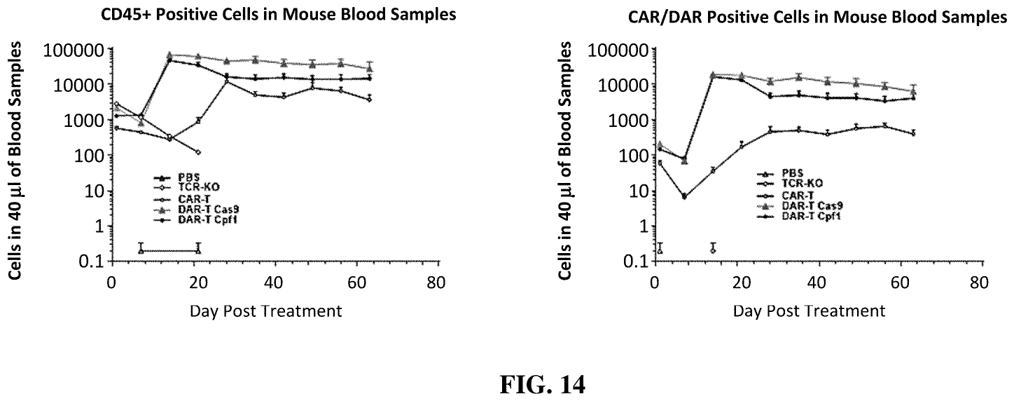

FIG. 10 provides the in vivo imaging of the mice over the course of the study, demonstrating the dramatically improved results of DAR-T cells, regardless of the nuclease used for construct insertion, when compared with CAR-T cells. Treatment with either Cas9-produced or Cas12a-produced CD20 DAR-T cells was highly effective, essentially eradicating tumor and preventing any tumor recurrence in all mice receiving these treatments by week 11. The CAR-T treatment group on the other hand experienced tumor progression or recurrence in at least some of the mice until the end of the study at week 11, with three of the ten members of the group succumbing to tumor by that endpoint. FIG. 11 shows the average increase in total flux over time for each of the treatment groups, again demonstrating the effectiveness of the CD20 DAR-T cells at eliminating tumor cells, and the improved effects of CD20 DAR-T cells over CD20 CAR-T cells in eradicating tumor. Body weights of the mice over the course of the experiment is provided in FIG. 12, where no obvious weight loss was observed in the treatment groups. The survival curves provided in FIG. 13 demonstrate that all tumor-inoculated mice treated with CD20 DAR-T cells survived the eleven-week study, compared with 70% survivorship for the group treated with CD20 CAR-T cells.

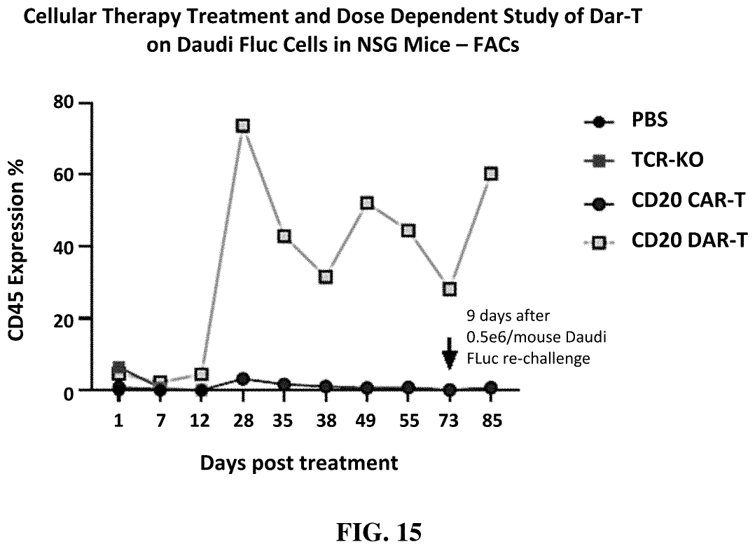

Peripheral blood sampled from mice treated with TCR knockout T cells, CD20 CAR-T cells, and CD20 DAR-T cells was analyzed for the presence of introduced T cells using an antibody that recognized human CD45 and for CD20 CAR or DAR positive cells by flow cytometry. The results are shown in FIG. 14. Human T cells were found in the mice treated with both CD20 CAR-T cells and CD20 DAR-T cells throughout the 9 weeks tested after the initial infusion of the cells (left panel). The right panel of FIG. 14 shows that cells expressing CAR or DAR constructs were found in the peripheral blood of mice were also detected nine weeks after treatment with cells expressing these constructs, with a much higher number of DAR-positive cells found in the DAR-treated mice than the number of CAR-positive cells found in the CAR-treated mice.

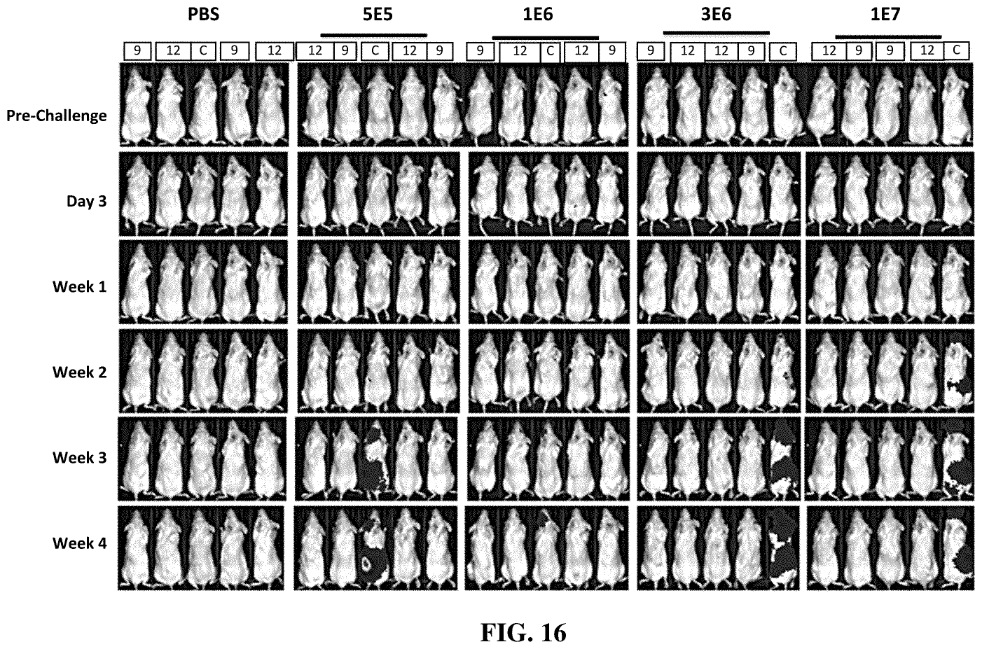

A pilot experiment was performed in which mice (three mice per group) that had been inoculated with tumor cells and then treated with 6×10⁶ CD20 CAR-T cells, CD20 DAR-T cells, or TRAC knockout cells as outlined above were re-challenged with a second inoculation of tumor cells. The peripheral blood of the mice was analyzed before and after T cell treatment and re-challenge with the second tumor inoculum with an antibody recognizing human CD45, a marker present on lymphocytes, including T cells. FIG. 15 shows the percent human CD45 expression in isolated cells from the treatment groups. The graph shows that there was very little increase in CD45+ cells after introduction of CAR-T cells, peaking at about day 28 post-treatment and then declining to undetectable levels without expanding after re-challenge. TRAC knockout T cell and PBS treatment controls showed no CD45+ expression increase over the course of the experiment. DAR-treated mice however showed a higher level of human T cells throughout the study as compared to CAR-treated mice, declining over time but increasing again after re-challenge with tumor cells, indicating that persistent DAR-T cells were able to expand when stimulated by new tumor.

### Example 8. In Vivo Re-Challenge Study

A tumor rechallenge study was performed using mice from the in vivo study detailed above in Example 7. The mice were re-challenged with tumor approximately 13 weeks after the T cell dosing and approximately thirteen and one half weeks after the original tumor inoculation. Tumor-free mice that had been treated with CD20 CAR-T cells or DAR-T cells produced using either Cas9 or Cas12a were randomized into 5 groups of five mice each, each of which included 1 mouse previously treated with CD20 CAR-T cells (“C”), 2 mice treated with CD20 DAR-T cells made with Cas9 (“9”), and 2 mice treated with CD20 DAR-T cells made with Cas12a (“12”). One group received PBS only, one group received 5×10⁵ (5e5) Daudi-Fluc cells, a third group received 10⁶ (1e6) Daudi-Fluc cells, a fourth group received 3×10⁶ (3e6) Daudi-Fluc cells, and a fourth group received 10⁷ (1e7) Daudi-Fluc cells injected into the tail vein.

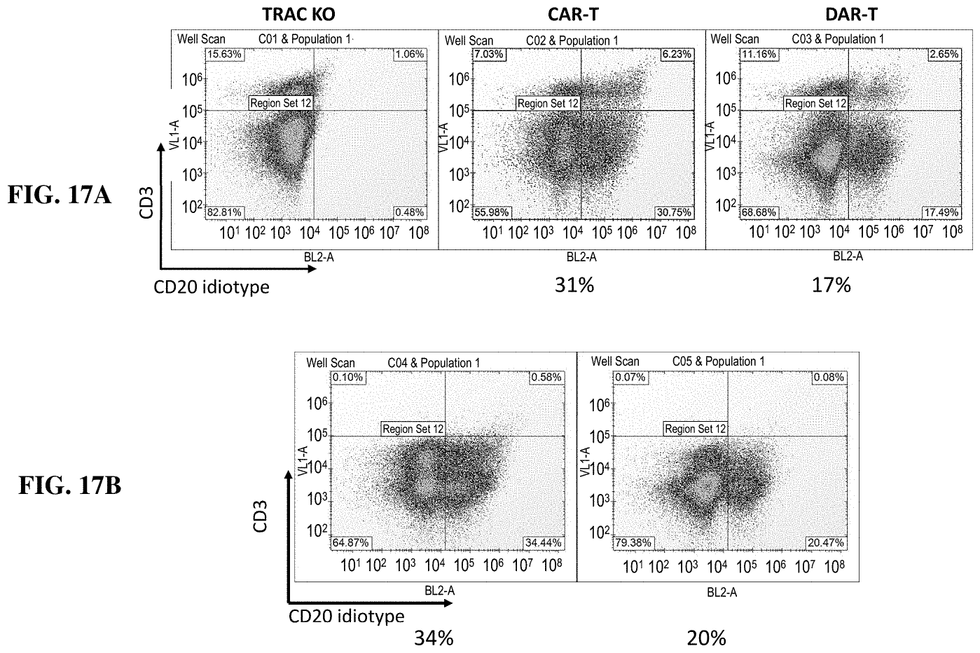

FIG. 16 shows the in vivo imaging of the mice over the next four weeks following the tumor rechallenge inoculations. The mouse in the left of each five mouse panel for inoculation with PBS (control), 5×10¹ (5e5) Daudi-Fluc cells, and 10⁶ (1e6) Daudi-Fluc cells is the mouse previously treated with CAR-T cells (denoted “C”), while the other four mice of the panels were treated with DAR-T cells (denoted “9” or “12”). The right-most mouse in the five mouse panel for treatment with 3×10⁶ (3e6) Daudi-Fluc cells and 10⁷ (1e7) Daudi-Fluc cells is the mouse previously treated with CAR-T cells, while the other four mice of those panels were treated with DAR-T cells. In each group where the mice were re-challenged with tumor cells, tumor was only re-established in mice that received CAR-T cell treatment. None of the mice that had previously received DAR-T cell treatments were observed to develop tumors after rechallenge more than 13 weeks after the DAR-T cell treatment, while each of the mice that had received CD20 CAR-T treatment developed tumor by the end of the four week post-challenge period.

### Example 9. Preparation of CD20 CAR T Cells and CD20 DAR T Cells

CD20 DAR-T cells and CD20 CAR-T cells were produced for use in a dosing study. The DAR and CAR constructs described in Example 3 were used to generate donor fragments with homology arms flanking a Cas9 target site and were independently introduced into the genome of primary human T cells with simultaneous knockout of the TRAC gene using CRISPR/Cas technology with the Cas9 RNA-guided endonuclease essentially as described in Example 3.

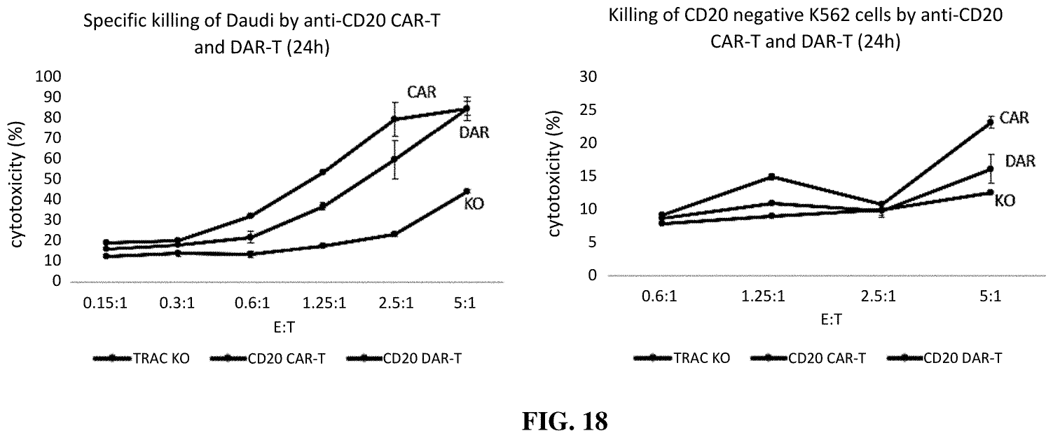

After expanding the cells in culture, depletion of CD3+ cells was performed as described in Example 3 and the cell populations were analyzed by flow cytometry. FIG. 17A shows that eleven days after transfection and prior to depletion of CD3-positive cells, approximately 31% of CAR construct-transfected cells expressed the CD20 construct in the absence of CD3 expression, and approximately 17% of DAR construct-transfected cells expressed the CD20 construct in the absence of CD3 expression. Approximately 13% of the cells of the CD20 CAR-transfected culture as a whole and approximately 14% of the cells of the CD20 DAR-transfected culture as a whole were CD3+(expressed the T cell receptor) prior to CD3+ cell depletion. FIG. 17B shows that following depletion of CD3-positive cells, approximately 34% of CAR construct-transfected cells expressed the CD20 construct in the absence of CD3 expression, and approximately 20% of DAR construct-transfected cells expressed the CD20 construct in the absence of CD3 expression. CD3+ cells were substantially absent from these cultures, so that essentially all of the cells of the CD3-positive cell depleted cultures expressing a CAR or DAR construct also did not express the T cell receptor.

### Example 10. Cytotoxicity Assays Using CD20 CAR T Cells and CD20 DAR T Cells

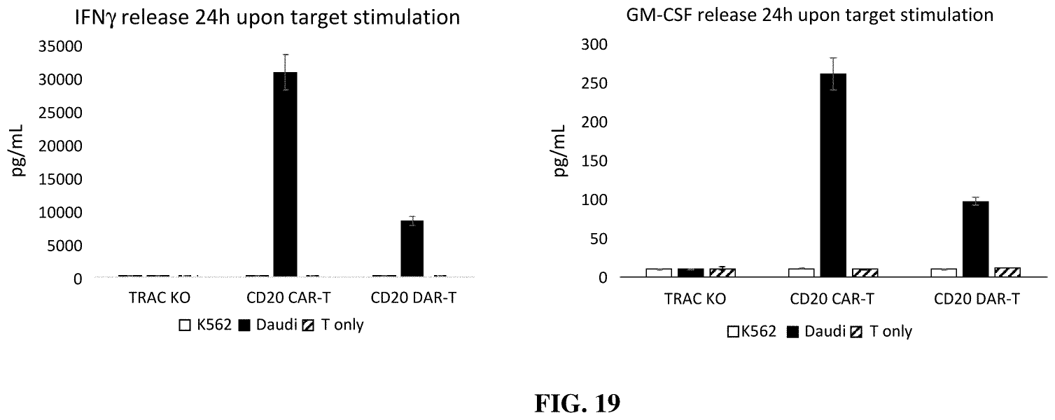

The CD3+-depleted CD20 CAR-T and CD20 DAR-T cells of Example 9 were compared in annexin V-based in vitro cytotoxicity assays using CD20-positive (CD20+) Daudi or CD20-negative (CD20-) K562 cells as targets as described in Example 4. In these assays, the number of target tumor cells was fixed at 0.5×10⁶/mL and the number of effector cells varied. The ratio of effector to target cells ranged from 0.15:1 to 5:1 (Daudi targets) or 0.6:1 to 5:1 (K562 targets). FIG. 18 shows that both CD20 CAR-T and CD20 DAR-T cells exhibit CD20-specific cytotoxicity toward Daudi cells. The % cytotoxicity of CD20 DAR-T cells against CD20-expressing Daudi cells is somewhat lower than that of CD20 CAR-T cells except at the highest effector:target ratio.

### Example 11. Production of Cytokines by CD20 CAR T Cells and CD20 DAR T Cells

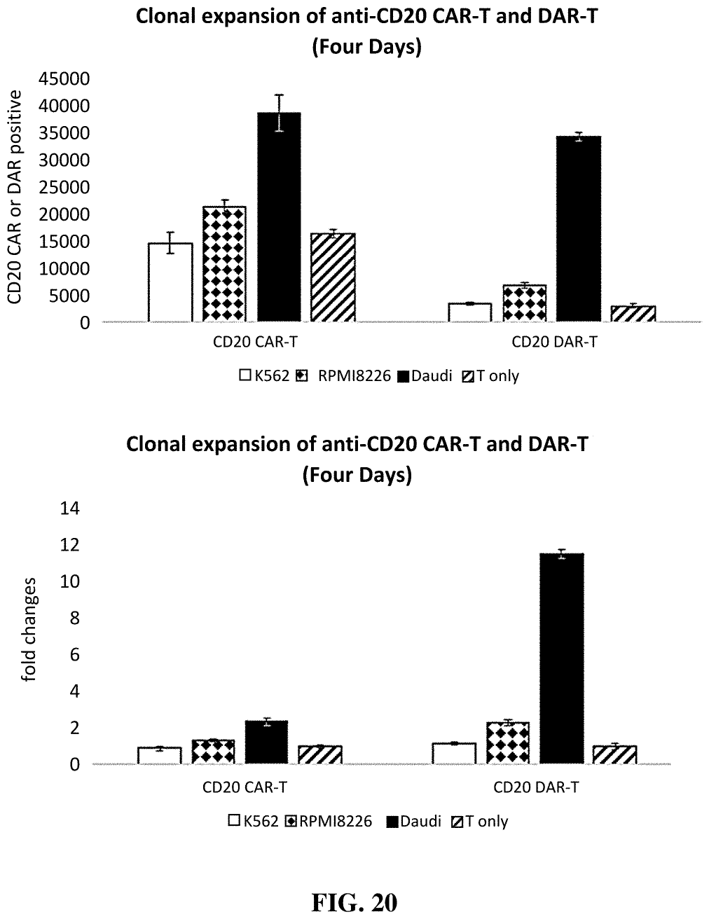

The CD3+-depleted CD20 DAR-T cells and CD20 CAR-T cells of Example 9 were also tested for cytokine secretion on stimulation with target cells. FIG. 19 shows that both CD20 CAR-T and CD20 DAR-T cells stimulated by co-culturing with CD20-positive Daudi cells for 24 hours produced both interferon gamma (IFNγ, left graph) and GM-CSF (right graph), with CD20 CAR-T cells producing a greater amount of both cytokines than CD20 DAR-T cells. Cytokine production did not rise above background levels when the CD20 CAR-T and CD20 DAR-T cells were co-cultured with CD20-negative K562 cells.

### Example 12. Clonal Expansion of CD20 CAR T Cells and CD20 DAR T Cells

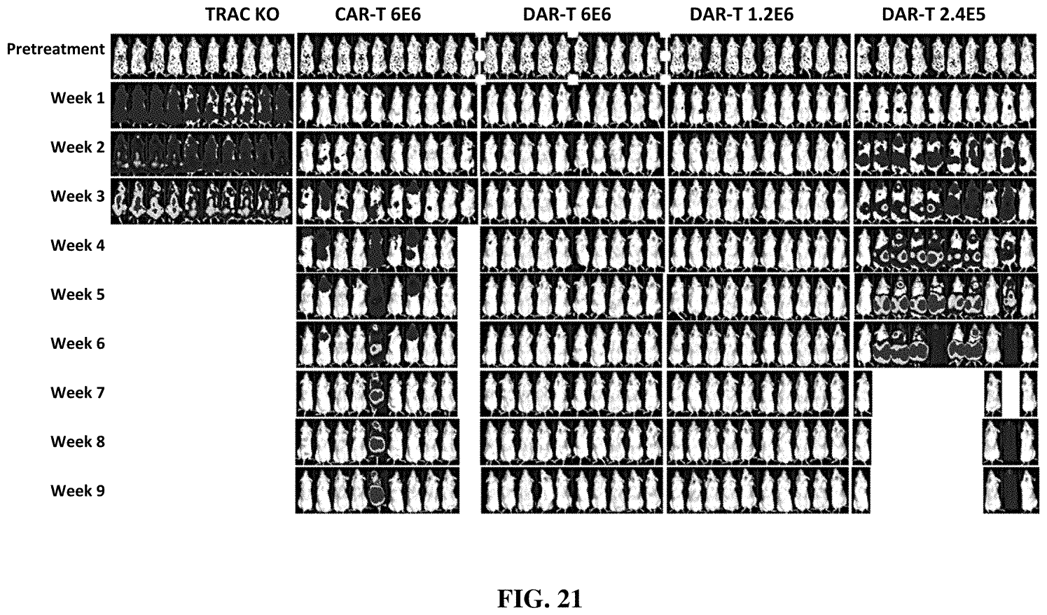

The CD3+-depleted CD20 DAR-T cells and CD20 CAR-T cells of Example 9 were co-cultured with 10 μg/mL Mitomycin (Sigma, M0440-25MG) pre-treated tumor cells expressing CD20, and as a control, CD20-negative cells, for 4 days in the cell culture medium with IL-2 to determine the degree to which the CAR and DAR T cells were stimulated to divide by target cells. At the end of the 4-day culture period, cells were analyzed by flow cytometry for expression of the CAR or DAR essentially as described in Example 3. FIG. 20 (left graph) shows that both CD20 CAR-T and CD20 DAR-T cells greatly increased in number stimulated by co-culturing with CD20-positive Daudi cells. The effect was highly specific for co-culture with CD20-expressing cells for CD20 DAR-T cells, whereas CD20 CAR-T cells showed some expansion on CD20-negative cells. This difference between CAR-T and DAR-T response to CD20-negative and CD-positive cells can be seen in the fold-change (right graph of FIG. 20) of expansion, where CD20 DAR-T culture expansion on CD20+ cells was greater than ten-fold over four days while CD20 CAR-T culture expansion on CD20+ cells was less than 2.5-fold over the same time frame.

### Example 13. In Vivo Dosage Study of CD20 DAR-T Cells

The transgenic T cells produced in Example 9 expressing anti-CD20 CAR anti-CD20 DAR and depleted of CD3+ cells were used in a dosage study in the Daudi-Fluc xenograft mouse model. A total 0.5×10⁶ (5e5) Daudi-Fluc cells were suspended in 200 μL PBS, and then injected intravenously into the tail veins of eight week old female NSG mice. The animals selected in study were randomized into different groups and three days later were treated with TRAC knockout T cells (4.4×10⁷ cells, 89% viability), CD20 CAR-T cells (6×10⁶ cells), or CD20 DAR-T cells at three different dosages (2.4×10⁵, 1.2×10⁶, and 6×10⁶ cells; the number of cells was adjusted by the percentage of CAR or DAR positive cells in the population to the appropriate number of CAR or DAR positive cells in the dose) in 200 μL PBS, using ten mice per treatment group. Knockout, CAR, and DAR T cells used in the experiment were from the same single donor. The single treatment of either PBS, control TCR-knocked-out T cells, transgenic T cells expressing anti-CD20 DAR or transgenic T cells expressing anti-CD20 CAR was administered via the tail. Tumor growth was monitored weekly by measuring total photon flux with an IVIS Lumina III In Vivo Imaging System (Perkin Elmer Health Sciences, Inc) on the dorsal side of each mouse weekly after tumor cell inoculation. Blood samples were collected from each animal at day 1 after administration of the T cell dose and weekly thereafter. The blood samples were analyzed via flow cytometry for percent and total number of CD45-positive cells, anti-CD20 DAR cells, anti-CD20 CAR cells, and CD3-negative cells.

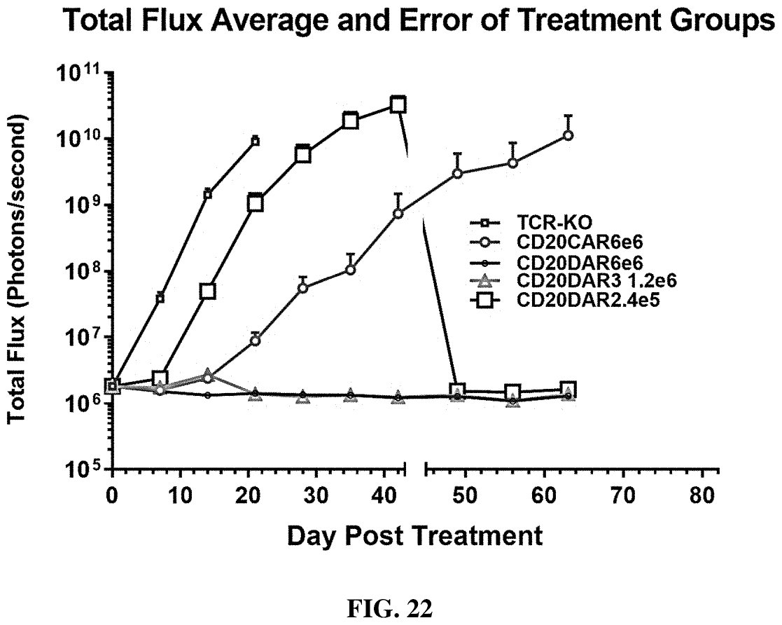

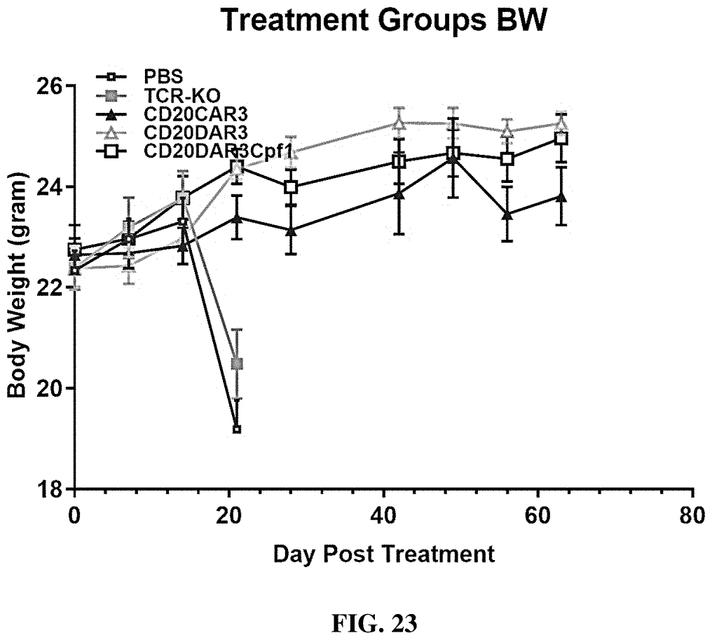

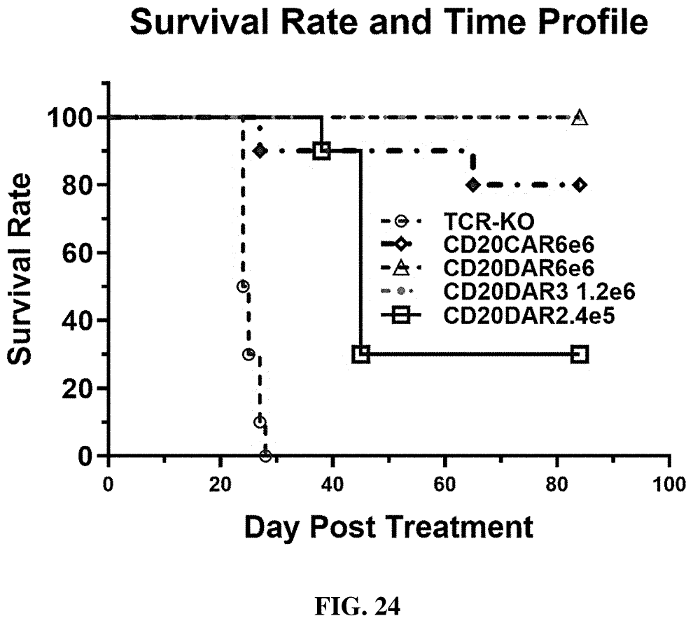

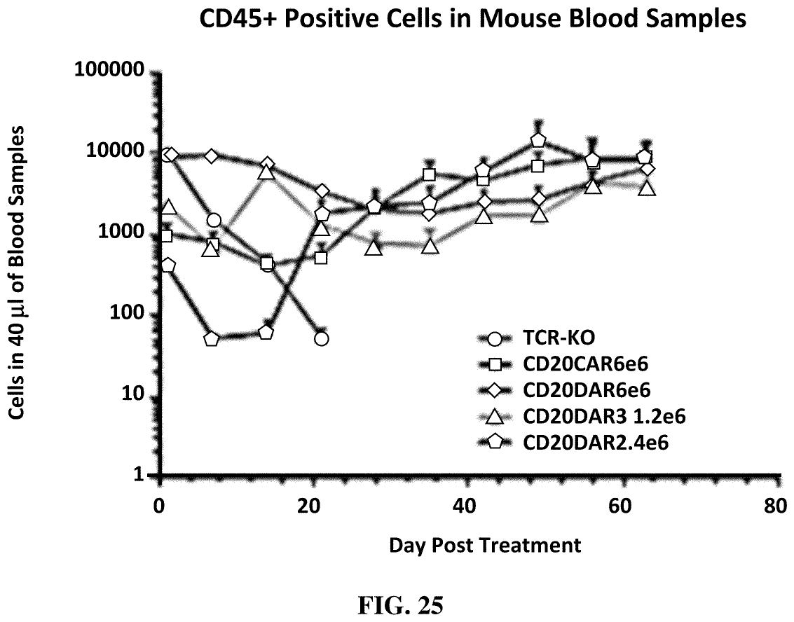

FIG. 21 provides the in vivo imaging of the mice over the course of the study, demonstrating improved results of CD20 DAR-T cells at dosages of 1.2×10⁶ (1.2e6) and 6×10⁶ (6e6) cells when compared with CD20 CAR-T cells dosed at 6×10⁶ (6e6) cells. Treatment with either 1.2×10⁶ or 6×10⁶ CD20 DAR-T cells was highly effective, essentially eradicating tumor in mice receiving these treatments by week 9 without recurrence, whereas tumor developed in mice treated with CD20 CAR-T cells dosed at 6×10⁶ cells and not all mice of the CAR-T treatment group were free of tumor by week 9. FIG. 22 shows the average increase in total flux over time for each of the treatment groups, again demonstrating the effectiveness of the CD20 DAR-T cells at reducing tumor, and the improved effects of CD20 DAR-T cells at both intermediate and high doses over CD20 CAR-T cells given at high dose. Body weights of the mice over the course of the experiment is provided in FIG. 23, where no obvious weight loss was observed in the treatment groups. The survival curves are provided in FIG. 24 and demonstrate that all tumor-inoculated mice treated with either 1.2×10⁶ or 6×10⁶ CD20 DAR-T cells survived the nine-week study, compared with 80% survivorship for the group treated with 6×10⁶ CD20 CAR-T cells.

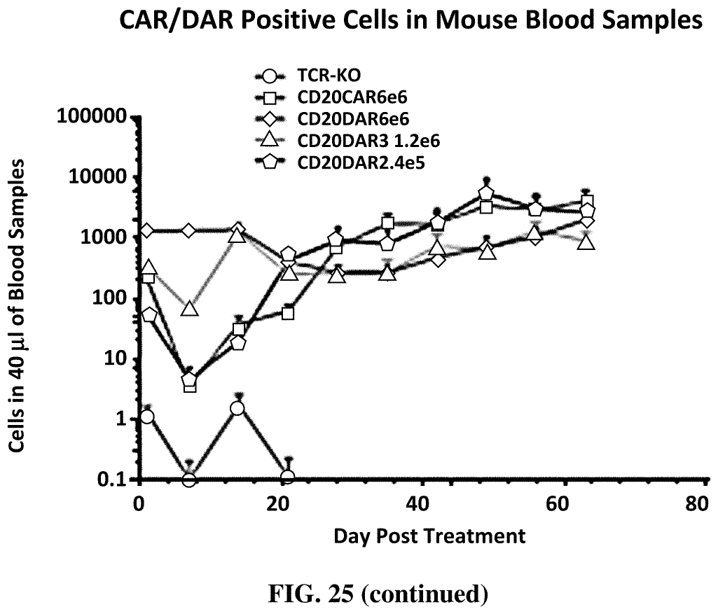

Peripheral blood sampled from mice treated with TCR knockout T cells, CD20 CAR-T cells, and CD20 DAR-T cells was analyzed for the presence of introduced T cells using an antibody that recognized human CD45 and for CD20 CAR or DAR positive cells by flow cytometry. The results are shown in FIG. 25. Human T cells were found in the mice treated with both CD20 CAR-T cells and CD20 DAR-T cells throughout the 9 weeks after the initial infusion of the cells (first panel). The second panel of FIG. 25 shows that cells expressing CAR or DAR constructs were also detected in the peripheral blood of mice nine weeks after treatment with cells expressing these constructs.

1. A genetically modified host cell, or a population of genetically modified host cells, expressing a dimeric antigen receptor (DAR) that binds CD20, wherein the DAR comprises:
(I)
a. a first polypeptide chain comprising a plurality of polypeptide regions ordered from the amino terminus to the carboxyl terminus: (i) an antibody heavy chain variable region, (ii) an antibody heavy chain constant region, (iii) a transmembrane region, and (iv) an intracellular region; and
b. a second polypeptide chain comprising a plurality of polypeptide regions ordered from the amino terminus to the carboxyl terminus: (i) an antibody light chain variable region and (ii) an antibody light chain constant region;
wherein the antibody heavy chain constant region and the antibody light chain constant region form a dimerization domain for formation of the DAR, and wherein the antibody heavy chain variable region and the antibody light chain variable region form an antigen binding domain that binds CD20; or
(II)
a. a first polypeptide chain comprising a plurality of polypeptide regions ordered from the amino terminus to the carboxyl terminus: (i) an antibody light chain variable region, (ii) an antibody light chain constant region, (iii) a transmembrane region, and (iv) an intracellular region; and
b. a second polypeptide chain comprising a plurality of polypeptide regions ordered from the amino terminus to the carboxyl terminus: (i) an antibody heavy chain variable region and (ii) an antibody heavy chain constant region;
wherein the antibody heavy chain constant region and the antibody light chain constant region form a dimerization domain for formation of the DAR, and wherein the antibody heavy chain variable region and the antibody light chain variable region form an antigen binding domain that binds CD20.

2. (canceled)

3. The genetically modified host cell, or the population of genetically modified host cells, according to claim 1, wherein the antibody heavy chain constant region and the antibody light chain constant region dimerize via one or two disulfide bonds.

4. (canceled)

5. The genetically modified host cell, or a population of genetically modified host cells, according to claim 1, further comprising a hinge region in part a), where the hinge region is between the antibody constant region and the transmembrane region, optionally wherein the hinge region comprises a hinge sequence from an antibody selected from a group consisting of IgG, IgA, IgM, IgE and IgD, the hinge region comprises a CD28 hinge region, or the hinge region comprises a CPPC (SEQ ID NO: 61) or SPPC (SEQ ID NO: 62) amino acid sequence.

6-8. (canceled)

9. The genetically modified host cell, or the population of genetically modified host cells, according to claim 1, wherein the transmembrane region comprises a transmembrane sequence from CD28, or wherein the intracellular region comprises an intracellular amino acid sequence comprising a 4-1BB intracellular region (SEQ ID NO:7) and CD3zeta having ITAM 3 (SEQ ID NO:8), or an intracellular amino acid sequence having at least 95% identity thereto.

10. (canceled)

11. The genetically modified host cell, or the population of genetically modified host cells, according to claim 1, wherein the antibody heavy chain variable region comprises an amino acid sequence having at least 95% identity to SEQ ID NO:3, and
optionally wherein the antibody heavy chain constant region comprises an amino acid sequence having at least 95% identity to SEQ ID NO:4, the antibody light chain variable region comprises an amino acid sequence having at least 95% identity to SEQ ID NO:11, or the antibody light chain constant region comprises an amino acid sequence having at least 95% identity to SEQ ID NO:12.

12-14. (canceled)

15. The genetically modified host cell, or the population of genetically modified host cells, according to claim 3, wherein the hinge region comprises an amino acid sequence having at least 95% identity to SEQ ID NO:5.

16. The genetically modified host cell, or the population of genetically modified host cells, according to claim 1, wherein the transmembrane region comprises an amino acid sequence having at least 95% identity to SEQ ID NO:6.

17. The genetically modified host cell, or the population of genetically modified host cells, according to claim 1, wherein the intracellular region comprises a combination of:
i) a 4-1BB intracellular costimulatory sequence (SEQ ID NO:7); and
ii) a CD3zeta amino acid sequence comprising ITAM 3 (SEQ ID NO:8).

18. (canceled)

19. The genetically modified host cell, or the population of genetically modified host cells, according to claim 1, wherein in (I) the first polypeptide chain comprises the amino acid sequence of SEQ ID NO:14, and
optionally wherein in (I), the second polypeptide chain comprises the amino acid sequence of SEQ ID NO:15.

20. (canceled)

21. (canceled)

22-24. (canceled)

25. The genetically modified host cell, or the population of genetically modified host cells, according to claim 1, wherein
a) the first polypeptide chain comprises a plurality of polypeptide regions ordered from the amino terminus to the carboxyl terminus: (i) a CD20 antibody heavy chain variable region comprising an amino acid sequence having at least 95% identity to SEQ ID NO:3; (ii) a CD20 antibody heavy chain constant region comprising an amino acid sequence having at least 95% identity to SEQ ID NO:4; (iii) a hinge region comprising a CD28 hinge region comprising an amino acid sequence having at least 95% identity to SEQ ID NO:5; (iv) a CD28 transmembrane region comprising an amino acid sequence having at least 95% identity to SEQ ID NO:6; and (v) an intracellular region comprising a 4-1BB and a CD3zeta ITAM 3, intracellular sequences comprising the amino acid sequence of SEQ ID NOS:7 and 8, respectively; and wherein
b) the second polypeptide chain comprising a plurality of polypeptide regions ordered from the amino terminus to the carboxyl terminus: (i) a CD20 antibody light chain variable region comprising an amino acid sequence having at least 95% identity to SEQ ID NO:11; and (ii) a CD20 antibody light chain constant region comprising an amino acid sequence having at least 95% identity to SEQ ID NO:12,
wherein the antibody heavy chain constant region and the antibody light chain constant region form a dimerization domain, and
wherein the antibody heavy chain variable region and the antibody light chain variable region form an antigen binding domain that binds a CD20 protein.

26. (canceled)

27. The genetically modified host cell, or the population of genetically modified host cells, according to claim 21, wherein in (I) (a) the first polypeptide chain comprises (i) a CD20 antibody heavy chain variable region comprising the amino acid sequence of SEQ ID NO:3; and b) the second polypeptide chain comprises (i) a CD20 antibody light chain variable region comprising the amino acid sequence of SEQ ID NO:11.

28. The genetically modified host cell, or the population of genetically modified host cells, according to claim 1, wherein the genetically modified host cell or population of genetically modified host cells comprises a nucleic acid sequence encoding the first polypeptide chain and a nucleic acid sequence encoding the second polypeptide chain of the DAR.

29. The genetically modified host cell, or the population of genetically modified host cells, according to claim 28, wherein the population comprises a sequence encoding the polypeptide of SEQ ID NO:14 and the polypeptide of SEQ ID NO:15.

30. The genetically modified host cell or the population of genetically modified host cells, according to claim 28, wherein nucleic acid sequence encoding the first polypeptide and a nucleic acid sequence encoding the second polypeptide are part of a single continuous open reading frame, wherein the open reading frame includes a sequence encoding a peptide that allows production of the first and second polypeptides from the open reading frame, and
optionally wherein the peptide is a T2A, P2A, E2A, or F2A sequence.

31. (canceled)

32. The genetically modified host cell, or the population of genetically modified host cells, according to claim 1, comprising T lymphocytes, NK (natural killer) cells, macrophages, dendritic cells, mast cells, eosinophils, B lymphocytes or monocytes, and
optionally wherein the population of genetically modified host cells are primary cells, the population of genetically modified host cells are human cells, or the population of genetically modified host cells comprise T cells.

33-35. (canceled)

36. The population of host cells according to claim 32, wherein the population of genetically modified host cells comprise T cells and less than 1% of the population expresses the T cell receptor and greater than 20% of the population expresses the DAR.

37. The population of genetically modified cells of claim 36, wherein the T cells are primary human T cells.

38. A pharmaceutical composition comprising a pharmaceutically-acceptable excipient and the population of genetically modified host cells of claim 37.

39. A pharmaceutical composition comprising a pharmaceutically-acceptable excipient and the population of genetically modified host cells of claim 21.

40. A pharmaceutical composition comprising a pharmaceutically-acceptable excipient and the population of genetically modified host cells of claim 29.

41. At least one nucleic acid molecule encoding:
(I)
a) a first polypeptide chain comprising a plurality of polypeptide regions ordered from the amino terminus to the carboxyl terminus: (i) an antibody heavy chain variable region, (ii) an antibody heavy chain constant region, (iii) a transmembrane region, and (iv) an intracellular region; and
b) a second polypeptide chain comprising a plurality of polypeptide regions ordered from the amino terminus to the carboxyl terminus: (i) an antibody light chain variable region and (ii) an antibody light chain constant region;
wherein the antibody heavy chain constant region and the antibody light chain constant region form a dimerization domain for formation of a dimeric antigen receptor (DAR), and
wherein the antibody heavy chain variable region and the antibody light chain variable region form an antigen binding domain that binds CD20; or
(II)
a) a first polypeptide chain comprising a plurality of polypeptide regions ordered from the amino terminus to the carboxyl terminus: (i) an antibody light chain variable region, (ii) an antibody light chain constant region, (iii) a transmembrane region, and (iv) an intracellular region; and
b) a second polypeptide chain comprising a plurality of polypeptide regions ordered from the amino terminus to the carboxyl terminus: (i) an antibody heavy chain variable region and (ii) an antibody heavy chain constant region;
wherein the antibody heavy chain constant region and the antibody light chain constant region form a dimerization domain for formation of a DAR, and wherein the antibody heavy chain variable region and the antibody light chain variable region form an antigen binding domain that binds CD20.

42. (canceled)

43. A nucleic acid molecule encoding a precursor polypeptide comprising a plurality of polypeptide regions ordered from the amino terminus to the carboxyl terminus: (1) a heavy chain leader sequence (2) an antibody heavy chain variable region, (3) an antibody heavy chain constant region, (4) an optional hinge region, (5) a transmembrane region, (6) an intracellular region, (7) a self-cleaving sequence, (8) a light chain leader sequence, (9) an antibody light chain variable region, and (10) an antibody light chain constant region, wherein the self-cleaving sequence permits cleaving the of the precursor polypeptide into a first polypeptide chain and a second polypeptide chain; or
a precursor polypeptide comprising a plurality of polypeptide regions ordered from the amino terminus to the carboxyl terminus: (1) a light chain leader sequence (2) an antibody light chain variable region, (3) an antibody light chain constant region, (4) an optional hinge region, (5) a transmembrane region, (6) an intracellular region, (7) a self-cleaving sequence, (8) a heavy chain leader sequence, (9) an antibody heavy chain variable region, and (10) an antibody heavy chain constant region, wherein the self-cleaving sequence permits cleaving the of the precursor polypeptide into a first and second polypeptide chain.

44. (canceled)

45. The nucleic acid molecule of claim 43, wherein the antibody heavy chain variable region comprises the amino acid sequence of SEQ ID NO:3, or wherein the antibody heavy chain constant region comprises the amino acid sequence of SEO ID NO:4, or
wherein the antibody light chain variable region comprises the amino acid sequence of SEQ ID NO:11, or
wherein the antibody light chain constant region comprises the amino acid sequence of SEQ ID NO:12, or
wherein the hinge region comprises a hinge sequence from an antibody selected from a group consisting of IgG, IgA, IgM, IgE and IgD, or
wherein the hinge region comprises a CD28 hinge region, or
wherein the hinge region comprises a CPPC (SEQ ID NO: 61) or SPPC (SEQ ID NO: 62) amino acid sequence, or
wherein the hinge region comprises the amino acid sequence of SEQ ID NO:5, or
wherein the transmembrane region comprises a transmembrane sequence from CD28, or
wherein the transmembrane region comprises the amino acid sequence of SEQ ID NO:6.

46-55. (canceled)

56. The nucleic acid molecule of claim 43, wherein the intracellular region comprises:
i) a 4-1BB intracellular costimulatory sequence (SEQ ID NO:7); and
ii) a CD3zeta having ITAM 3 (SEQ ID NO:8).

57. (canceled)

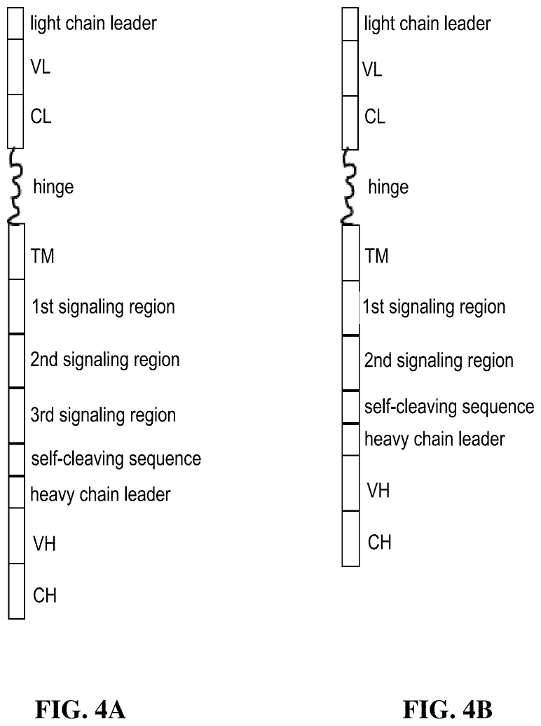

58. The nucleic acid molecule of claim 43, comprising, wherein the precursor polypeptide comprises the amino acid sequence of SEQ ID NO:13,_or wherein the precursor polypeptide comprises the orientation and amino acid sequences shown in FIGS. 4A and 4B.

59. (canceled)

60. A method for treating a subject having a disease, disorder or condition associated with detrimental expression of a tumor antigen in the subject, comprising administering to the subject the population of genetically modified host cells of claim 1, and
optionally wherein the disease is a hematologic cancer selected from the group consisting of non-Hodgkin's lymphoma (NHL) Burkitt's lymphoma (BL), B chronic lymphocytic leukemia (B-CLL), B and T acute lymphocytic leukemia (ALL), T cell lymphoma (TCL), acute myeloid leukemia (AML), hairy cell leukemia (HCL), Hodgkin's Lymphoma (HL), chronic myeloid leukemia (CML) and multiple myeloma (MM).

61. (canceled)


SEQUENCE LISTING (62 sequences)

SEQ ID NO: 1 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 2 - protein, 19 aa, Mus musculus
MEWSWVFLFFLSVTTGVHS

SEQ ID NO: 3 - protein, 121 aa, synthetic construct [sequence omitted]

SEQ ID NO: 4 - protein, 108 aa, synthetic construct
  REGION 1..108: note = Anti-CD20 heavy chain constant region (CH1)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHT

SEQ ID NO: 5 - protein, 42 aa, synthetic construct
  REGION 1..42: note = CD28 hinge
PRKIEVMYPPPYLDNEKSNGTIIHVKGKHLCPSPLFPGPSKP

SEQ ID NO: 6 - protein, 27 aa, synthetic construct
  REGION 1..27: note = CD28 transmembrane domain
FWVLVVVGGVLACYSLLVTVAFIIFWV

SEQ ID NO: 7 - protein, 42 aa, synthetic construct
  REGION 1..42: note = 4-1BB intracellular costimulatory sequence
KRGRKKLLYIFKQPFMRPVQTTQEEDGCSCRFPEEEEGGCEL

SEQ ID NO: 8 - protein, 45 aa, synthetic construct
  REGION 1..45: note = CD3zeta intracellular signaling region, ITAM3 only
RVKFSRSADKGERRRGKGHDGLYQGLSTATKDTYDALHMQALPPR

SEQ ID NO: 9 - protein, 21 aa, Thosea asigna virus
GSGEGRGSLLTCGDVEENPGP

SEQ ID NO: 10 - protein, 20 aa, synthetic construct
  REGION 1..20: note = Light chain leader peptide
MSVPTQVLGLLLLWLTDARC

SEQ ID NO: 11 - protein, 106 aa, synthetic construct [sequence omitted]

SEQ ID NO: 12 - protein, 107 aa, synthetic construct [sequence omitted]

SEQ ID NO: 13 - protein, 658 aa, synthetic construct
  REGION 1..658: note = Anti-CD20 DAR precursor polypeptide
MEWSWVFLFFLSVTTGVHSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTPRKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADKGERRRGKGHDGLYQGLSTATKDTYDALHMQALPPRGSGEGRGSLLTCGDVEENPGPMSVPTQVLGLLLLWLTDARCQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 385 aa, synthetic construct
  REGION 1..385: note = Anti-CD20 DAR 1st polypeptide chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTPRKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADKGERRRGKGHDGLYQGLSTATKDTYDALHMQALPPR

SEQ ID NO: 15 - protein, 213 aa, synthetic construct
  REGION 1..213: note = Anti-CD20 DAR 2nd polypeptide chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - DNA, 1974 bp, synthetic construct
  misc_feature 1..1974: note = sequence encoding the anti-CD20 DAR
  source 1..1974: mol_type = other DNA
atggagtggagctgggtgtttctgttcttcctctccgtcacaaccggcgtgcatagccaagtgcaattgcagcagcccggtgccgaactcgtgaaaccaggagcaagcgtaaagatgtcctgtaaggcatcaggttatacctttaccagctacaacatgcactgggtgaaacaaacgccggggcggggcctcgaatggataggcgcgatatatcccggaaatggcgataccagttacaatcagaagttcaaaggcaaagcgacactgacagctgataagtcttcaagcaccgcctatatgcaactttctagcctgaccagcgaagactccgccgtttattactgtgctcggtccacatactacggaggcgattggtactttaatgtgtggggtgcgggcaccactgtcactgtatcagcggcttccaccaagggcccctccgtgttccctctggcccccagcagcaagagcacatccggaggcaccgccgccctcggatgtctggtgaaggactacttccccgagcctgtcaccgtgtcctggaatagcggcgccctcacctccggcgtgcacaccttccccgctgtcctgcagtcctccggactgtacagcctgtcctccgtcgtgaccgtgcctagctcctccctcggcacccagacctacatctgcaacgtgaaccacaagccttccaacacaaaggtggacaaacgggtggagcccaagtcctgcgacaaaacccacacccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttaaacggggcagaaagaaactcctgtatatattcaaacaaccatttatgagaccagtacaaactactcaagaggaagatggctgtagctgccgatttccagaagaggaggaaggaggatgtgaactgagggtaaaatttagcaggtctgcagataaaggggagaggagacgcgggaagggccatgatggactgtatcagggactttccacagccaccaaggacacctatgacgctctccacatgcaggccctgccccctcgcggaagcggagagggcagaggaagtctgctaacatgcggtgacgtcgaggagaatcctggacctatgtccgtccctacccaggtgctgggcctgctgctgctgtggctgaccgatgctagatgccagatagtcctgagccaatcaccggccatcttgtctgcctctcctggcgaaaaggtgacgatgacttgcagagccagtagctctgtaagctatatacactggttccagcaaaaaccgggctcttctccgaagccgtggatatacgcaacttcaaacctggcgtctggggttcctgtaaggtttagcggcagcggttcaggcacgagctacagccttactatctcccgggttgaggctgaagatgcagccacatactactgtcagcagtggacttcaaatccacctacattcgggggaggcacgaagctggagattaaacgaaccgttgcggcgcctagtgtgttcatattcccgccgtctgatgaacaactcaagtctggaacggcaagtgtggtgtgtctcctgaataatttttatcctagggaagcaaaggtgcagtggaaagtcgataacgcattgcaaagcggtaacagtcaagaatctgtaactgaacaagattctaaagattctacctacagtctctcctccacattgaccctgtcaaaagcagattatgagaagcacaaggtgtacgcatgtgaggtaacacatcaaggactcagcagcccagttacaaaaagtttcaatcgcggggaatgt

SEQ ID NO: 17 - DNA, 1251 bp, synthetic construct
  misc_feature 1..1251: note = sequence encoding the anti-CD20 CAR
  source 1..1251: mol_type = other DNA
atggagtggagctgggtctttctgttctttttaagcgtgaccaccggagtgcactcccagatagtcctgagccaatcaccggccatcttgtctgcctctcctggcgaaaaggtgacgatgacttgcagagccagtagctctgtaagctatatacactggttccagcaaaaaccgggctcttctccgaagccgtggatatacgcaacttcaaacctggcgtctggggttcctgtaaggtttagcggcagcggttcaggcacgagctacagccttactatctcccgggttgaggctgaagatgcagccacatactactgtcagcagtggacttcaaatccacctacattcgggggaggcacgaagctggagattaaaggcggcggcggcagcggtggcggtggctcaggtggtggtggttctcaagtgcaattgcagcagcccggtgccgaactcgtgaaaccaggagcaagcgtaaagatgtcctgtaaggcatcaggttatacctttaccagctacaacatgcactgggtgaaacaaacgccggggcggggcctcgaatggataggcgcgatatatcccggaaatggcgataccagttacaatcagaagttcaaaggcaaagcgacactgacagctgataagtcttcaagcaccgcctatatgcaactttctagcctgaccagcgaagactccgccgtttattactgtgctcggtccacatactacggaggcgattggtactttaatgtgtggggtgcgggcaccactgtcactgtatcagcgcccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttaaacggggcagaaagaaactcctgtatatattcaaacaaccatttatgagaccagtacaaactactcaagaggaagatggctgtagctgccgatttccagaagaagaagaaggaggatgtgaactgagggtaaaatttagcaggtctgcagataaaggggagaggagacgcgggaagggccatgatggactgtatcagggactttccacagccaccaaggacacctatgacgctctccacatgcaggccctgccccctcgc

SEQ ID NO: 18 - protein, 417 aa, synthetic construct
  REGION 1..417: note = Anti-CD20 CAR precursor polypeptide
MEWSWVFLFFLSVTTGVHSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAPRKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADKGERRRGKGHDGLYQGLSTATKDTYDALHMQALPPR

SEQ ID NO: 19 - protein, 15 aa, synthetic construct
  REGION 1..15: note = CAR GS linker
GGGGSGGGGSGGGGS

SEQ ID NO: 20 - DNA, 195 bp, synthetic construct
  misc_feature 1..195: note = Jet promoter
  source 1..195: mol_type = other DNA
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgaca

SEQ ID NO: 21 - DNA, 660 bp, Homo sapiens
  source 1..660: mol_type = genomic DNA
ggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccaca

SEQ ID NO: 22 - DNA, 650 bp, Homo sapiens
  source 1..650: mol_type = genomic DNA
gatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatcttt

SEQ ID NO: 23 - DNA, 19 bp, Homo sapiens
  source 1..19: mol_type = genomic DNA
cagggttctggatatctgt

SEQ ID NO: 24 - DNA, 171 bp, Homo sapiens
  source 1..171: mol_type = genomic DNA
atcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccaca

SEQ ID NO: 25 - DNA, 161 bp, Homo sapiens
  source 1..161: mol_type = genomic DNA
gatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtc

SEQ ID NO: 26 - DNA, 20 bp, synthetic construct
  misc_feature 1..20: note = Forward primer
  misc_feature 1..2: note = phosphorothioate linkage
  misc_feature 3..4: note = phosphorothioate linkage
  modified_base 3: mod_base = cm
  misc_feature 4..5: note = phosphorothioate linkage
  modified_base 4: mod_base = OTHER; note = 2'-O-methyladenosine
  modified_base 5: mod_base = cm
  source 1..20: mol_type = other DNA
atcacgagcagctggtttct

SEQ ID NO: 27 - DNA, 24 bp, synthetic construct
  misc_feature 1..24: note = Reverse primer
  source 1..24: mol_type = other DNA
gacctcatgtctagcacagttttg

SEQ ID NO: 28 - DNA, 645 bp, Homo sapiens
  source 1..645: mol_type = genomic DNA
tgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccct

SEQ ID NO: 29 - DNA, 600 bp, Homo sapiens
  source 1..600: mol_type = genomic DNA
gtaccagctgagagactcactatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttattt

SEQ ID NO: 30 - DNA, 21 bp, Homo sapiens
  source 1..21: mol_type = genomic DNA
gagtctctcagctggtacacg

SEQ ID NO: 31 - DNA, 192 bp, Homo sapiens
  source 1..192: mol_type = genomic DNA
atcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccct

SEQ ID NO: 32 - DNA, 159 bp, Homo sapiens
  source 1..159: mol_type = genomic DNA
gtaccagctgagagactcactatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgc

SEQ ID NO: 33 - DNA, 20 bp, synthetic construct
  misc_feature 1..20: note = Forward primer for generating donor for insertion into Cas12asite
  misc_feature 1: note = 5' phosphate
  source 1..20: mol_type = other DNA
atcacgagcagctggtttct

SEQ ID NO: 34 - DNA, 21 bp, synthetic construct
  misc_feature 1..21: note = Artificial Reverse primer for generating donor for insertion intoCas12a site
  misc_feature 1..2: note = phosphorothioate linkage
  modified_base 1: mod_base = gm
  misc_feature 2..3: note = phosphorothioate linkage
  modified_base 2: mod_base = cm
  misc_feature 3..4: note = phosphorothioate linkage
  modified_base 3: mod_base = OTHER; note = 2'-O-methyladenosine
  source 1..21: mol_type = other DNA
gcactgttgctcttgaagtcc

SEQ ID NO: 35 - protein, 48 aa, Homo sapiens
AKPTTTPAPRPPTPAPTIASQPLSLRPEACRPAAGGAVHTRGLDFAPR

SEQ ID NO: 36 - protein, 88 aa, synthetic construct
  REGION 1..88: note = CD8 hinge + CD28 hinge (long hinge)
AKPTTTPAPRPPTPAPTIASQPLSLRPEACRPAAGGAVHTRGLDFAPRKIEVMYPPPYLDNEKSNGTIIHVKGKHLCPSPLFPGPSKP

SEQ ID NO: 37 - protein, 23 aa, Homo sapiens
IYIWAPLAGTCGVLLLSLVITLY

SEQ ID NO: 38 - protein, 27 aa, Homo sapiens
IISFFLALTSTALLFLLFFLTLRFSVV

SEQ ID NO: 39 - protein, 21 aa, Homo sapiens
LCYLLDGILFIYGVILTALFL

SEQ ID NO: 40 - protein, 41 aa, synthetic construct
  REGION 1..41: note = CD28 co-stimulatory sequence
RSKRSRLLHSDYMNMTPRRPGPTRKHYQPYAPPRDFAAYRS

SEQ ID NO: 41 - protein, 42 aa, Homo sapiens
ALYLLRRDQRLPPDAHKPPGGGSFRTPIQEEQADAHSTLAKI

SEQ ID NO: 42 - protein, 112 aa, synthetic construct
  REGION 1..112: note = CD3zeta, including ITAMs 1, 2, and 3
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 38 aa, synthetic construct
  REGION 1..38: note = CD3zeta ITAM 1
RVKFSRSADAPAYQQGQNQLYNELNLGRREEYDVLDKR

SEQ ID NO: 44 - protein, 47 aa, synthetic construct
  REGION 1..47: note = CD3zeta ITAM 2
RVKFSRSADRGRDPEMGGKPRRKNPQEGLYNELQKDKMAEAYSEIGM

SEQ ID NO: 45 - protein, 45 aa, synthetic construct; identical to SEQ ID NO: 8
  REGION 1..45: note = CD3zeta ITAM 3

SEQ ID NO: 46 - protein, 153 aa, synthetic construct
  REGION 1..153: note = DAR V1 intracellular domain
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 154 aa, synthetic construct
  REGION 1..154: note = DAR V2a intracellular domain
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 153 aa, synthetic construct; identical to SEQ ID NO: 46
  REGION 1..153: note = DAR V2b and CAR (28Z) intracellular domain

SEQ ID NO: 49 - protein, 195 aa, synthetic construct
  REGION 1..195: note = DAR V2c intracellular domain
KRGRKKLLYIFKQPFMRPVQTTQEEDGCSCRFPEEEEGGCELRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 87 aa, synthetic construct
  REGION 1..87: note = DAR V3 and V4 intracellular domain
KRGRKKLLYIFKQPFMRPVQTTQEEDGCSCRFPEEEEGGCELRVKFSRSADKGERRRGKGHDGLYQGLSTATKDTYDALHMQALPPR

SEQ ID NO: 51 - protein, 120 aa, synthetic construct
  REGION 1..120: note = DAR V3 intracellular domain
KRGRKKLLYIFKQPFMRPVQTTQEEDGCSCRFPEEEEGGCELRSKRSRLLHSDYMNMTPRRPGPTRKHYQPYAPPRDFAAYRSRVKFSRSADKGERRRGKGHDGLYQGLSTATKDTYDAL

SEQ ID NO: 52 - protein, 21 aa, Homo sapiens
MALPVTALLLPLALLLHAARP

SEQ ID NO: 53 - protein, 22 aa, synthetic construct
  REGION 1..22: note = P2A self-cleaving sequence
GSGATNFSLLKQAGDVEENPGP

SEQ ID NO: 54 - protein, 23 aa, synthetic construct
  REGION 1..23: note = E2A self-cleaving sequence
GSGQCTNYALLKLAGDVESNPGP

SEQ ID NO: 55 - protein, 25 aa, synthetic construct
  REGION 1..25: note = F2A self-cleaving sequence
GSGVKQTLNFDLLKLAGDVESNPGP

SEQ ID NO: 56 - protein, 9 aa, synthetic construct
  REGION 1..9: note = Hinge sequence
PAPELLGGP

SEQ ID NO: 57 - protein, 12 aa, synthetic construct
  REGION 1..12: note = CH2 region
SVFLFPPKPKDT

SEQ ID NO: 58 - protein, 23 aa, synthetic construct
  REGION 1..23: note = Hinge sequence
EPKSCDKTHTCPPCPAPELLGGP

SEQ ID NO: 59 - protein, 10 aa, synthetic construct
  REGION 1..10: note = Hinge sequence
EPKSCDKTHT

SEQ ID NO: 60 - skipped

SEQ ID NO: 61 - protein, 4 aa, synthetic construct
  REGION 1..4: note = Hinge sequence
CPPC

SEQ ID NO: 62 - protein, 4 aa, synthetic construct
  REGION 1..4: note = Hinge sequence
SPPC